(19) United States
(12) Patent Application Publication
(10) Pub. No.: US 2016/0183537 A1
(43) Pub. Date: Jun. 30, 2016

(54) BACILLUS AMYLOLIQUEFACIENS RTI472 COMPOSITIONS AND METHODS OF USE FOR BENEFITING PLANT GROWTH AND TREATING PLANT DISEASE

(71) Applicant: FMC Corporation, Philadelphia, PA (US)

(72) Inventors: Safiyh Taghavi, Chapel Hill, NC (US); Daniel van der Lelie, Chapel Hill, NC (US); Melanie Ann Rehder Silinski, Hillsborough, NC (US)

(21) Appl. No.: 14/980,842

(22) Filed: Dec. 28, 2015

Related U.S. Application Data
(60) Provisional application No. 62/097,207, filed on Dec. 29, 2014.

(51) Int. Cl. A01N 63/00; A01N 37/10; C12R 1/07; C12N 1/20

(52) U.S. Cl. CPC A01N 63/00; C12N 1/20; A01N 37/10; C12R 1/07

(57) ABSTRACT

Compositions and methods include a new strain of Bacillus amyloliquefaciens having activity against plant pathogens. The compositions are useful for benefiting plant growth and/or conferring protection against a pathogenic infection when applied to plant foliage, flowers, fruits, bark, roots, seeds, callus tissue, grafts, cuttings, surrounding soil or growth medium, and soil or growth medium concomitant with sowing seed and planting callus tissue, grafts, and cuttings. The compositions containing the Bacillus amyloliquefaciens RTI472 strain can be applied alone or in combination with other microbial, biological, or chemical insecticides, fungicides, nematicides, bacteriocides, herbicides, plant extracts, plant growth regulators, or fertilizers. In one example, the Bacillus amyloliquefaciens RTI472 strain can be delivered to the plant as part of an integrated pest management program, with other microbial or chemical insecticides, fungicides, nematicides, bacteriocides, herbicides, plant extracts, and plant growth regulators.

### CROSS REFERENCE TO RELATED APPLICATIONS

This application claims the benefit of U.S. provisional application No. 62/097,207 filed Dec. 29, 2014, the disclosure of which is hereby incorporated herein by reference in its entirety.

### TECHNICAL FIELD

The presently disclosed subject matter relates to compositions comprising an isolated strain of Bacillus amyloliquefaciens RTI472 for application to plant foliage, plant fruits and flowers, plant seeds and roots, and the soil surrounding plants to benefit plant growth and to treat plant plant disease(s).

### BACKGROUND

A number of microorganisms having beneficial effects on plant growth and health are known to be present in the soil, to live in association with plants specifically in the root zone (Plant Growth Promoting Rhizobacteria “PGPR”), or to reside as endophytes within the plant. Their beneficial plant growth promoting properties include nitrogen fixation, iron chelation, phosphate solubilization, inhibition of non-beneficial microrganisms, resistance to pests, Induced Systemic Resistance (ISR), Systemic Acquired Resistance (SAR), decomposition of plant material in soil to increase useful soil organic matter, and synthesis of phytohormones such as indole-acetic acid (IAA), acetoin and 2,3-butanediol that stimulate plant growth, development and responses to environmental stresses such as drought. In addition, these microorganisms can interfere with a plant's ethylene stress response by breaking down the precursor molecule, 1-aminocyclopropane-1-carboxylate (ACC), thereby stimulating plant growth and slowing fruit ripening. These beneficial microorganisms can improve soil quality, plant growth, yield, and quality of crops. Various microorganisms exhibit biological activity such as to be useful to control plant diseases. Such biopesticides (living organisms and the compounds naturally produced by these organisms) are safer and more biodegradable than synthetic fertilizers and pesticides.

Fungal phytopathogens, including but not limited to Botrytis spp. (e.g. Botrytis cinerea), Fusarium spp. (e.g. F. oxysporum and F. graminearum), Rhizoctonia spp. (e.g. R. solani), Magnaporthe spp., Mycosphaerella spp., Puccinia spp. (e.g. P. recondita), Phytopthora spp. and Phakopsora spp. (e.g. P. pachyrhizi), are one type of plant pest that can cause servere economic losses in the agricultural and horticultural industries. Chemical agents can be used to control fungal phytopathogens, but the use of chemical agents suffers from disadvantages including high cost, lack of efficacy, emergence of resistant strains of the fungi, and undesirable environmental impacts. In addition, such chemical treatments tend to be indiscriminant and may adversely affect beneficial bacteria, fungi, and arthropods in addition to the plant pathogen at which the treatments are targeted. A second type of plant pest are bacterial pathogens, including but not limited to Erwinia spp. (such as Erwinia chrysanthemi), Pantoea spp. (such as P. citrea), Xanthomonas (e.g. Xanthomonas campestris), Pseudomonas spp. (such as P. syringae) and Ralstonia spp. (such as R. soleacearum) that cause servere economic losses in the agricultural and horticultural industries. Similar to pathogenic fungi, the use of chemical agents to treat these bacterial pathogens suffers from disadvantages. Viruses and virus-like organisms comprise a third type of plant disease-causing agent that is hard to control, but to which bacterial microorganisms can provide resistance in plants via induced systemic resistance (ISR). Thus, microorganisms that can be applied as biofertilizer and/or biopesticide to control pathogenic fungi, viruses, and bacteria are desirable and in high demand to improve agricultural sustainability. A final type of plant pathogen includes plant pathogenic nematodes and insects, which can cause severe damage and loss of plants.

Some members of the species Bacillus have been reported as biocontrol strains, and some have been applied in commercial products (Joseph W. Kloepper, et al. 2004, Phytopathology Vol. 94, No. 11, 1259-1266). For example, strains currently being used in commercial biocontrol products include: Bacillus pumilus strain QST2808, used as active ingredient in SONATA and BALLAD-PLUS, produced by BAYER CROP SCIENCE; Bacillus pumilus strain GB34, used as active ingredient in YIELDSHIELD, produced by BAYER CROP SCIENCE; Bacillus subtilis strain QST713, used as the active ingredient of SERENADE, produced by BAYER CROP SCIENCE; Bacillus subtilis strain GBO3, used as the active ingredient in KODIAK and SYSTEM3, produced by HELENA CHEMICAL COMPANY. Various strains of Bacillus thuringiensis and Bacillus firmus have been applied as biocontrol agents against nematodes and vector insects and these strains serve as the basis of numerous commercially available biocontrol products, including NORTICA and PONCHO-VOTIVO, produced by BAYER CROP SCIENCE. In addition, Bacillus strains currently being used in commercial biostimulant products include: Bacillus amyloliquefaciens strain FZB42 used as the active ingredient in RHIZOVITAL 42, produced by ABiTEP GmbH, as well as various other Bacillus subtilus species that are included as whole cells including their fermentation extract in biostimulant products, such as FULZYME produced by JHBiotech Inc.

The presently disclosed subject matter provides microbial compositions and methods for their use in benefiting plant growth and disease prevention and control.

### SUMMARY OF THE INVENTION

In one embodiment, a composition is provided comprising a biologically pure culture of Bacillus amyloliquefaciens RTI472 deposited as ATCC No. PTA-121166, or a mutant thereof having all the identifying characteristics thereof, for application to a plant for one or both of benefiting plant growth or conferring protection against a pathogenic infection in a susceptible plant.

In one embodiment, a plant seed is provided coated with a composition comprising spores of a biologically pure culture of Bacillus amyloliquefaciens RTI472 deposited as ATCC No. PTA-121166, or a mutant thereof having all the identifying characteristics thereof, present in an amount suitable to benefit plant growth and/or to confer protection against a pathogenic infection in a susceptible plant.

In one embodiment, a composition is provided for one or both of benefiting plant growth or conferring protection against pathogenic infection in a susceptible plant, the composition comprising a biologically pure culture of Bacillus amyloliquefaciens RTI472 deposited as ATCC No. PTA-121166, or a mutant thereof having all the identifying characteristics thereof, in an amount suitable to benefit plant growth and/or to confer protection against pathogenic infection in the susceptible plant; and one or a combination of a microbial, a biological, or a chemical insecticide, fungicide, nematicide, bacteriocide, herbicide, plant extract, plant growth regulator, or fertilizer, in an amount suitable to benefit plant growth and/or to confer protection against pathogenic infection in the susceptible plant.

In one embodiment, a method is provided for one or both of benefiting growth of a plant or conferring protection against pathogenic infection in a susceptible plant, the method comprising delivering a composition comprising a biologically pure culture of Bacillus amyloliquefaciens RTI472 deposited as ATCC No. PTA-121166, or a mutant thereof having all the identifying characteristics thereof to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit the plant growth and/or to confer protection against pathogenic infection in the susceptible plant.

In one embodiment, a method is provided for one or both of benefiting growth of a plant or conferring protection against pathogenic infection in a susceptible plant, the method comprising delivering a composition comprising a biologically pure culture of Bacillus amyloliquefaciens RTI472 deposited as ATCC No. PTA-121166, or a mutant thereof having all the identifying characteristics thereof, in an amount suitable for benefiting the plant growth and/or conferring protection against the pathogenic infection; and one or a combination of a microbial, a biological, or a chemical insecticide, fungicide, nematicide, bacteriocide, herbicide, plant extract, plant growth regulator, or fertilizer in an amount suitable for benefiting the plant growth and/or conferring protection against the pathogenic infection, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for one or both of benefiting growth of a plant or conferring protection against pathogenic infection in a susceptible plant, the method comprising delivering a combination of a first composition comprising a biologically pure culture of Bacillus amyloliquefaciens RTI472 deposited as ATCC No. PTA-121166, or a mutant thereof having all the identifying characteristics thereof in an amount suitable to benefit the plant growth and/or to confer protection against pathogenic infection in the susceptible plant; and a second composition comprising one or a combination of a microbial, a biological, or a chemical insecticide, fungicide, nematicide, bacteriocide, herbicide, plant extract, plant growth regulator, or fertilizer, in an amount suitable to benefit the plant growth and/or to confer protection against pathogenic infection in the susceptible plant, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for one or both of benefiting growth of a plant or conferring protection against pathogenic infection in a susceptible plant, the method comprising: planting a seed of the plant or regenerating a vegetative cutting/tissue of the plant in a suitable growth medium, wherein the seed has been coated or the vegetative cutting/tissue has been inoculated with a composition comprising a biologically pure culture of Bacillus amyloliquefaciens RTI472 deposited as ATCC PTA-121166, or a mutant thereof having all the identifying characteristics thereof, wherein growth of the plant from the seed or the vegetative cutting/tissue is benefited and/or protection against pathogenic infection is conferred.

In one embodiment, a method is provided for benefiting plant growth by conferring protection against or reducing pathogenic infection in a susceptible plant while minimizing the build-up of resistance against the treatment, the method comprising delivering to the susceptible plant in separate applications and in altering time intervals a first composition and a second composition, wherein each of the first and second compositions are delivered in an amount suitable to to confer protection against or reduce pathogenic infection in the plant, wherein the first composition comprises a biologically pure culture of Bacillus amyloliquefaciens RTI472 deposited as ATCC No. PTA-121166, or a mutant thereof having all the identifying characteristics thereof, and wherein the second composition comprises one or more chemical active agents having fungicidal or a bacteriocidal properties, and wherein the first and second compositions are delivered in the altering time intervals to one or a combination of foliage of the plant, bark of the plant, fruit of the plant, flowers of the plant, seed of the plant, roots of the plant, a cutting of the plant, a graft of the plant, callus tissue of the plant, or soil or growth medium surrounding the plant, wherein the total amount of the chemical active agent(s) required to confer protection against and/or reduce the pathogenic infection is decreased and the build-up of resistance against the treatment is minimized.

In one embodiment of the present invention, a composition is provided, the composition including at least one of an isolated Fengycin MA compound, an isolated Fengycin MB compound, an isolated Fengycin MC compound, an isolated Dehydroxyfengycin MA compound, an isolated Dehydroxyfengycin MB compound, an isolated Fengycin H compound, an isolated Fengycin I compound, and an isolated Dehyroxyfengycin I compound in an amount suitable to confer one or both of a growth benefit on the plant or protection against a pathogenic infection in a susceptible plant, the Fengycin and Dehyroxyfengycin compounds having the formula:

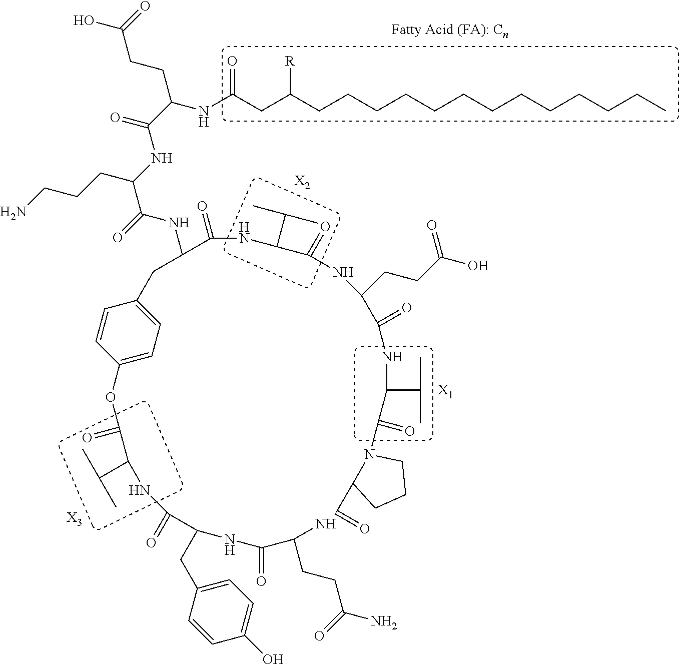

wherein R is OH, n ranges from 8 to 20, FA is linear, iso, or anteiso and: X₁ is Ala, X₂ is Thr, and X₃ is Met for Fengycin MA; X₁ is Val, X₂ is Thr, and X₃ is Met for Fengycin MB; X₁ is Aba, X₂ is Thr, and X₃ is Met for Fengycin MC; X₁ is Val, X₂ is Thr, and X₃ is Hcy for Fengycin H; and X₁ is Ile, X₂ is Thr, and X₃ is Ile for Fengycin I; or wherein R is H, n ranges from 8 to 20, FA is linear, iso, or anteiso and: X₁ is Ala, X₂ is Thr, and X₃ is Met for Dehydroxyfengycin MA; X₁ is Val, X₂ is Thr, and X₃ is Met for Dehydroxyfengycin MB; and X₁ is Ile, X₂ is Thr, and X₃ is Ile for Dehydroxyfengycin I.

In one embodiment, a composition is provided comprising one or both of an isolated Ericin S compound having molecular weight equal to 3341.6 Da and an isolated Ericin A compound having molecular weight equal to 2985.4 Da in an amount suitable to confer protection against a pathogenic infection in a susceptible plant.

In one embodiment, an extract is provided of a biologically pure culture of Bacillus amyloliquefaciens RTI472 deposited as ATCC No. PTA-121166, the extract including a Fengycin-MA, -MB, -MC, -H, and -I compound and a Dehydroxyfengycin-MA, -MB, and -I compound and one or a combination of additional Fengycin- and Dehydroxyfengycin-like compounds listed in Table XVII.

In one embodiment, an extract is provided of a biologically pure culture of Bacillus amyloliquefaciens, the extract including at least one of an isolated Ericin S compound having molecular weight equal to 3341.6 Da or an isolated Ericin A compound having molecular weight equal to 2985.4 Da.

In one embodiment, a composition is provided for benefiting plant growth, the composition comprising: a biologically pure culture of Bacillus amyloliquefaciens RTI472 deposited as ATCC No. PTA-121166, or a mutant thereof having all the identifying characteristics thereof; and a bifenthrin insecticide.

In one embodiment, a composition is provided for benefiting plant growth, the composition comprising: a biologically pure culture of Bacillus amyloliquefaciens RTI472 deposited as ATCC No. PTA-121166, or a mutant thereof having all the identifying characteristics thereof; and a fungicide comprising one or a combination of an extract from Lupinus albus, a BLAD polypeptide, or a fragment of a BLAD polypeptide.

In one embodiment, a product is provided comprising: a first component comprising a biologically pure culture of Bacillus amyloliquefaciens RTI472 deposited as ATCC No. PTA-121166, or a mutant thereof having all the identifying characteristics thereof; a second component comprising one or a combination of a microbial, a biological, or a chemical insecticide, fungicide, nematicide, bacteriocide, herbicide, plant extract, plant growth regulator, or fertilizer, wherein the first and second components are separately packaged, and wherein each component is in an amount suitable for one or both of benefiting plant growth or conferring protection against a pathogenic infection in a susceptible plant; and optionally instructions for delivering in an amount suitable to benefit plant growth, a combination of the first and second compositions to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a composition is provided comprising: a biologically pure culture of one or more microorganisms having properties beneficial to one or both of plant growth or plant health; and a yeast extract, for application to a plant for one or both of benefiting plant growth or conferring protection against a pathogenic infection in a susceptible plant.

### BRIEF DESCRIPTION OF THE FIGURES

Having thus described the presently disclosed subject matter in general terms, reference will now be made to the accompanying Figures described below.

FIG. 1 shows a schematic diagram of the genomic organization surrounding and including a lantibiotic antibiotic biosynthesis operon found in Bacillus amyloliquefaciens strain RTI472 (top) as compared to the corresponding regions for two Bacillus amyloliquefaciens reference strains, Bacillus amyloliquefaciens FZB42 (middle) and Bacillus amyloliquefaciens TrigoCor1448 (bottom), with the percent sequence identity to RTI472 shown below each arrow, according to one or more embodiments of the present disclosure. Further analysis revealed the lantipeptide synthesis operon to produce an Ericin S molecule of MW=3341.6 Da and an Ericin A molecule of MW of 2985.4 Da, which is unique for a Bacillus amyloliquefaciens strain.

FIG. 2A is a graph of metabolite profiles for the Bacillus amyloliquefaciens RTI472 strain and an unpublished Bacillus amyloliquefaciens strain designated “FB005” showing that the RTI472 strain produces an Ericin S molecule (MW=3341.6 Da) and an Ericin A molecule (MW=2985.4 Da), while no Ericin A or Ericin S was detected for the FB005 strain. FIG. 2B is a table showing a comparison between a lantipeptide biosynthesis cluster identified in the RTI472 strain and 7 reference Bacillus amyloliquefaciens genomes, which shows the loss of synteny in the region for the 7 reference Bacillus amyloliquefaciens genomes. This lantipeptide biosynthesis cluster was identified as an Ericin S biosynthetic cluster in the RTI472 strain, while none of the 7 reference strains harbor the functional Ericin S biosynthetic cluster. FIG. 2C is a table showing a comparison between a lantipeptide biosynthesis cluster identified in the RTI472 strain and 7 reference Bacillus amyloliquefaciens genomes, which shows the loss of synteny in the region for the 7 reference Bacillus amyloliquefaciens genomes. This lantipeptide biosynthesis cluster was identified as an Ericin S biosynthetic cluster in the RTI472 strain, while none of the 7 reference strains harbor the functional Ericin S biosynthetic cluster. These figures show a comparison between metabolite profiles and a lantipeptide biosynthesis cluster identified as an Ericin S biosynthetic cluster in the RTI472 strain that is absent in other reference Bacillus amyloliquefaciens strains according to one or more embodiments of the present invention.

FIG. 3 is a bar graph showing the % disease control (mean) on the y axis 10 days after infection with bean rust (Uromyces appendiculatus) following treatment with each of: RTI472 spores in Spent Fermentation Broth (SFB) (applied at 1×10⁸ cfu/ml), TACTIC (applied at 0.1875% to all formulations and also used as a blank control), Tebuconazole (applied at 50 g a.i./ha), Chlorothalonil (applied at 500 g a.i./ha), SERENADE OPTIMUM (applied at 1×10⁸ cfu/ml), and SERENADE OPTIMUM (applied at 4×10⁸ cfu/ml) according to one or more embodiments of the present invention. The non-treated controls resulted in 23% disease. Values followed by the same letter are not significantly different (p=0.10).

FIG. 4 is a bar graph showing the % disease control (mean) on the y axis in soybean plants 9 days after being placed in proximity with soybean plants infected with Microsphaera diffusa following treatment with each of: RTI472 spores in Spent Fermentation Broth (SFB) (applied at 1×10⁸ cfu/ml), SERENADE OPTIMUM (applied at 1×10⁸ cfu/ml), and HORIZON (Tebuconazole applied at a 50 g a.i./ha) according to one or more embodiments of the present invention. In addition, each of the treatments included TACTIC (applied at 0.1875%) as an adjuvant. The non-treated control resulted in 70% disease severity. Values followed by the same letter are not significantly different (p=0.10).

FIG. 5 is a bar graph showing the % disease control (mean) on the y axis 7 days after infection with Pepper Botrytis Blight (Botrytis cinerea), following treatment with each of: RTI472 spores in Spent Fermentation Broth (SFB) (applied at 1×10⁸ cfu/ml), TACTIC (applied at 0.1875% to all formulations and used as a blank control), Tebuconazole (applied at 50 g a.i./ha), Chlorothalonil (applied at 500 g a.i./ha), SERENADE OPTIMUM (applied at 1×10⁸ cfu/ml), and SERENADE OPTIMUM (applied at 4×10⁸ cfu/ml). The non-treated controls resulted in 6% disease according to one or more embodiments of the present invention. Values followed by the same letter are not significantly different (p=0.10).

FIG. 6 is a bar graph showing the % disease control (mean) on the y axis 3 days after infection with Pepper Botrytis Blight (Botrytis cinerea) following treatment with each of: RTI472 spores in Spent Fermentation Broth (SFB) (applied at 2.5×10⁶, 1×10⁷, 2.5×10⁷, 1×10⁸, and 2.5×10⁸ cfu/ml) and SERENADE OPTIMUM (applied at 2.5×10⁶, 1×10⁷, 2.5×10⁷, 1×10⁸, and 2.5×10⁸ cfu/ml) as compared to TACTIC (applied at 0.1875% to all formulations and also used as a blank control), SERENADE OPTIMUM (applied at 4×10⁸), and Tebuconazole (applied at 50 g a.i./ha) according to one or more embodiments of the present invention. The non-treated controls resulted in 30% disease. Values followed by the same letter are not significantly different (p=0.10).

FIG. 7 is a bar graph showing the % disease control (mean) on the y axis 4 days after infection with increasing amounts of Pepper Botrytis Blight (Botrytis cinerea) following treatment with each of: RTI472 spores in Spent Fermentation Broth (SFB) (applied at 1.0×10⁸ cfu/ml) and SERENADE OPTIMUM (applied at 1×10⁸ and 4×10⁸ cfu/ml) as compared to TACTIC (applied at 0.1875% to all formulations and also used as a blank control) and Tebuconazole (HORIZON; applied at 50 g a.i./ha) according to one or more embodiments of the present invention. The percent disease in the non-treated controls as a function of infection with increasing amounts of Botrytis cinerea (conidia/ml) was 50 k=15%, 100 k=30%, 500 k=65%, 1M=65%, and 2M=65%, respectively. Values followed by the same letter are not significantly different (p=0.10).

FIG. 8A is an image of pepper plants 4 days after infection with 50 k conidia/ml of Pepper Botrytis Blight (Botrytis cinerea) following treatment with SERENADE OPTIMUM (applied at 1×10⁸ cfu/ml) according to one or more embodiments of the present invention. FIG. 8B is an image of pepper plants 4 days after infection with 50 k conidia/ml of Pepper Botrytis Blight (Botrytis cinerea) following treatment with RTI472 spores in Spent Fermentation Broth (SFB) (applied at 1×10⁸ cfu/ml) according to one or more embodiments of the present invention. FIG. 8C is an image of pepper plants 4 days after infection with 50 k conidia/ml of Pepper Botrytis Blight (Botrytis cinerea) following treatment with TACTIC (applied at 0.1875% to all formulations and also used as a blank control) according to one or more embodiments of the present invention. FIG. 8D is an image of pepper plants 4 days after infection with 50 k conidia/ml of Pepper Botrytis Blight (Botrytis cinerea) following treatment with Infected Control according to one or more embodiments of the present invention.

FIG. 9A is an image of pepper plants 4 days after infection with 100 k conidia/ml of Pepper Botrytis Blight (Botrytis cinerea) following treatment with SERENADE OPTIMUM (applied at 1×10⁸ cfu/ml) according to one or more embodiments of the present invention. FIG. 9B is an image of pepper plants 4 days after infection with 100 k conidia/ml of Pepper Botrytis Blight (Botrytis cinerea) following treatment with RTI472 spores in Spent Fermentation Broth (SFB) (applied at 1×10⁸ cfu/ml) according to one or more embodiments of the present invention. FIG. 9C is an image of pepper plants 4 days after infection with 100 k conidia/ml of Pepper Botrytis Blight (Botrytis cinerea) following treatment with TACTIC (applied at 0.1875% to all formulations and also used as a blank control) according to one or more embodiments of the present invention. FIG. 9D is an image of pepper plants 4 days after infection with 100 k conidia/ml of Pepper Botrytis Blight (Botrytis cinerea) following treatment withlnfected Control according to one or more embodiments of the present invention.

FIG. 10A is an image of pepper plants 4 days after infection with 500 k conidia/ml of Pepper Botrytis Blight (Botrytis cinerea) following treatment with SERENADE OPTIMUM (applied at 1×10⁸ cfu/ml) according to one or more embodiments of the present invention. FIG. 10B is an image of pepper plants 4 days after infection with 500 k conidia/ml of Pepper Botrytis Blight (Botrytis cinerea) following treatment with RTI472 spores in Spent Fermentation Broth (SFB) (applied at 1×10⁸ cfu/ml) according to one or more embodiments of the present invention. FIG. 10C is an image of pepper plants 4 days after infection with 500 k conidia/ml of Pepper Botrytis Blight (Botrytis cinerea) following treatment with TACTIC (applied at 0.1875% to all formulations and used as a blank control) according to one or more embodiments of the present invention. FIG. 10D is an image of pepper plants 4 days after infection with 500 k conidia/ml of Pepper Botrytis Blight (Botrytis cinerea) following treatment with Infected Control according to one or more embodiments of the present invention.

FIG. 11A is an image of pepper plants 4 days after infection with 1M conidia/ml of Pepper Botrytis Blight (Botrytis cinerea) following treatment with SERENADE OPTIMUM (applied at 1×10⁸ cfu/ml) according to one or more embodiments of the present invention. FIG. 11B is an image of pepper plants 4 days after infection with 1M conidia/ml of Pepper Botrytis Blight (Botrytis cinerea) following treatment with RTI472 spores in Spent Fermentation Broth (SFB) (applied at 1×10⁸ cfu/ml) according to one or more embodiments of the present invention. FIG. 11C is an image of pepper plants 4 days after infection with 1M conidia/ml of Pepper Botrytis Blight (Botrytis cinerea) following treatment with TACTIC (applied at 0.1875% to all formulations and used as a blank control) according to one or more embodiments of the present invention. FIG. 11D is an image of pepper plants 4 days after infection with 1M conidia/ml of Pepper Botrytis Blight (Botrytis cinerea) following treatment with Infected Control according to one or more embodiments of the present invention.

FIG. 12A is an image of pepper plants 4 days after infection with 2M conidia/ml of Pepper Botrytis Blight (Botrytis cinerea) following treatment with SERENADE OPTIMUM (applied at 1×10⁸ cfu/ml) according to one or more embodiments of the present invention. FIG. 12B is an image of pepper plants 4 days after infection with 2M conidia/ml of Pepper Botrytis Blight (Botrytis cinerea) following treatment with RTI472 spores in Spent Fermentation Broth (SFB) (applied at 1×10⁸ cfu/ml) according to one or more embodiments of the present invention. FIG. 12C is an image of pepper plants 4 days after infection with 2M conidia/ml of Pepper Botrytis Blight (Botrytis cinerea) following treatment with TACTIC (applied at 0.1875% to all formulations and also used as a blank control) according to one or more embodiments of the present invention. FIG. 12D is an image of pepper plants 4 days after infection with 2M conidia/ml of Pepper Botrytis Blight (Botrytis cinerea) following treatment with Infected Control according to one or more embodiments of the present invention.

FIG. 13 shows graphs of the percent of fruits infected with Botrytis cinerea pathogen in the untreated control (“UT”) in each of four independent strawberry field trials to determine antagonism of the RTI472 strain against this pathogen according to one or more embodiments of the present invention.

FIG. 14 is a schematic diagram showing both previously reported Fengycin-type and Dehydroxyfengycin-type cyclic lipopeptides produced by microbial species including Bacillus amyloliquefaciens and newly identified (shown in bold type) Fengycin- and Dehydroxyfengycin-type molecules produced by the Bacillus amyloliquefaciens RTI472 isolate according to one or more embodiments of the present invention.

FIG. 15 is a graph showing the percentage of recovered lipopeptides from RTI472 spent fermentation broth (SFB) after acid precipitation according to one or more embodiments of the present invention. The terms “472-AP-Pellet” and “472-AP-Supernatant” refer to the resuspended pellet and supernatant, respectively, obtained after acid precipitation of the centrifuged SFB. The percentage was calculated and compared based on the integrated ion abundance of each lipopeptide from the RTI472 spent fermentation broth (472-SFB).

FIG. 16A shows Botrytis cinerea spotted on 869 agar plates with RTI472 spent fermentation broth (472-SFB). FIG. 16B shows Botrytis cinerea spotted on 869 agar plates with resuspended pellet material obtained after acid precipitation plus centrifugation (472-AP-Pellet). FIG. 16C shows Fusarium graminearum spotted on 869 agar plates with RTI472 spent fermentation broth (472-SFB). FIG. 16D shows Fusarium graminearum spotted on 869 agar plates with resuspended pellet material obtained after acid precipitation plus centrifugation (472-AP-Pellet). FIGS. 16A-16D are images of a plate assay showing control of Botrytis cinerea and Fusarium graminearum by isolated metabolites in acid precipitates of spent fermentation broth (SFB) of B. amyloliquefaciens RTI472 according to one or more embodiments of the present invention. For positive controls, 10 μl of cell culture of RTI472 was spotted on the left side of each plate next to the fungus.

### DETAILED DESCRIPTION

The terms “a,” “an,” and “the” refer to “one or more” when used in this application, including the claims. Thus, for example, reference to “a plant” includes a plurality of plants, unless the context clearly is to the contrary.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

For the purposes of this specification and claims, the terms “metabolite” and “compound” are used interchangeably when used in connection with compounds having antimicrobial activity that are produced by the RTI472 strain or another Bacillus amyloliquefaciens strain.

As used herein, the phrase “a biologically pure culture of a bacterial strain” refers to one or a combination of: spores of the biologically pure fermentation culture of a bacterial strain, vegetative cells of the biologically pure fermentation culture of a bacterial strain, one or more products of the biologically pure fermentation culture of a bacterial strain, a culture solid of the biologically pure fermentation culture of a bacterial strain, a culture supernatant of the biologically pure fermentation culture of a bacterial strain, an extract of the biologically pure fermentation culture of the bacterial strain, and one or more metabolites of the biologically pure fermentation culture of a bacterial strain.

In certain embodiments of the present invention, compositions and methods are provided that include a biologically pure culture of a newly identified strain of Bacillus amyloliquefaciens for application to a plant for one or both of benefiting plant growth or conferring protection against a pathogenic infection in a susceptible plant. In the compositions and methods of the present invention, the growth benefit of the plant is exhibited by improved seedling vigor, improved root development, improved plant growth, improved plant health, increased yield, improved appearance, improved resistance to plant pathogens, reduced pathogenic infection, or a combination thereof.

A plant-associated bacterium, identified as belonging to the species Bacillus amyloliquefaciens, was isolated from the root of American ginseng grown in North Carolina and subsequently tested for plant pathogen antagonistic properties. More specifically, the isolated bacterial strain was identified as a new strain of Bacillus amyloliquefaciens through sequence analysis of highly conserved 16S rRNA and rpoB genes (see EXAMPLE 1). The 16S RNA sequence of the new bacterial isolate (designated “Bacillus amyloliquefaciens RTI472”) was determined to be identical to the 16S rRNA gene sequence of three other known strains of Bacillus amyloliquefaciens, Bacillus amyloliquefaciens strain NS6 (KF177175), Bacillus amyloliquefaciens strain FZB42 (NR_075005), and Bacillus subtilis subsp. subtilis strain DSM 10 (NR_027552). In addition, it was determined that the rpoB sequence of RTI472 has the highest level of sequence similarity to the known Bacillus amyloliquefaciens AS43.3 strain (i.e., >99% sequence identity); however, there is a 10 nucleotide difference on the DNA level, indicating that RTI472 is a new strain of Bacillus amyloliquefaciens RTI472. The strain of Bacillus amyloliquefaciens RTI472 was deposited on 17 Apr. 2014 under the terms of the Budapest Treaty on the International Recognition of the Deposit of Microorganisms for the Purposes of Patent Procedure at the American Type Culture Collection (ATCC) in Manassas, Va., USA and bears the Patent Accession No. PTA-121166.

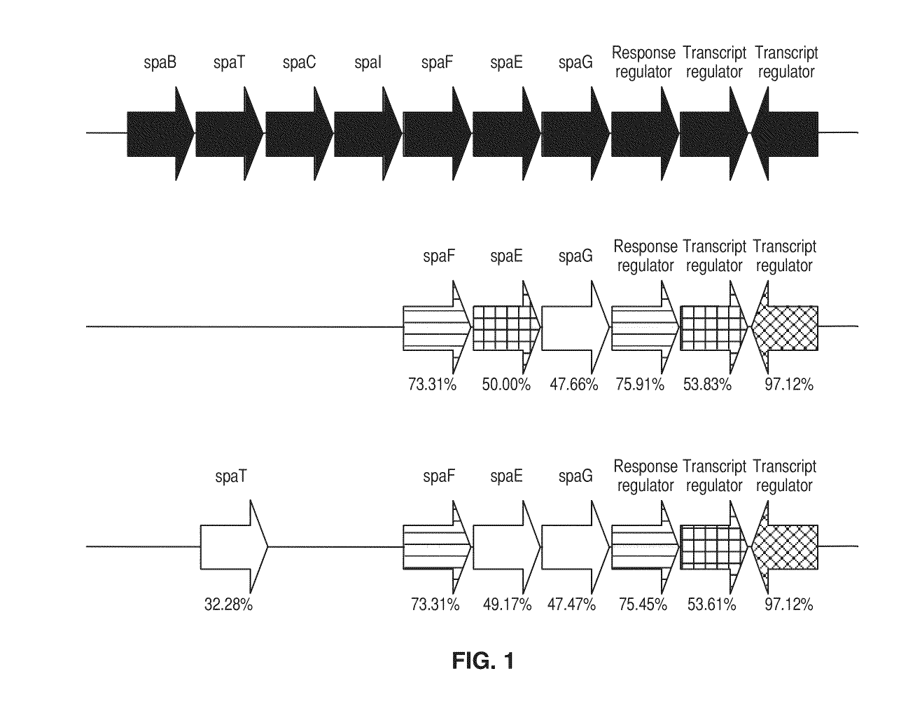

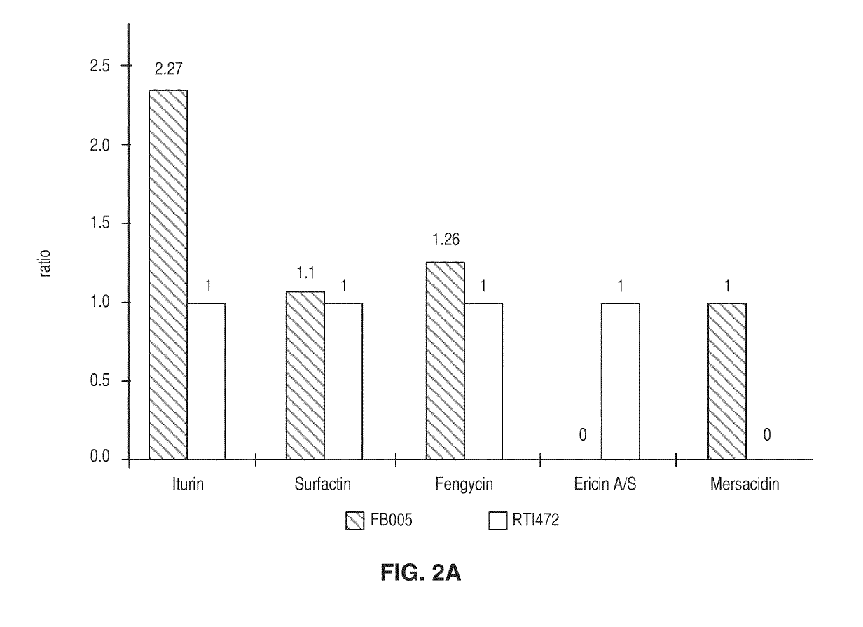

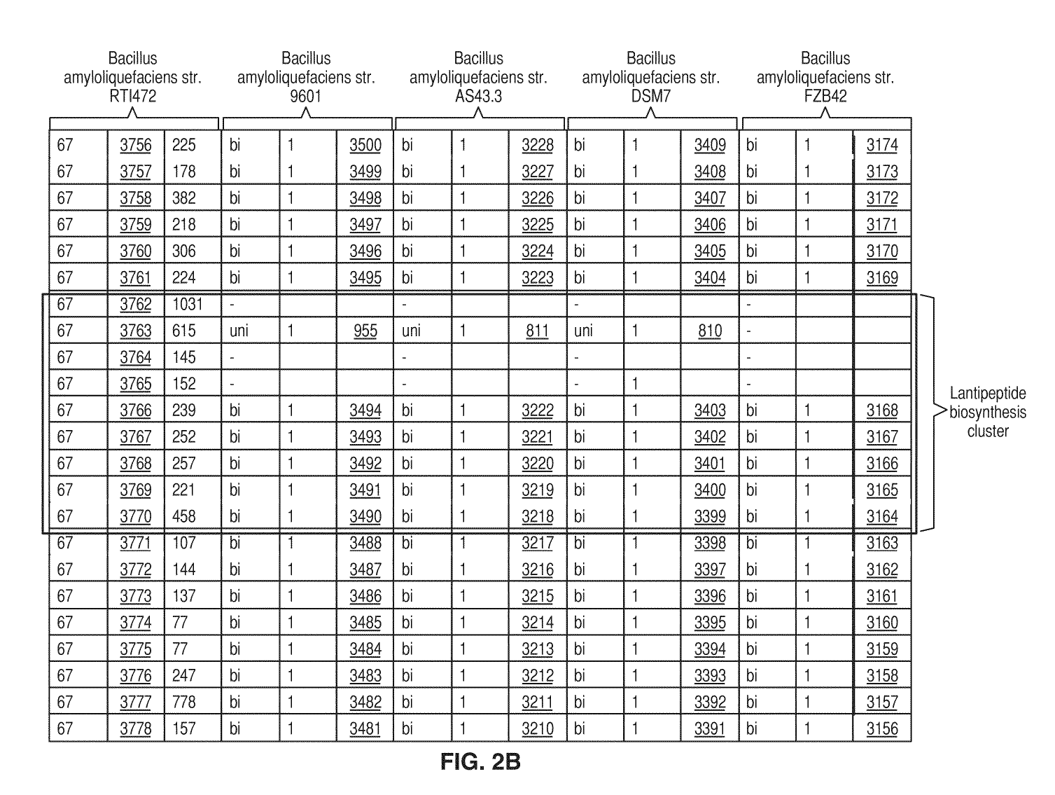

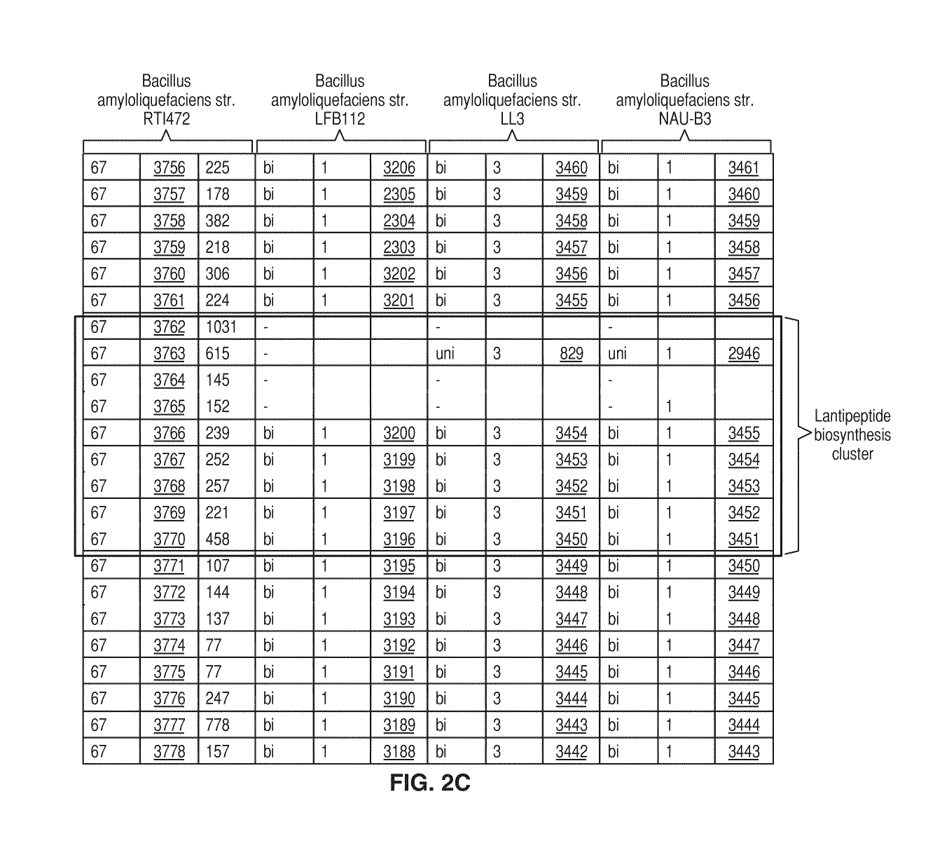

Further sequence analysis of the genome of the Bacillus amyloliquefaciens RTI472 strain revealed that the strain has genes related to lantibiotic biosynthesis for which homologues are lacking in the other closely related Bacillus amyloliquefaciens RTI472 strains (see EXAMPLE 2). This is illustrated in FIG. 1 which shows a schematic diagram of the genomic organization of the lantibiotic biosynthetic cluster found in Bacillus amyloliquefaciens RTI472 and the corresponding region for two known Bacillus amyloliquefaciens reference strains, FZB42 (middle) and TrigoCor1448 (bottom), shown below the RTI472 strain. It can be observed from FIG. 1 that FZB42 and TrigoCor1448 strains lack many of the genes present in this cluster, and there is a low degree of sequence identity within a number of the genes that are present. This lantipeptide biosynthesis cluster in the RTI472 strain was identified as an Ericin S biosynthetic cluster. The metabolite profile of the RTI472 strain was analyzed using HPLC/MS/MS which showed that the newly identified RTI472 strain produces an Ericin S molecule of MW=3341.6 Da and a Ericin A molecule of MW of 2985.4 Da. FIG. 2A shows a graph of the metabolite profile for the Bacillus amyloliquefaciens RTI472 strain and an unpublished Bacillus amyloliquefaciens strain designated “FB005”. The profile shows that the RTI472 strain produces the Ericin A and Ericin S peptides, while no Ericin A or Ericin S was detected for the FB005 strain. For the purposes of the specification and claims, the terms “Ericin S”, “Ericin S molecule”, “Ericin S compound”, and “Ericin S peptide” are herein used interchangeably. Similarly, for the purposes of the specification and claims, the terms “Ericin A”, “Ericin A molecule”, “Ericin A compound”, and “Ericin A peptide” are herein used interchangeably.

In addition, further comparative genomics with a number of additional Bacillus amyloliquefaciens reference strains similarly showed that these strains also lack the lantibiotic biosynthesis cluster. FIGS. 2B-2C are tables showing a comparison between a lantipeptide biosynthesis cluster identified in the RTI472 strain and 7 reference Bacillus amyloliquefaciens genomes, which shows the loss of synteny in the region for the 7 reference Bacillus amyloliquefaciens genomes. The data indicate that the newly identified RTI472 has a unique lantibiotic biosynthesis pathway. This lantipeptide biosynthesis cluster was identified as an Ericin S biosynthetic cluster in the RTI472 strain and none of the 7 reference strains harbor the functional Ericin S biosynthetic cluster. Thus, the newly identified RTI472 strain possesses a lantipeptide synthesis pathway that produces an Ericin S molecule of MW=3341.6 Da and a Ericin A molecule of MW of 2985.4 Da, which is unique for a Bacillus amyloliquefaciens strain.

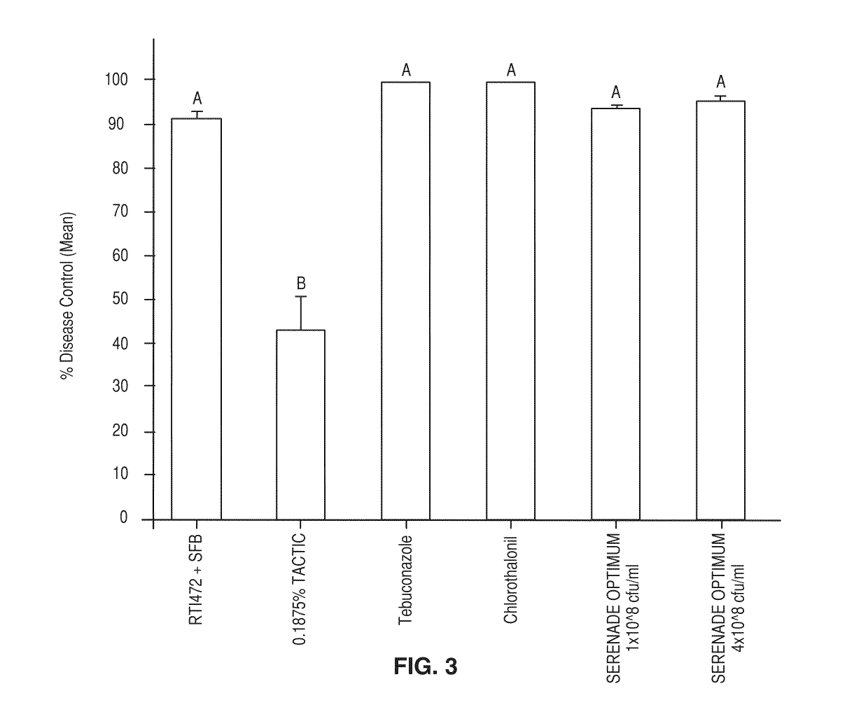

In addition, experiments were performed to determine the growth promoting and antagonisitic activities of the Bacillus amyloliquefaciens RTI472 strain in vitro and in various plants under varying conditions. The experimental results are provided in FIGS. 3-16 and in EXAMPLES 3-16 herein. The experiments show the ability of the Bacillus amyloliquefaciens RTI472 to benefit plant growth and confer protection against or control plant pathogenic infection as compared to commercially available SERENADE (BAYER CROP SCIENCE, INC) that contains as an active ingredient Bacillus subtilis strain QST713. In addition to use as a stand alone application, the Bacillus amyloliquefaciens RTI472 strain was also used in programs to prevent/treat plant pathogenic infection where it was applied to plants in alternating time intervals with one or more commercially available chemical fungicides/bacteriocides. In some cases, application of the Bacillus amyloliquefaciens RTI472 strain alone performed as well as a chemical fungicide/bacteriocide. In some cases the RTI472 strain can be used to replace one of the chemical actives in the program. The RTI472 strain was also effective in increasing yield and reducing disease when used as a corn seed treatment. The experimental results provide an example of the benefits of the new RTI472 strain to enhance the antagonistic properties of FRACTURE (CONSUMO EM VERDE (CEV), BIOTECNOLOGIA DAS PLANTAS S.A., PORTUGAL), a plant extract which contains the BLAD polypeptide as an active ingredient. Indentification of new, antimicrobial metabolites produced by the RTI472 strain is also described.

In one embodiment of the present invention, a composition is provided that includes a biologically pure culture of Bacillus amyloliquefaciens RTI472 deposited as ATCC No. PTA-121166, or a mutant thereof having all the identifying characteristics thereof, for application to a plant for one or both of benefiting plant growth or conferring protection against a pathogenic infection in a susceptible plant.

In another embodiment, a method is provided for one or both of benefiting growth of a plant or conferring protection against pathogenic infection in a susceptible plant, the method including delivering a composition comprising a biologically pure culture of Bacillus amyloliquefaciens RTI472 deposited as ATCC No. PTA-121166, or a mutant thereof having all the identifying characteristics thereof to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit the plant growth and/or to confer protection against pathogenic infection in the susceptible plant.

Beneficial plant associated bacteria, both rhizospheric and endophytic, are known to provide a multitude of benefits to host plants that ranges from resistance to diseases and insects pests and tolerance to environmental stresses including cold, salinity and drought stress. As the plants with inoculated plant growth promoting bacteria aquire more water and nutrient from soils, e.g. due to a better developed root system, the plants grow healthier and are less susceptible to biotic and abiotic stresses. As such the microbial compositions of the present invention can be applied alone or in combination with current crop management inputs such as chemical fertilizers, herbicides, and pesticides to maximize crop productivity. Plant growth promoting effects translate into faster growing plants and increase above ground biomass, a property that can be applied to improve early vigor. One benefit of improved early vigor is that plants are more competitive and out-compete weeds, which directly reduces the cost for weed management by minimizing labor and herbicide application. Plant growth promoting effects also translate into improved root development, including deeper and wider roots with more fine roots that are involved in the uptake of water and nutrients. This property allows for better use of agricultural resources, and a reduction in water used in irrigation needs and/or fertilizer application. Changes in root development and root architecture affect the interactions of the plant with other soil-borne microorganisms, including beneficial fungi and bacteria that help the plant with nutrient uptake including nitrogen fixation and phosphate solubilization. These beneficial microbes also compete against plant pathogens to increase overall plant health and decrease the need for chemical fungicides and pesticides.

The antagonistic properties of the Bacillus amyloliquefaciens RTI472 against several major plant pathogens in plate assays is described in EXAMPLE 3 and phenotypic traits such as phytohormone production, acetoin and indole acetic acid (IAA), and nutrient cycling of the strain are described in EXAMPLE 4. In addition, studies were performed in the greenhouse and in field trials on various crops to determine the ability of the B. amyloliquefaciens RTI472 strain to prevent and/or ameliorate the effects of natural or artificial infection of the plants by a number of common plant pathogens. The results are described in EXAMPLES 5-14 and in FIGS. 3-14.

EXAMPLE 5 describes the ability of the B. amyloliquefaciens RTI472 strain to ameliorate the effects of the plant pathogen bean rust (Uromyces appendiculatus) and the plant pathogen Pepper Botrytis Blight (Botrytis cinerea). In addition, in a first set of experiments, different formulations of the B. amyloliquefaciens RTI472 strain were tested for foliar application of the RTI472 strain to control Uromyces appendiculatus and Botrytis cinerea along with a separate and newly identified B. amyloliquefaciens strain, RTI301. The experimental design was set up such that nine days after infection with the pathogen, the percent of disease control was evaluated for each of: RTI472 spores in Spent Fermentation Broth diluted with water alone (“RTI472+1% SFB”), RTI472 spores in Spent Fermentation Broth diluted with water plus yeast extract (“RTI472+1% SFB+Yeast Extract”), RTI301 spores in Spent Fermentation Broth diluted 100 fold with water alone (“RTI301+1% SFB”), RTI301 spores in Spent Fermentation Broth diluted 100 fold with water plus yeast extract (“RTI301+1% SFB+Yeast Extract”), BRAVO WEATHER STIK (500 g a.i./ha Chlorothalonil), HORIZON (50 g a.i./ha Tebuconazole), and SERENADE OPTIMUM at the same spore concentration as the RTI472 and RTI301 strains. The non-treated control (water only) resulted in 28% disease. The results for the Bean Rust and Pepper Botrytis Blight experiments were similar. The results for the Bean Rust experiment are shown in Table III and indicate that the addition of the yeast extract resulted in about a 40% increase in disease control as compared to the strains applied without the addition of yeast extract. The amount of disease control exhibited by RTI472+1% SFB+Yeast Extract and RTI301+1% SFB+Yeast Extract was similar to that observed for SERENADE OPTIMUM when applied at the same rate (i.e., 1×10⁸ cfu/ml) even though the amount of SFB in the RTI472 and RTI301 formulations was relatively low at 1%, and the SFB can be expected to contain secreted compounds having antifungal activity.

Another similar experiment is described in EXAMPLE 6 for disease control of bean rust by the RTI472 strain and the results are shown in Table IV and FIG. 3. The results show comparable control of Bean Rust (Uromyces appendiculatus) after treatment of the bean plant foliage with RTI472 spores as compared to treatment with SERENADE OPTIMUM when applied at the same rate.

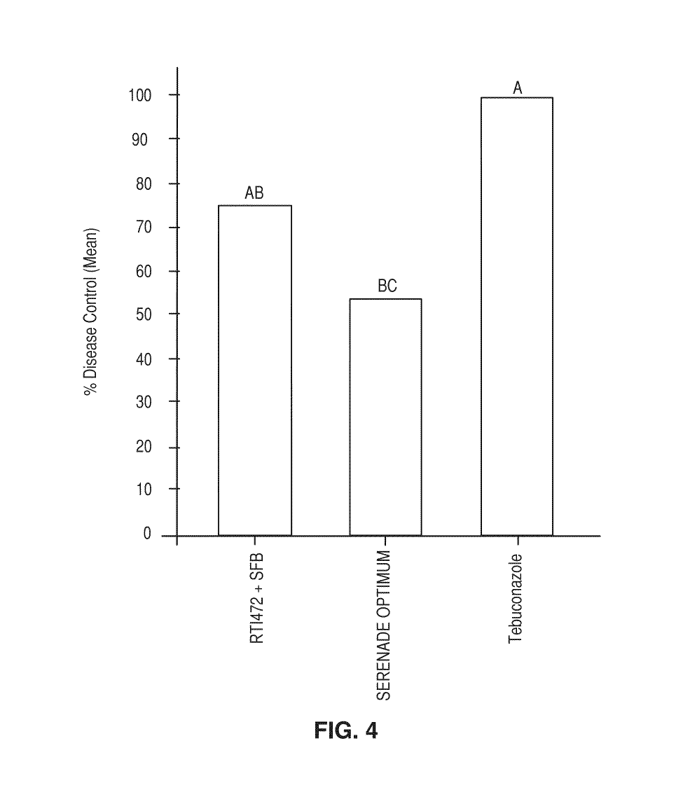

The results in EXAMPLE 6 and Table V and FIG. 4 show improved control of Soybean Powdery Mildew (Microsphaera diffusa) after treatment of the soybean foliage with RTI472 spores as compared to treatment with SERENADE OPTIMUM when applied at the same rate.

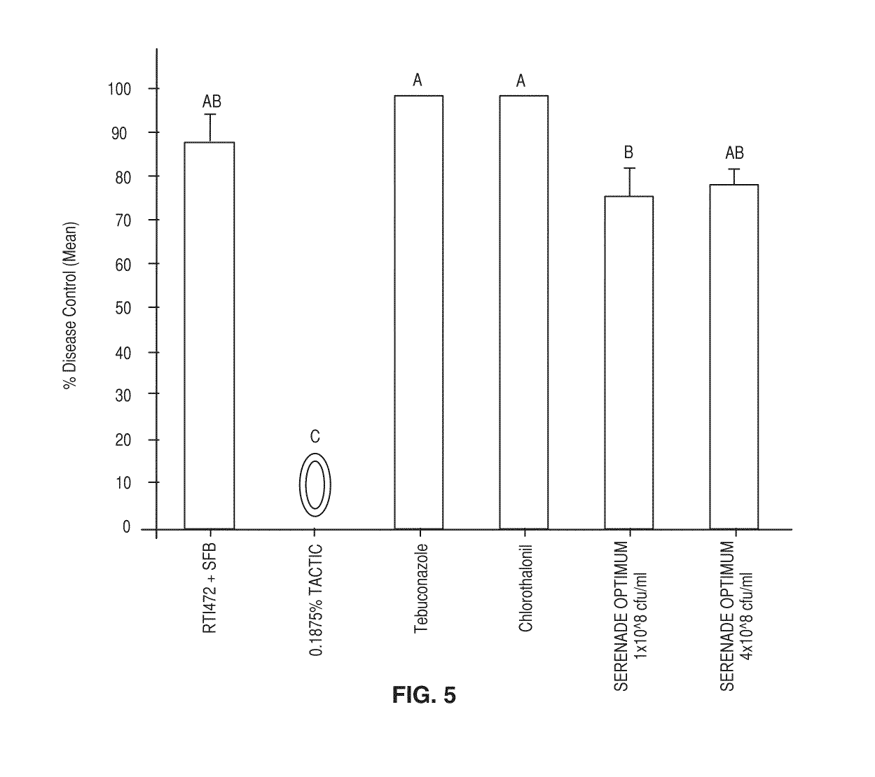

EXAMPLE 7 describes a similar experiment showing the ability of the RTI472 strain to control Pepper Botrytis Blight caused by Botrytis cinerea. The results are shown in Table VI and FIG. 5 and show improved pathogen control after treatment of the pepper plant foliage with spores of RTI472 as compared to treatment of the foliage with SERENADE OPTIMUM at an equal concentration of bacterial spores.

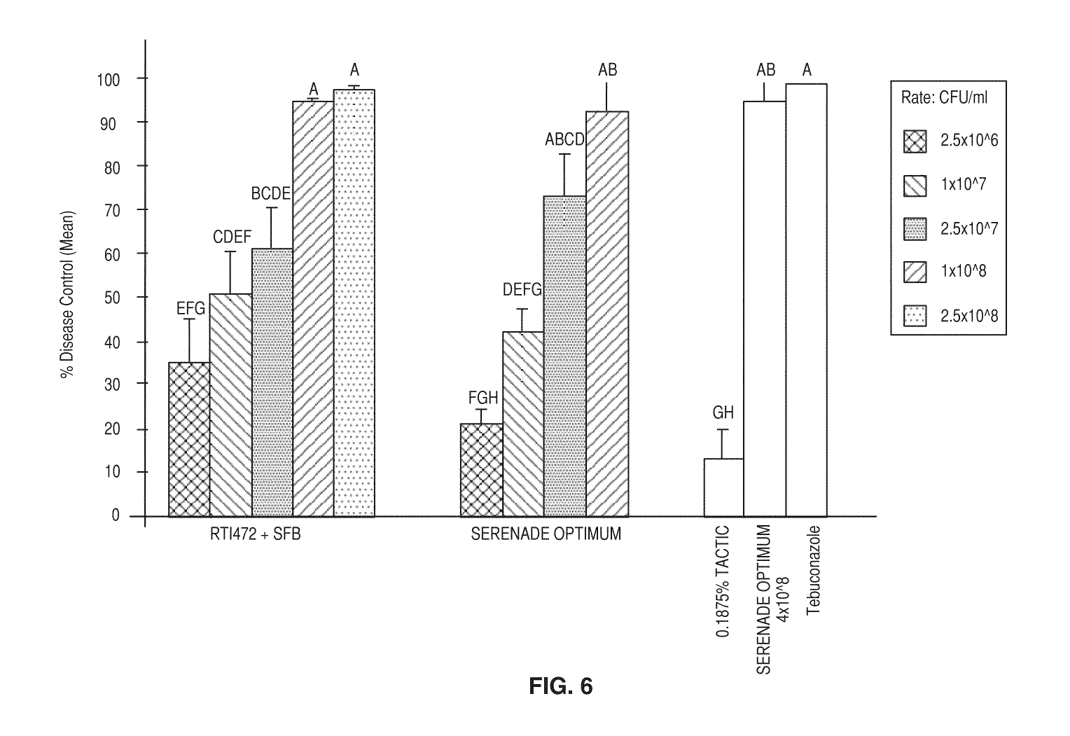

Additional experiments are described in EXAMPLE 7 showing improved control of Pepper Botrytis Blight (Botrytis cinerea) by RTI472 as compared to SERENADE OPTIMUM. The results are shown in Table VII and FIG. 6 and show improved pathogen control by treatment of the pepper plant foliage with increasing concentrations of spores of RTI472 as compared to treatment of the foliage with SERENADE OPTIMUM at equal concentrations of bacterial spores.

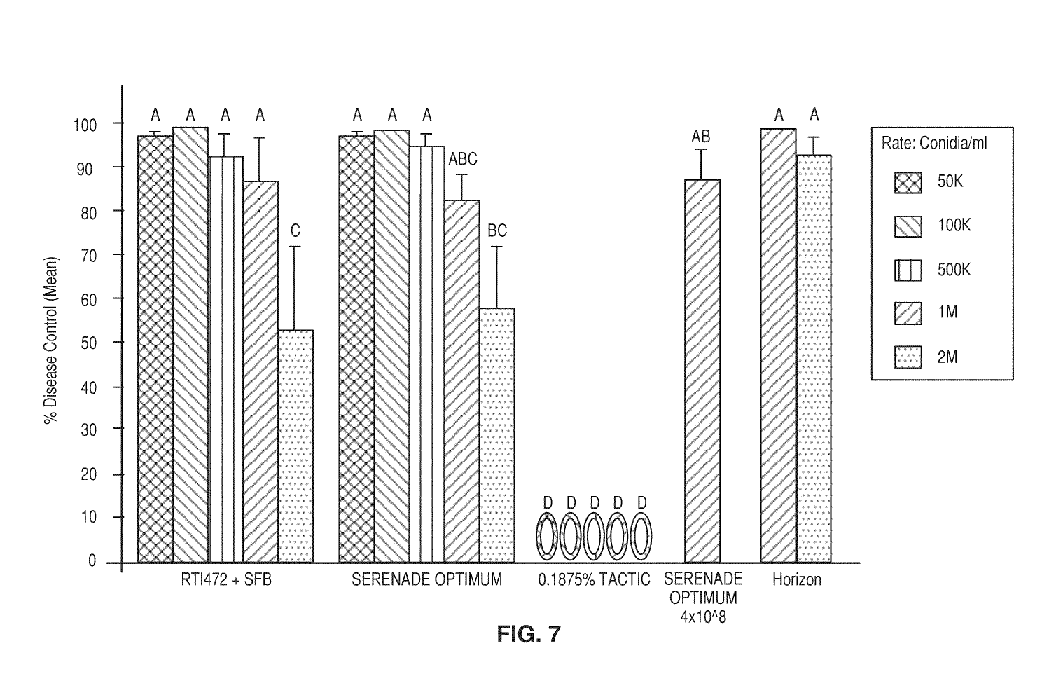

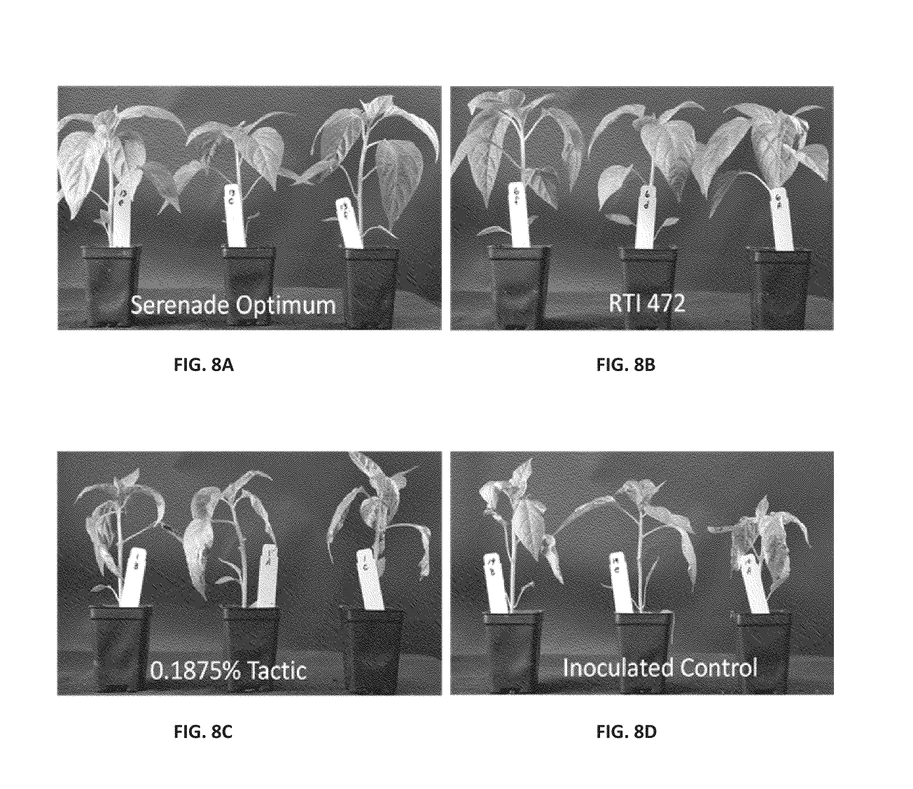

Further results are described in EXAMPLE 7, Table VIII, and FIGS. 7-12 showing control of the pathogen Pepper Botrytis Blight (Botrytis cinerea) by treatment of the pepper plant foliage with spores of RTI472 after increasingly higher doses of inoculation of the plants with the pathogen (50 k, 100 k, 500 k, 1M and 2M conidia/ml) as compared to treatment with product SERENADE OPTIMUM at equal concentration of bacterial spores. FIGS. 8-12 are images of the pepper plants 4 days after infection with the increasing doses of Pepper Botrytis Blight 50 k, 100 k, 500 k, 1M and 2M conidia/ml, respectively, and after treatment of the pepper plant foliage with RTI472 or SERENADE OPTIMUM spores. At the 3 lowest pathogen inoculation rates, a similar percentage of disease control was observed for the RTI472 strain and SERENADE OPTIMUM. However, a statistical improvement was observed for the plants treated with the RTI472 strain as compared to SERENADE OPTIMUM at the 2 highest rates of pathogen inoculation, 1M and 2M.

Studies were performed in field trials on various crops to determine the ability of the B. amyloliquefaciens RTI472 strain to prevent and/or ameliorate the effects of natural or artificial infection of the plants by a number of common plant pathogens. The results are described in EXAMPLES 8-11. In the trials, the RTI472 was applied to the foliage of the crop at the same rate as SERENADE OPTIMUM. The RTI472 spores were applied to the plants either as a stand alone biofungicide or in combination with commercially available chemical fungicides/bacteriocides. Applications were performed 1 to 6 times with 5 to 7 day intervals between applications depending on the crop. The timing of the first application depended on the particular crop and ranged from at the time of planting, a fews weeks after crop emergence, at the time of flowering, upon disease emergence, or prior to expectation of disease emergence.

The results in EXAMPLE 8 and Tables IX-XI show comparable or improved control of powdery mildew in cucurbits by RTI472 as compared to SERENADE OPTIMUM when applied at the same rate as a stand alone biofungicide or as part of a treatment program using commercial products containing chemical fungicides/bacteriocides. RTI472 as a stand-alone biofungicide showed similar performance as the programs using the industry standard Fluopyram plus Tebuconazole fungicide.

The results in EXAMPLE 9 and Tables XII-XIV show comparable or improved control of white mold in snap bean by RTI472 as compared to SERENADE OPTIMUM when applied at the same rate as a stand alone biofungicide or as part of a treatment program using commercial products containing chemical fungicides/bacteriocides. RTI472 as a stand-alone biofungicide showed similar performance as the programs using the industry standard Fluopyram plus Tebuconazole fungicide.

The results in EXAMPLE 9 and Table XII shows comparable or improved control of white mold in snap bean by RTI472 as compared to SERENADE OPTIMUM when applied at the same rate as a stand alone biofungicide or as part of a treatment program using commercial products containing chemical fungicides/bacteriocides. The best control of white mold on snap beans was observed for the program using the combination of B. amyloliquefaciens RTI472 and Thiophanate-methyl fungicide, which was even better than the chemical program based on the use of Prothioconazole fungicide combined with Thiophanate-methyl fungicide.

The results in EXAMPLE 9 and Tables XIII-XIV show improved control of Leaf Spot and Southern White Mold and significant increase in yield in peanut (moderate pressure in Table XIII and heavy pressure in Table XIV) by RTI472 as compared to SERENADE OPTIMUM when applied at the same rate as a stand alone biofungicide.

The results in EXAMPLE 10 and Table XV show improved control of wheat head scab and soybean rust by RTI472 as compared to SERENADE OPTIMUM when applied at the same rate as a stand alone biofungicide. The results in EXAMPLE 10 and Table XV show improved control of Alternaria solani in tomato by RTI472 as compared to control plants when applied as a stand alone biofungicide.

The results in EXAMPLE 11 and Table XVI show improved control of Bacterial Spot Tomato Disease (Xanthomonas) in tomatoes by RTI472 as compared to SERENADE OPTIMUM when applied at the same rate as a stand alone biofungicide or as part of a treatment program using commercial products containing chemical fungicides/bacteriocides. The best control of Bacterial Spot Tomato Disease (Xanthomonas) on tomatoes was observed for B. amyloliquefaciens RTI472 as a stand alone or in the program with copper hydroxide, and outperformed the program based on the use of Chlorothalonil combined with copper hydroxide.

EXAMPLE 12 describes the beneficial effect of coating corn seed with spores of the B. amyloliquefaciens RTI472 strain in addition to a typical combination of chemical active agents (MAXIM+Metalaxyl+PONCHO 250) to improve yield and to control against naturally occurring plant diseases as well as against Rhizoctonia and Fusarium graminearum infections. In a first experiment, the untreated seed and each of the treated corn seed were planted in three separate field trials in Wisconsin and analyzed by length of time to plant emergence, plant stand, plant vigor, and grain yield in bushels/acre. Inclusion of the B. amyloliquefaciens RTI472 in the seed treatment as compared to the seed treated with chemical control alone did not have a statistically significant effect on time to plant emergence, plant stand, or plant vigor, but did result in an increase of 6 bushels/acre of grain (from 231 to 237 bushels/acre) representing a 2.6% increase in grain yield. In a related experiment, when the corn plants in the trial were challenged separately with the pathogens Rhizoctonia and Fusarium graminearum, treatment of the seed with B. amyloliquefaciens RTI472 as compared to seed treated with chemical control alone resulted in a statistically significant decrease in disease severity for both Rhizoctonia and for naturally occurring diseases.

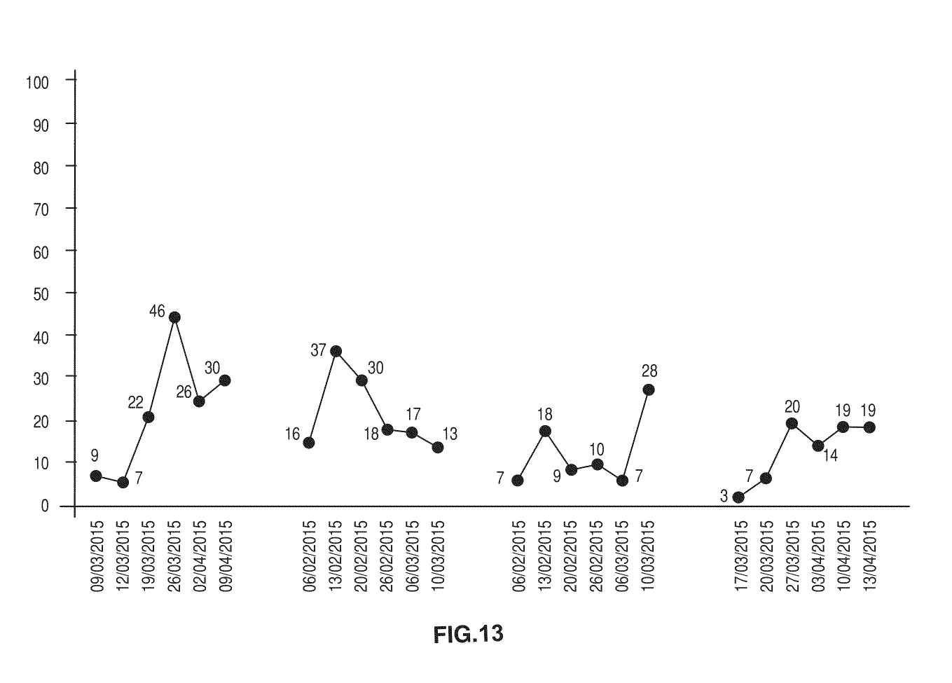

EXAMPLE 13 describes studies performed in field trials of strawberry to determine the ability of the B. amyloliquefaciens RTI472 strain to prevent and/or ameliorate the effects of the plant pathogen Brownish Grey Mildew (Botrytis cinerea). The RTI472 strain was compared to application of a combination of chemical active agents referred to as the “FARMER's program” and application of SERENADE MAX having a 10-folder higher concentration of Bacillus subtilis strain QST713 than the RTI472 strain. The results in Table XVIII show that improved control of Brownish Grey Mildew on strawberry over the untreated control was observed for all three treatments, B. amyloliquefaciens RTI472, SERENADE MAX, and the FARMER's program, with a slightly higher numerical increase of yield for the treatment with RTI472. The development over time of the fruits infected with the Botrytis cinerea pathogen in the untreated control (“UT”) in each of the strawberry trials is shown in the graphs in FIG. 13.

EXAMPLE 14 describes field trials studies of summer squash to determine the ability of the B. amyloliquefaciens RTI472 strain as a stand-alone or as a mixture with the active agent in the product FRACTURE to prevent and/or ameliorate the effects of powdery mildew caused by Golovinomyces cichoracearum (ERYSCI). The results of the experiment are shown in Table XIX. FRACTURE provided approximately 15% reduction of disease severity, while SERENADE OPTIMUM and RTI472 gave approximately 30% reduction of disease severity. The most significant reductions in disease severity were observed for BRAVO WEATHER STIK (45%) and LUNA EXPERIENCE (94%). When RTI472 and FRACTURE were applied together, a cumulative effect for disease control was observed, resulting in a reduction of 47% in disease severity. This is comparable to the reduction in disease severity for BRAVO WEATHER STIK (45%) and demonstrates the usefulness of the combination of B. amyloliquefaciens RTI472 and FRACTURE as an alternative to the chlorothalonil fungicide in a program for the control powdery mildew on cucurbits.

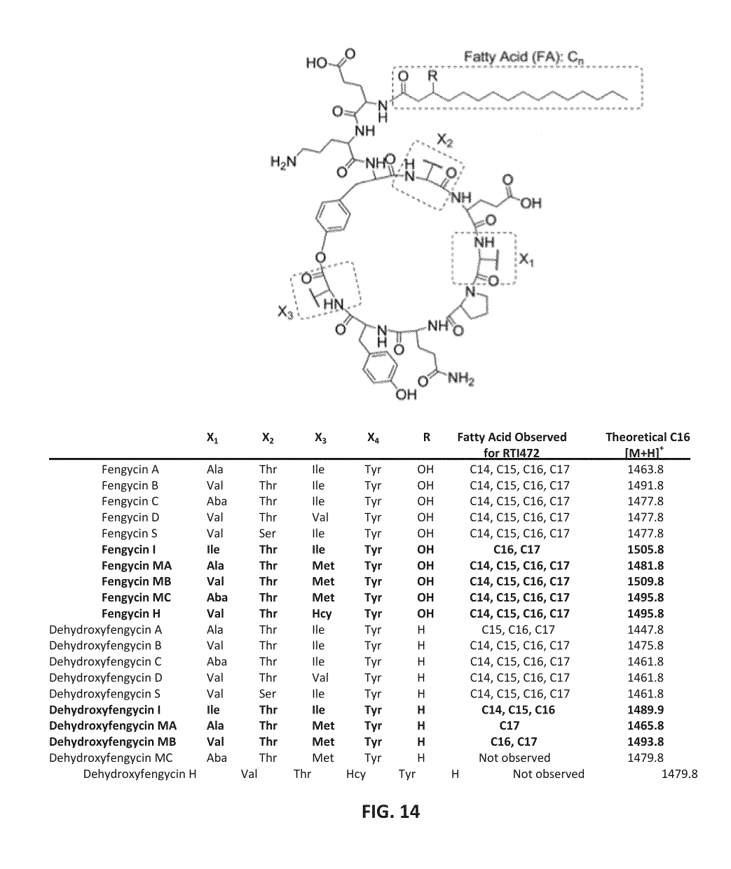

EXAMPLE 15 describes the investigation of the cyclic lipopeptides, Fengycins and Dehydroxyfengycins, produced by the Bacillus amyloliquefaciens RTI472 strain, and surprisingly, the identification of several previously unreported classes of these molecules. It was determined that Bacillus amyloliquefaciens RTI472 produces the previously reported Fengycin A, B and C compounds and the Dehydroxyfengycin A, B and C compounds. Surprisingly, in addition to these known compounds, it was determined that the RTI472 strain also produces previously unidentified derivatives of these compounds where the L-isoleucine at position 8 of the cyclic peptide chain (referred to as X₃ in FIG. 14) is replaced by L-methionine. The new classes of Fengycin and Dehydroxyfengycin are referred to herein as MA, MB and MC, referring to derivatives of classes A, B and C in which the L-isoleucine at X₃ in FIG. 14 has been replaced by L-methionine. The newly identified molecules are shown in FIG. 14 and in Table XX in bold. As noted in Table XX, the Dehydroxyfengycin MC compound was not observed in the culture of the RTI472 strain. It was further determined that the RTI472 strain produces an additional class of Fengycin that has not been previously identified. In this class, the L-isoleucine of Fengycin B (position X₃ in FIG. 14) is replaced by L-homo-cysteine (Hcy). This previously unidentified Fengycin metabolite is referred to herein as Fengycin H and is shown in FIG. 14 and Table XX in bold. It was further determined that the RTI472 strain produces an additional previously unidentified class of Fengycin and Dehydroxyfengycin metabolites. In this class, the amino acid at position 4 of the cyclic peptide backbone structure (position X₁ in FIG. 14) is replaced by L-isoleucine. These previously unidentified metabolites are referred to herein as Fengicin I and Dehydroxyfengicin I and are shown in FIG. 14 and in Table XX.

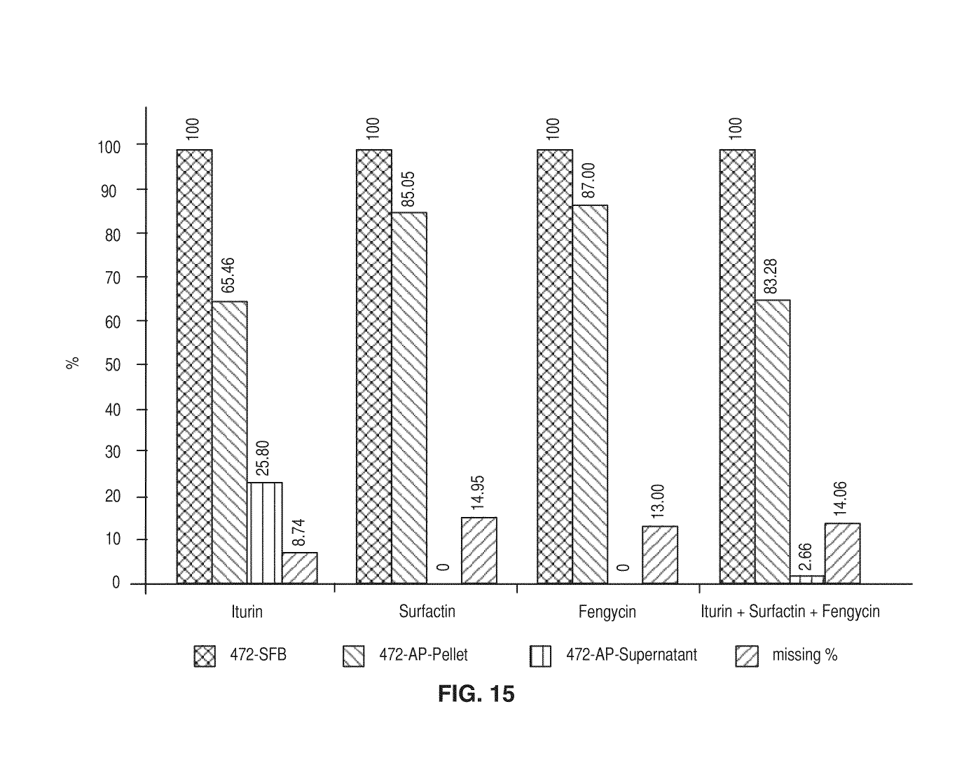

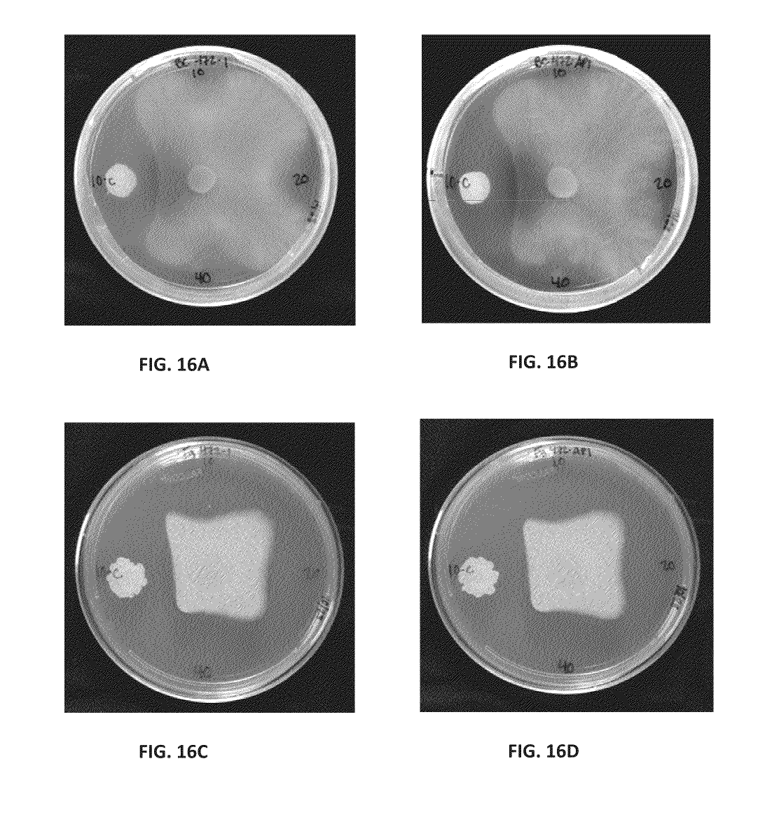

EXAMPLE 16 describes the isolation of antagonistic lipopeptides from B. amyloliquefaciens strain RTI472 spent fermentation broth and an in vitro plate assay showing that the isolated lipopeptides retain their activity against two common plant pathogens. The RTI472 was cultured and an acid precipitate of the culture supernatant was analyzed by LCMS to compare the relative abundance of the iturins, surfactins, and fengycins. FIG. 15 is a graph showing the percentage of recovered lipopeptides from the RTI472 spent fermentation broth after the acid precipitation. The results show that 83% of the total amount of lipopeptides was recovered by acid precipitation. Next, a plate bioassay was performed with the same samples analyzed by LCMS against Botrytis cinerea and Fusarium graminearum. The results are shown in FIGS. 16A-16D and show that the acid precipitated sample has a similar level of antagonistic activity as the starting spent fermentation broth against both Botrytis cinerea and Fusarium graminearum.

In one embodiment, a composition is provided that includes a biologically pure culture of Bacillus amyloliquefaciens RTI472 deposited as ATCC No. PTA-121166, or a mutant thereof having all the identifying characteristics thereof, for application to a plant for one or both of benefiting plant growth or conferring protection against a pathogenic infection in a susceptible plant. The growth benefit of the plant and/or the conferred protection is exhibited by improved seedling vigor, improved root development, improved plant growth, improved plant health, increased yield, improved appearance, improved resistance to plant pathogens, reduced pathogenic infection, or a combination thereof.

The compositions and methods of the present invention are beneficial to a wide range of plants including, but not limited to, monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, sugarcane, and sugarbeet.

In one or more embodiments, the plant can include soybean, bean, snap bean, wheat, cotton, corn, pepper, tomato, potato, cassava, grape, strawberry, banana, peanut, squash, pumpkin, eggplant, and cucumber.

In the compositions and methods of the present invention, the pathogenic infection can be caused by a wide variety of plant pathogens including, for example, but not limited to, a plant fungal pathogen, a plant bacterial pathogen, a rust fungus, a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium colmorum, a Fusarium graminearum, a Fusarium oxysporum, a Fusarium oxysporum f. sp. Cubense, a Fusarium oxysporum f. sp. Lycopersici, a Fusarium virguliforme, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum Candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, and a Helminthosporium tritici-repentis, or combinations thereof.

In some embodiments, the pathogenic infection can be caused by one or a combination of: Soybean rust fungi (Phakopsora pachyrhizi, Phakopsora meibomiae) and the plant comprises soybean; Botrytis cinerea (Botrytis Blight) and the plant comprises grape; Botrytis cinerea (Botrytis Blight) and the plant comprises strawberry; Alternaria spp. (e.g. A. solani) and the plant comprises tomato; Alternaria spp. (e.g. A. solani) and the plant comprises potato; Bean Rust (Uromyces appendiculatus) and the plant comprises common bean; Microsphaera diffusa (Soybean Powdery Mildew) and the plant comprises soybean; Mycosphaerella fijiensis (Black sigatoga) or Fusarium oxysporurn f. sp, cubense (Panama disease) and the plant comprises banana; Xanthomonas spp. or Xanthomonas oryzae pv. oryzae and the plant comprises rice; Xanthomonas axonopodis and the plant comprises cassava; Xanthomonas campestris and the plant comprises tomato; Botrytis cinerea (Pepper Botrytis Blight) and the plant comprises pepper; Powdery mildew and the plant comprises a cucurbit; Sclerotinia sclerotiorum (white mold) and the plant comprises snap bean; Sclerotinia sclerotiorum (white mold) and the plant comprises potato; Sclerotinia homeocarpa (dollar spot) and the plant comprises turfgrass; Southern White Mold and the plant comprises peanut; Leaf spot (Cercospora/Cercosporidium) and the plant comprises peanut; Fusarium graminearum (Wheat Head Scab) and the plant comprises wheat; Mycosphaerella graminicola (Septoria tritici blotch) and the plant comprises wheat; Stagonospora nodorum (glume blotch and septoria nodorum blotch), and the plant compromises wheat; Erwinia amylovora, and the plant compromises apple, pear and other pome fruits; Venturia inaequalis, and the plant compromises apple, pear and other pome fruits; or Rhizoctonia solani and the plant comprises wheat, rice, turfgrass, soybean, corn, legumes and vegetable crops.

The compositions including the RTI472 strain can be in the form of a liquid, an oil dispersion, a dust, a dry wettable powder, a spreadable granule, or a dry wettable granule. The compositions benefit plant growth when applied to foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, when applied in an amount suitable to benefit the plant growth and/or to confer protection against pathogenic infection in the susceptible plant. The compositions can further include one or a combination of a microbial, a biological, or a chemical insecticide, fungicide, nematicide, bacteriocide, herbicide, plant extract, plant growth regulator, or fertilizer present in an amount suitable to benefit plant growth and/or to confer protection against a pathogenic infection in a susceptible plant. The compositions can further include one or a combination of a carrier, a surfactant, a dispersant, or a yeast extract. For the purposes of this specification and claims, the terms “surfactant” and “adjuvant” are used interchangeably. The yeast extract can be delivered at a rate for benefiting plant growth ranging from about 0.01% to 0.2% w/w.

The composition can be in the form of a planting matrix. The planting matrix can be in the form of a potting soil.

In one embodiment, a composition is provided comprising: a biologically pure culture of one or more microorganisms having properties beneficial to one or both of plant growth or plant health; and a yeast extract, for application to a plant for one or both of benefiting plant growth or conferring protection against a pathogenic infection in a susceptible plant. The microorganism can comprise a fungal species. The microorganism can comprise a bacterial species. The microorganism can comprise a Bacillus spp. microorganism. The microorganism can comprise a Bacillus amyloliquefaciens. The composition can further comprise one or a combination of a carrier, a surfactant, or a dispersant. The composition can further comprise one or a combination of a microbial, a biological, or a chemical insecticide, fungicide, nematicide, bacteriocide, herbicide, plant extract, plant growth regulator, or fertilizer.

In one embodiment, a composition is provided for one or both of benefiting plant growth or conferring protection against pathogenic infection in a susceptible plant, the composition including both a biologically pure culture of Bacillus amyloliquefaciens RTI472 deposited as ATCC No. PTA-121166, or a mutant thereof having all the identifying characteristics thereof, in an amount suitable to benefit plant growth and/or to confer protection against pathogenic infection in the susceptible plant; and one or a combination of a microbial, a biological, or a chemical insecticide, fungicide, nematicide, bacteriocide, herbicide, plant extract, plant growth regulator, or fertilizer, in an amount suitable to benefit plant growth and/or to confer protection against pathogenic infection in the susceptible plant. In this embodiment, the biologically pure culture of Bacillus amyloliquefaciens RTI472 and the one or a combination of the microbial, the biological or the chemical insecticide, fungicide, nematicide, bacteriocide, herbicide, plant extract, plant growth regulator, or fertilizer are formulated together.

In one embodiment, the fungicide can include an extract from Lupinus albus. In one embodiment, the fungicide can include a BLAD polypeptide. The BLAD polypeptide can be a fragment of the naturally occurring seed storage protein from sweet lupine (Lupinus albus) that acts on susceptible fungal pathogens by causing damage to the fungal cell wall and disrupting the inner cell membrane. The compositions can include about 20% of the BLAD polypeptide.

In the compositions including Bacillus amyloliquefaciens RTI472, the composition can be in the form of a liquid and the Bacillus amyloliquefaciens RTI472 can be present at a concentration of from about 1.0×10⁸ CFU/ml to about 1.0×10¹² CFU/ml. The composition can be in the form of a dust, a dry wettable powder, a spreadable granule, or a dry wettable granule and the Bacillus amyloliquefaciens RTI472 can be present in an amount of from about 1.0×10⁸ CFU/g to about 1.0×10¹² CFU/g. The composition can be the form of an oil dispersion and the Bacillus amyloliquefaciens RTI472 can be present at a concentration of from about 1.0×10⁸ CFU/ml to about 1.0×10¹² CFU/ml. The Bacillus amyloliquefaciens RTI472 can be in the form of spores or vegetative cells.

In one embodiment, a method is provided for one or both of benefiting growth of a plant or conferring protection against pathogenic infection in a susceptible plant, the method including delivering a composition comprising a biologically pure culture of Bacillus amyloliquefaciens RTI472 deposited as ATCC No. PTA-121166, or a mutant thereof having all the identifying characteristics thereof to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium, in an amount suitable to benefit the plant growth and/or to confer protection against pathogenic infection in the susceptible plant. The composition can be delivered to the foliage of the plant.

In the method, the composition including the Bacillus amyloliquefaciens RTI472 can further include one or a combination of a carrier, a surfactant, a dispersant, or a yeast extract. The yeast extract can be delivered at a rate for benefiting plant growth ranging from about 0.01% to 0.2% w/w.

In another embodiment of the present invention, a method is provided for one or both of benefiting growth of a plant or conferring protection against pathogenic infection in a susceptible plant, the method including delivering a composition comprising a biologically pure culture of Bacillus amyloliquefaciens RTI472 deposited as ATCC No. PTA-121166, or a mutant thereof having all the identifying characteristics thereof, in an amount suitable for benefiting the plant growth and/or conferring protection against the pathogenic infection; and one or a combination of a microbial, a biological, or a chemical insecticide, fungicide, nematicide, bacteriocide, herbicide, plant extract, plant growth regulator, or fertilizer in an amount suitable for benefiting the plant growth and/or conferring protection against the pathogenic infection, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for one or both of benefiting growth of a plant or conferring protection against pathogenic infection in a susceptible plant, the method including delivering a combination of a first composition comprising a biologically pure culture of Bacillus amyloliquefaciens RTI472 deposited as ATCC No. PTA-121166, or a mutant thereof having all the identifying characteristics thereof in an amount suitable to benefit the plant growth and/or to confer protection against pathogenic infection in the susceptible plant; and a second composition comprising one or a combination of a microbial, a biological, or a chemical insecticide, fungicide, nematicide, bacteriocide, herbicide, plant extract, plant growth regulator, or fertilizer, in an amount suitable to benefit the plant growth and/or to confer protection against pathogenic infection in the susceptible plant, to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium. In one embodiment, the first and second compositions can be delivered to the foliage of the plant. The first composition can further include one or a combination of a carrier, a surfactant, a dispersant, or a yeast extract. The yeast extract can be delivered at a rate for benefiting plant growth ranging from about 0.01% to 0.2% w/w.

The fungicide of the second composition can include an extract from Lupinus albus. The fungicide of the second composition can include a BLAD polypeptide. The BLAD polypeptide can be a fragment of the naturally occurring seed storage protein from sweet lupine (Lupinus albus) that acts on susceptible fungal pathogens by causing damage to the fungal cell wall and disrupting the inner cell membrane. The fungicide of the second composition can include about 20% of a BLAD polypeptide.

In the compositions and methods of the present invention for delivering RTI472 in combination with a microbial, a biological, or a chemical insecticide, fungicide, nematicide, bacteriocide, herbicide, plant extract, plant growth regulator, or fertilizer, the growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

In one embodiment, the method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In the methods for delivering RTI472 in combination with a microbial, a biological, or a chemical insecticide, fungicide, nematicide, bacteriocide, herbicide, plant extract, plant growth regulator, or fertilizer, the composition can be in the form of a liquid, an oil dispersion, a dust, a dry wettable powder, a spreadable granule, or a dry wettable granule. The Bacillus amyloliquefaciens RTI472 can be in the form of spores or vegetative cells. The Bacillus amyloliquefaciens RTI472 can be delivered at a rate for benefiting plant growth of about 1.0×10¹⁰ CFU/ha to about 1.0×10¹⁴ CFU/ha. The yeast extract can be delivered at a rate for benefiting plant growth ranging from about 0.01% to 0.2% w/w.

In the compositions and methods of the present invention for delivering RTI472 with a microbial, a biological, or a chemical insecticide, fungicide, nematicide, bacteriocide, herbicide, plant extract, plant growth regulator, or fertilizer, the insecticide can comprise bifenthrin. In one or more embodiments, the nematicide can comprise cadusafos. In one or more embodiments, the insecticide can comprise bifenthrin and the composition can be formulated as a liquid. In one or more embodiments, the insecticide can comprise bifenthrin and clothianidin. In one or more embodiments, the insecticide can comprise bifenthrin and clothianidin and the composition can be formulated as a liquid. In one or more embodiments, the insecticide can comprise bifenthrin or zeta-cypermethrin. In one or more embodiments, the composition can be formulated as a liquid and the insecticide can comprise bifenthrin or zeta-cypermethrin.

The insecticide can be bifenthrin and the composition formulation can further comprise a hydrated aluminum-magnesium silicate, and at least one dispersant selected from the group consisting of a sucrose ester, a lignosulfonate, an alkylpolyglycoside, a naphthalenesulfonic acid formaldehyde condensate and a phosphate ester. The bifenthrin insecticide can be present at a concentration ranging from 0.1 g/ml to 0.2 g/ml. The bifenthrin insecticide can be present at a concentration of about 0.1715 g/ml. The rate of application of the bifenthrin insecticide can be in the range of from about 0.1 gram of bifenthrin per hectare (g ai/ha) to about 1000 g ai/ha, more preferably in a range of from about 1 g ai/ha to about 100 g ai/ha.

In an embodiment, the bifenthrin composition can comprise: bifenthrin; a hydrated aluminum-magnesium silicate; and at least one dispersant selected from a sucrose ester, a lignosulfonate, an alkylpolyglycoside, a naphthalenesulfonic acid formaldehyde condensate and a phosphate ester.

The bifenthrin can be preferably present in a concentration of from 1.0% by weight to 35% by weight, more particularly, from 15% by weight to 25% by weight based upon the total weight of all components in the composition. The bifenthrin insecticide composition can be formulated in a manner suitable for mixture as a liquid with a fertilizer. The bifenthrin insecticide composition can be present in the liquid formulation at a concentration ranging from 0.1 g/ml to 0.2 g/ml. The bifenthrin insecticide may be present in the liquid formulation at a concentration of about 0.1715 g/ml. The terms “can be formulated in a manner suitable for mixture as a liquid with a fertilizer” and “in a formulation compatible with a liquid fertilizer” are herein used interchangeably throughout the specification and claims and are intended to mean that the formulation is capable of dissolution or dispersion or emulsion in an aqueous solution to allow for mixing with a fertilizer for delivery to plants in a liquid formulation.

The dispersant or dispersants can preferably be present in a total concentration of from about 0.02% by weight to about 20% by weight based upon the total weight of all components in the composition.

In some embodiments, the hydrated aluminum-magnesium silicate can be selected from the group consisting of montmorillonite and attapulgite.

In some embodiments, the phosphate ester can be selected from a nonyl phenol phosphate ester and a tridecyl alcohol ethoxylated phosphate potassium salt.

Other embodiments can further include at least one of an anti-freeze agent, an anti-foam agent and a biocide.

In one embodiment a composition is provided for benefiting plant growth, the composition comprising: a biologically pure culture of Bacillus amyloliquefaciens RTI472 deposited as ATCC No. PTA-121166, or a mutant thereof having all the identifying characteristics thereof; and an insecticide. The insecticide can be one or a combination of pyrethroids, bifenthrin, tefluthrin, zeta-cypermethrin, organophosphates, chlorethoxyfos, chlorpyrifos, tebupirimfos, cyfluthrin, fiproles, fipronil, nicotinoids, or clothianidin. The insecticide can include bifenthrin. The insecticide can include bifenthrin and the composition can be in a formulation compatible with a liquid fertilizer. The composition can further include a hydrated aluminum-magnesium silicate and at least one dispersant The bifenthrin insecticide can be present at a concentration ranging from 0.1 g/ml to 0.2 g/ml. The bifenthrin insecticide can be present at a concentration of about 0.1715 g/ml.

In addition, in one or more embodiments, suitable insecticides, herbicides, fungicides, and nematicides of the compositions and methods of the present invention can include the following:

Insecticides: A0) various insecticides, including agrigata, al-phosphide, amblyseius, aphelinus, aphidius, aphidoletes, artimisinin, autographa californica NPV, azocyclotin, Bacillus subtilis, Bacillus thuringiensis- spp. aizawai, Bacillus thuringiensis spp. kurstaki, Bacillus thuringiensis, Beauveria, Beauveria bassiana, betacyfluthrin, biologicals, bisultap, brofluthrinate, bromophos-e, bromopropylate, Bt-Corn-GM, Bt-Soya-GM, capsaicin, cartap, celastrus-extract, chlorantraniliprole, chlorbenzuron, chlorethoxyfos, chlorfluazuron, chlorpyrifos-e, cnidiadin, cryolite, cyanophos, cyantraniliprole, cyhalothrin, cyhexatin, cypermethrin, dacnusa, DCIP, dichloropropene, dicofol, diglyphus, diglyphus+dacnusa, dimethacarb, dithioether, dodecyl-acetate, emamectin, encarsia, EPN, eretmocerus, ethylene-dibromide, eucalyptol, fatty-acids, fatty-acids/salts, fenazaquin, fenobucarb (BPMC), fenpyroximate, flubrocythrinate, flufenzine, formetanate, formothion, furathiocarb, gamma-cyhalothrin, garlic-juice, granulosis-virus, harmonia, heliothis armigera NPV, inactive bacterium, indol-3-ylbutyric acid, iodomethane, iron, isocarbofos, isofenphos, isofenphos-m, isoprocarb, isothioate, kaolin, lindane, liuyangmycin, matrine, mephosfolan, metaldehyde, metarhizium-anisopliae, methamidophos, metolcarb (MTMC), mineral-oil, mirex, m-isothiocyanate, monosultap, myrothecium verrucaria, naled, neochrysocharis formosa, nicotine, nicotinoids, oil, oleic-acid, omethoate, orius, oxymatrine, paecilomyces, paraffin-oil, parathion-e, pasteuria, petroleum-oil, pheromones, phosphorus-acid, photorhabdus, phoxim, phytoseiulus, pirimiphos-e, plant-oil, plutella xylostella GV, polyhedrosis-virus, polyphenol-extracts, potassium-oleate, profenofos, prosuler, prothiofos, pyraclofos, pyrethrins, pyridaphenthion, pyrimidifen, pyriproxifen, quillay-extract, quinomethionate, rape-oil, rotenone, saponin, saponozit, sodium-compounds, sodium-fluosilicate, starch, steinernema, streptomyces, sulfluramid, sulphur, tebupirimfos, tefluthrin, temephos, tetradifon, thiofanox, thiometon, transgenics (e.g., Cry3Bb1), triazamate, trichoderma, trichogramma, triflumuron, verticillium, vertrine, isomeric insecticides (e.g., kappa-bifenthrin, kappa-tefluthrin), dichoromezotiaz, broflanilide, pyraziflumid; A1) the class of carbamates, including aldicarb, alanycarb, benfuracarb, carbaryl, carbofuran, carbosulfan, methiocarb, methomyl, oxamyl, pirimicarb, propoxur and thiodicarb; A2) the class of organophosphates, including acephate, azinphos-ethyl, azinphos-methyl, chlorfenvinphos, chlorpyrifos, chlorpyrifos-methyl, demeton-S-methyl, diazinon, dichlorvos/DDVP, dicrotophos, dimethoate, disulfoton, ethion, fenitrothion, fenthion, isoxathion, malathion, methamidaphos, methidathion, mevinphos, monocrotophos, oxymethoate, oxydemeton-methyl, parathion, parathion-methyl, phenthoate, phorate, phosalone, phosmet, phosphamidon, pirimiphos-methyl, quinalphos, terbufos, tetrachlorvinphos, triazophos and trichlorfon; A3) the class of cyclodiene organochlorine compounds such as endosulfan; A4) the class of fiproles, including ethiprole, fipronil, pyrafluprole and pyriprole; A5) the class of neonicotinoids, including acetamiprid, clothianidin, dinotefuran, imidacloprid, nitenpyram, thiacloprid and thiamethoxam; A6) the class of spinosyns such as spinosad and spinetoram; A7) chloride channel activators from the class of mectins, including abamectin, emamectin benzoate, ivermectin, lepimectin and milbemectin; A8) juvenile hormone mimics such as hydroprene, kinoprene, methoprene, fenoxycarb and pyriproxyfen; A9) selective homopteran feeding blockers such as pymetrozine, flonicamid and pyrifluquinazon; A10) mite growth inhibitors such as clofentezine, hexythiazox and etoxazole; A11) inhibitors of mitochondrial ATP synthase such as diafenthiuron, fenbutatin oxide and propargite; uncouplers of oxidative phosphorylation such as chlorfenapyr; A12) nicotinic acetylcholine receptor channel blockers such as bensultap, cartap hydrochloride, thiocyclam and thiosultap sodium; A13) inhibitors of the chitin biosynthesis type 0 from the benzoylurea class, including bistrifluron, diflubenzuron, flufenoxuron, hexaflumuron, lufenuron, novaluron and teflubenzuron; A14) inhibitors of the chitin biosynthesis type 1 such as buprofezin; A15) moulting disruptors such as cyromazine; A16) ecdyson receptor agonists such as methoxyfenozide, tebufenozide, halofenozide and chromafenozide; A17) octopamin receptor agonists such as amitraz; A18) mitochondrial complex electron transport inhibitors pyridaben, tebufenpyrad, tolfenpyrad, flufenerim, cyenopyrafen, cyflumetofen, hydramethylnon, acequinocyl or fluacrypyrim; A19) voltage-dependent sodium channel blockers such as indoxacarb and metaflumizone; A20) inhibitors of the lipid synthesis such as spirodiclofen, spiromesifen and spirotetramat; A21) ryanodine receptor-modulators from the class of diamides, including flubendiamide, the phthalamide compounds (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, chloranthraniliprole and cy-anthraniliprole; A22) compounds of unknown or uncertain mode of action such as azadirachtin, amidoflumet, bifenazate, fluensulfone, piperonyl butoxide, pyridalyl, sulfoxaflor; or A23) sodium channel modulators from the class of pyrethroids, including acrinathrin, allethrin, bifenthrin, cyfluthrin, lambda-cyhalothrin, cyper-methrin, alpha-cypermethrin, beta-cypermethrin, zeta-cypermethrin, deltamethrin, esfenvalerate, etofenprox, fenpropathrin, fenvalerate, flucythrinate, tau-fluvalinate, permethrin, silafluofen and tralomethrin.

Fungicides: B0) benzovindiflupyr, anitiperonosporic, ametoctradin, amisulbrom, copper salts (e.g., copper hydroxide, copper oxychloride, copper sulfate, copper persulfate), boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens (e.g., strains QST 713, FZB24, MB1600, D747), extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, estract from Reynoutria sachalinensis, phosphorous acid and salts, teclofthalam, triazoxide, pyriofenone, organic oils, potassium bicarbonate, chlorothalonil, fluoroimide; B1) azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; B2) strobilurins, including azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; B3) carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1 -methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; B4) heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloro pyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; B5) carbamates, including mancozeb, maneb, metam, methasulphocarb, metiram, ferbam, propineb, thiram, zineb, ziram, diethofencarb, iprovalicarb, benthiavalicarb, propamocarb, propamocarbhydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; or B6) other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

Herbicides: C1) acetyl-CoA carboxylase inhibitors (ACC), for example cyclohexenone oxime ethers, such as alloxydim, clethodim, cloproxydim, cycloxydim, sethoxydim, tralkoxydim, butroxydim, clefoxydim or tepraloxydim; phenoxyphenoxypropionic esters, such as clodinafop-propargyl, cyhalofop-butyl, diclofop-methyl, fenoxaprop-ethyl, fenoxaprop-P-ethyl, fenthiapropethyl, fluazifop-butyl, fluazifop-P-butyl, haloxyfop-ethoxyethyl, haloxyfop-methyl, haloxyfop-P-methyl, isoxapyrifop, propaquizafop, quizalofop-ethyl, quizalofop-P-ethyl or quizalofop-tefuryl; or arylaminopropionic acids, such as flamprop-methyl or flamprop-isopropyl; C2 acetolactate synthase inhibitors (ALS), for example imidazolinones, such as imazapyr, imazaquin, imazamethabenz-methyl (imazame), imazamox, imazapic or imazethapyr; pyrimidyl ethers, such as pyrithiobac-acid, pyrithiobac-sodium, bispyribac-sodium. KIH-6127 or pyribenzoxym; sulfonamides, such as florasulam, flumetsulam or metosulam; or sulfonylureas, such as amidosulfuron, azimsulfuron, bensulfuron-methyl, chlorimuron-ethyl, chlorsulfuron, cinosulfuron, cyclosulfamuron, ethametsulfuron-methyl, ethoxysulfuron, flazasulfuron, halosulfuron-methyl, imazosulfuron, metsulfuron-methyl, nicosulfuron, primisulfuron-methyl, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron, sulfosulfuron, fora msulfuron or iodosulfuron; C3) amides, for example allidochlor (CDAA), benzoylprop-ethyl, bromobutide, chiorthiamid. diphenamid, etobenzanidibenzchlomet), fluthiamide, fosamin or monalide; C4) auxin herbicides, for example pyridinecarboxylic acids, such as clopyralid or picloram; or 2,4-D or benazolin; C5) auxin transport inhibitors, for example naptalame or diflufenzopyr; C6) carotenoid biosynthesis inhibitors, for example benzofenap, clomazone (dimethazone), diflufenican, fluorochloridone, fluridone, pyrazolynate, pyrazoxyfen, isoxaflutole, isoxachlortole, mesotrione, sulcotrione (chlormesulone), ketospiradox, flurtamone, norflurazon or amitrol; C7) enolpyruvylshikimate-3-phosphate synthase inhibitors (EPSPS), for example glyphosate or sulfosate; C8) glutamine synthetase inhibitors, for example bilanafos (bialaphos) or glufosinate-ammonium; C9) lipid biosynthesis inhibitors, for example anilides, such as anilofos or mefenacet; chloroacetanilides, such as dimethenamid, S-dimethenamid, acetochlor, alachlor, butachlor, butenachlor, diethatyl-ethyl, dimethachlor, metazachlor, metolachlor, S-metolachlor, pretilachlor, propachlor, prynachlor, terbuchlor, thenylchlor or xylachlor; thioureas, such as butylate, cycloate, di-allate, dimepiperate, EPTC. esprocarb, molinate, pebulate, prosulfocarb, thiobencarb (benthiocarb), tri-allate or vemolate; or benfuresate or perfluidone; C10) mitosis inhibitors, for example carbamates, such as asulam, carbetamid, chlorpropham, orbencarb, pronamid (propyzamid), propham or tiocarbazil; dinitroanilines, such as benefin, butralin, dinitramin, ethalfluralin, fluchloralin, oryzalin, pendimethalin, prodiamine or trifluralin; pyridines, such as dithiopyr or thiazopyr; or butamifos, chlorthal-dimethyl (DCPA) or maleic hydrazide; C11) protoporphyrinogen IX oxidase inhibitors, for example diphenyl ethers, such as acifluorfen, acifluorfen-sodium, aclonifen, bifenox, chlomitrofen (CNP), ethoxyfen, fluorodifen, fluoroglycofen-ethyl, fomesafen, furyloxyfen, lactofen, nitrofen, nitrofluorfen or oxyfluorfen; oxadiazoles, such as oxadiargyl or oxadiazon; cyclic imides, such as azafenidin, butafenacil, carfentrazone-ethyl, cinidon-ethyl, flumiclorac-pentyl, flumioxazin, flumipropyn, flupropacil, fluthiacet-methyl, sulfentrazone or thidiazimin; or pyrazoles, such as ET-751.JV 485 or nipyraclofen; C12) photosynthesis inhibitors, for example propanil, pyridate or pyridafol; benzothiadiazinones, such as bentazone; dinitrophenols, for example bromofenoxim, dinoseb, dinoseb-acetate, dinoterb or DNOC; dipyridylenes, such as cyperquat-chloride, difenzoquat-methylsulfate, diquat or paraquat-dichloride; ureas, such as chlorbromuron, chlorotoluron, difenoxuron, dimefuron, diuron, ethidimuron, fenuron, fluometuron, isoproturon, isouron, linuron, methabenzthiazuron, methazole, metobenzuron, metoxuron, monolinuron, neburon, siduron or tebuthiuron; phenols, such as bromoxynil or ioxynil; chloridazon; triazines, such as ametryn, atrazine, cyanazine, desmein, dimethamethryn, hexazinone, prometon, prometryn, propazine, simazine, simetryn, terbumeton, terbutryn, terbutylazine or trietazine; triazinones, such as metamitron or metribuzin; uracils, such as bromacil, lenacil or terbacil; or biscarbamates, such as desmedipham or phenmedipham; C13) synergists, for example oxiranes, such as tridiphane; C14) CIS cell wall synthesis inhibitors, for example isoxaben or dichlobenil; C15) various other herbicides, for example dichloropropionic acids, such as dalapon; dihydrobenzofurans, such as ethofumesate; phenylacetic acids, such as chlorfenac (fenac); or aziprotryn, barban, bensulide, benzthiazuron, benzofluor, buminafos, buthidazole, buturon, cafenstrole, chlorbufam, chlorfenprop-methyl, chloroxuron, cinmethylin, cumyluron, cycluron, cyprazine, cyprazole, dibenzyluron, dipropetryn, dymron, eglinazin-ethyl, endothall, ethiozin, flucabazone, fluorbentranil, flupoxam, isocarbamid, isopropalin, karbutilate, mefluidide, monuron, napropamide, napropanilide, nitralin, oxaciclomefone, phenisopham, piperophos, procyazine, profluralin, pyributicarb, secbumeton, sulfallate (CDEC), terbucarb, triaziflam, triazofenamid or trimeturon; or their environmentally compatible salts.

Nematicides or bionematicides: Benomyl, cloethocarb, aldoxycarb, tirpate, diamidafos, fenamiphos, cadusafos, dichlofenthion, ethoprophos, fensulfothion, fosthiazate, heterophos, isamidofof, isazofos, phosphocarb, thionazin, imicyafos, mecarphon, acetoprole, benclothiaz, chloropicrin, dazomet, fluensulfone, 1,3-dichloropropene (telone), dimethyl disulfide, metam sodium, metam potassium, metam salt (all MITC generators), methyl bromide, biological soil amendments (e.g., mustard seeds, mustard seed extracts), steam fumigation of soil, allyl isothiocyanate (AITC), dimethyl sulfate, furfual (aldehyde).

Suitable plant growth regulators of the present invention include the following: Plant Growth Regulators: D1) Antiauxins, such as clofibric acid, 2,3,5-tri-iodobenzoic acid; D2) Auxins such as 4-CPA, 2,4-D, 2,4-DB, 2,4-DEP, dichlorprop, fenoprop , IAA ,IBA, naphthaleneacetamide, α-naphthaleneacetic acids, 1-naphthol, naphthoxyacetic acids, potassium naphthenate, sodium naphthenate, 2,4,5-T; D3) cytokinins, such as 2iP, benzyladenine, 4-hydroxyphenethyl alcohol, kinetin, zeatin; D4) defoliants, such as calcium cyanamide, dimethipin, endothal, ethephon, merphos, metoxuron, pentachlorophenol, thidiazuron, tribufos; D5) ethylene inhibitors, such as aviglycine, 1-methylcyclopropene; D6) ethylene releasers, such as ACC, etacelasil,ethephon, glyoxime; D7) gametocides, such as fenridazon, maleic hydrazide; D8) gibberellins, such as gibberellins, gibberellic acid; D9) growth inhibitors, such as abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid; D10) morphactins, such as chlorfluren, chlorflurenol, dichlorflurenol, flurenol; D11) growth retardants, such as chlormequat, daminozide, flurprimidol, mefluidide, paclobutrazol, tetcyclacis, uniconazole; D12) growth stimulators, such as brassinolide, brassinolide-ethyl, DCPTA, forchlorfenuron, hymexazol, prosuler, triacontanol; D13) unclassified plant growth regulators, such as bachmedesh, benzofluor, buminafos, carvone, choline chloride, ciobutide, clofencet, cyanamide, cyclanilide, cycloheximide, cyprosulfamide, epocholeone, ethychlozate, ethylene, fuphenthiourea, furalane, heptopargil, holosulf, inabenfide, karetazan, lead arsenate, methasulfocarb, prohexadione, pydanon, sintofen, triapenthenol, trinexapac.

The fertilizer can be a liquid fertilizer. The term “liquid fertilizer” refers to a fertilizer in a fluid or liquid form containing various ratios of nitrogen, phosphorous and potassium (for example, but not limited to, 10% nitrogen, 34% phosphorous and 0% potassium) and micronutrients, commonly known as starter fertilizers that are high in phosphorus and promote rapid and vigorous root growth.

Chemical formulations of the present invention can be in any appropriate conventional form, for example an emulsion concentrate (EC), a suspension concentrate (SC), a suspo-emulsion (SE), a capsule suspension (CS), a water dispersible granule (WG), an emulsifiable granule (EG), a water in oil emulsion (EO), an oil in water emulsion (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a dispersible concentrate (DC), a wettable powder (WP) or any technically feasible formulation in combination with agriculturally acceptable adjuvants.

In another embodiment of the present invention, a plant seed is provided that is coated with a composition including spores of a biologically pure culture of Bacillus amyloliquefaciens RTI472 deposited as ATCC No. PTA-121166, or a mutant thereof having all the identifying characteristics thereof, present in an amount suitable to benefit plant growth and/or to confer protection against a pathogenic infection in a susceptible plant. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

The composition coated on the plant seed can include an amount of Bacillus amyloliquefaciens spores from about 1.0×10² CFU/seed to about 1.0×10⁹ CFU/seed.

The plant seed can include, but is not limited to, the seed of monocots, dicots, Cereals, Corn, Sweet Corn, Popcorn, Seed Corn, Silage Corn, Field Corn, Rice, Wheat, Barley, Sorghum, Brassica Vegetables, Broccoli, Cabbage, Cauliflower, Brussels Sprouts, Collards, Kale, Mustard Greens, Kohlrabi, Bulb Vegetables, Onion, Garlic, Shallots, Fruiting Vegetables, Pepper, Tomato, Eggplant, Ground Cherry, Tomatillo, Okra, Grape, Herbs/ Spices, Cucurbit Vegetables, Cucumber, Cantaloupe, Melon, Muskmelon, Squash, Watermelon, Pumpkin, Eggplant, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Cotton, Flax, Peanut, Rapeseed, Safflower, Sesame, Sunflower, Soybean, Root/Tuber and Corm Vegetables, Carrot, Potato, Sweet Potato, Beets, Ginger, Horseradish, Radish, Ginseng, Turnip, sugarcane, sugarbeet, Grass, or Turf grass.

In one or more embodiments, the plant seed can include seed of a drybean, a corn, a wheat, a soybean, a canola, a rice, a cucumber, a pepper, a tomato, a squash, a cotton, a grass, and a turf grass.

The pathogenic infection treated by the coated plant seed can be caused by a plant pathogen including, for example, but not limited to a plant fungal pathogen, a plant bacterial pathogen, a Botrytis spp., a Botrytis cinerea, a Botrytis squamosa, an Erwinia spp., an Erwinia carotovora, an Erwinia amylovora, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Pseudomonas spp., a Pseudomonas syringae pv. Tomato, a Phytophthora spp., a Phytophthora infestans, a Fusarium spp., a Fusarium colmorum, a Fusarium graminearum, a Fusarium oxysporum, a Fusarium oxysporum f. sp. Cubense, a Fusarium oxysporum f. sp. Lycopersici, a Fusarium virguliforme, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Pythium spp., a Pythium ultimum, a Pythium aphanidermatum, a Pythium irregularum, a Pythium ulosum, a Pythium lutriarium, a Pythium sylvatium, a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., or combinations thereof.

The composition coated onto the plant seed can further include one or a combination of a microbial, a biological, or a chemical insecticide, fungicide, nematicide, bacteriocide, or plant growth regulator present in an amount suitable to benefit plant growth and/or to confer protection against a pathogenic infection in a susceptible plant. The insecticide can include bifenthrin. The nematicide can include cadusafos. The insecticide can include bifenthrin and clothianidin.

In another embodiment of the present invention, a method is provided for one or both of benefiting growth of a plant or conferring protection against pathogenic infection in a susceptible plant, the method including planting a seed of the plant or regenerating a vegetative cutting/tissue of the plant in a suitable growth medium, wherein the seed has been coated or the vegetative cutting/tissue has been inoculated with a composition comprising a biologically pure culture of Bacillus amyloliquefaciens RTI472 deposited as ATCC PTA-121166, or a mutant thereof having all the identifying characteristics thereof, wherein growth of the plant from the seed or the vegetative cutting/tissue is benefited and/or protection against pathogenic infection is conferred. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

In one embodiment, the method can further include applying a liquid fertilizer to: soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In one embodiment, a method is provided for benefiting plant growth by conferring protection against or reducing pathogenic infection in a susceptible plant while minimizing the build-up of resistance against the treatment. The method includes delivering to the susceptible plant in separate applications and in altering time intervals a first composition and a second composition, wherein each of the first and second compositions are delivered in an amount suitable to to confer protection against or reduce pathogenic infection in the plant. The first composition includes a biologically pure culture of Bacillus amyloliquefaciens RTI472 deposited as ATCC No. PTA-121166, or a mutant thereof having all the identifying characteristics thereof. The second composition includes one or more chemical active agents having fungicidal or a bacteriocidal properties. In the method the first and second compositions are delivered in the altering time intervals to one or a combination of foliage of the plant, bark of the plant, fruit of the plant, flowers of the plant, seed of the plant, roots of the plant, a cutting of the plant, a graft of the plant, callus tissue of the plant, or soil or growth medium surrounding the plant. In the method, the total amount of the chemical active agent(s) required to confer protection against and/or reduce the pathogenic infection is decreased and the build-up of resistance against the treatment is minimized. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

The first composition can further include one or a combination of a carrier, a surfactant, a dispersant, or a yeast extract. The yeast extract can be delivered at a rate for benefiting plant growth ranging from about 0.01% to 0.2% w/w.

In the method for benefiting plant growth by conferring protection against or reducing pathogenic infection in a susceptible plant while minimizing the build-up of resistance against the treatment, the altering time intervals can range from 1 day to 10 days apart and can be 5 to 7 days apart. The timing of the first application can depend on the particular crop and can range from at the time of planting, a fews weeks after crop emergence, at the time of flowering, upon disease emergence, or prior to expectation of disease emergence. Each of the first and the second compositions can be delivered to the foliage of the plant, the fruit of the plant, or the flowers of the plant. The amount delivered that is suitable to confer protection against or reduce pathogenic infection in the plant can be from about 1.0×10¹⁰ CFU/ha to about 1.0×10¹⁴ CFU/ha Bacillus amyloliquefaciens RTI472 and about 0.01% to 0.2% w/w yeast extract.

The one or more chemical active agents for delivering to the susceptible plant in separate applications and in altering time intervals can include, for example, but are not limited to one or a combination of strobilurine, a triazole, flutriafol, tebuconazole, prothiaconazole, expoxyconazole, fluopyram, chlorothalonil, thiophanate-methyl, Copper Hydroxide fungicide, an EDBC-based fungicide, mancozeb, a succinase dehydrogenase (SDHI) fungicide, bixafen, iprodione, dimethomorph, or valifenalate.

The one or more chemical active agents for delivering to the susceptible plant in separate applications and in altering time intervals can include Fluopyram plus Tebuconazole and delivery of the first composition comprising the RTI472 can replace the delivery of the Chlorothalonil fungicide. The plant can be a cucurbit and the pathogenic infection can be caused by Powdery mildew.

The one or more chemical active agents for delivering to the susceptible plant in separate applications and in altering time intervals can include Thiophanate-methyl fungicide and delivery of the first composition comprising the RTI472 can replace the delivery of a Prothioconazole fungicide.

The one or more chemical active agents for delivering to the susceptible plant in separate applications and in altering time intervals can include copper hydroxide fungicide and delivery of the first composition comprising the RTI472 can replace the delivery of a chlorothalonil fungicide.

In one embodiment, a product is provided including a first component comprising a biologically pure culture of Bacillus amyloliquefaciens RTI472 deposited as ATCC No. PTA-121166, or a mutant thereof having all the identifying characteristics thereof; a second component comprising one or a combination of a microbial, a biological, or a chemical insecticide, fungicide, nematicide, bacteriocide, herbicide, plant extract, plant growth regulator, or fertilizer, wherein the first and second components are separately packaged, and wherein each component is in an amount suitable for one or both of benefiting plant growth or conferring protection against a pathogenic infection in a susceptible plant; and instructions for delivering in an amount suitable to benefit plant growth, a combination of the first and second compositions to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

In the product, the insecticide can be one or a combination of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

In the product, the first composition can further include one or a combination of a carrier, a surfactant, a dispersant, or a yeast extract.

In the product, the first and second compositions can be in the form of a liquid, a dust, a spreadable granule, a dry wettable powder, or a dry wettable granule. In one embodiment, the first composition is in the form of a liquid and the Bacillus amyloliquefaciens RTI472 is present at a concentration of from about 1.0×10⁸ CFU/ml to about 1.0×10¹² CFU/ml. In one embodiment, the first composition is in the form of a dust, a dry wettable powder, a spreadable granule, or a dry wettable granule and the Bacillus amyloliquefaciens RTI472 is present in an amount of from about 1.0×10⁸ CFU/g to about 1.0×10¹² CFU/g. In one embodiment, the first composition is in the form of an oil dispersion and the Bacillus amyloliquefaciens RTI472 is present at a concentration of from about 1.0×10⁸ CFU/ml to about 1.0×10¹² CFU/ml.

In one embodiment of the present invention, a composition is provided, the composition including at least one of an isolated Fengycin-MA compound, an isolated Fengycin MB compound, an isolated Fengycin MC compound, an isolated Dehydroxyfengycin MA compound, an isolated Dehydroxyfengycin MB compound, an isolated Fengycin H compound, an isolated Fengycin I compound, and an isolated Dehyroxyfengycin I compound in an amount suitable to confer protection against a pathogenic infection in a susceptible plant, the compound having the formula:

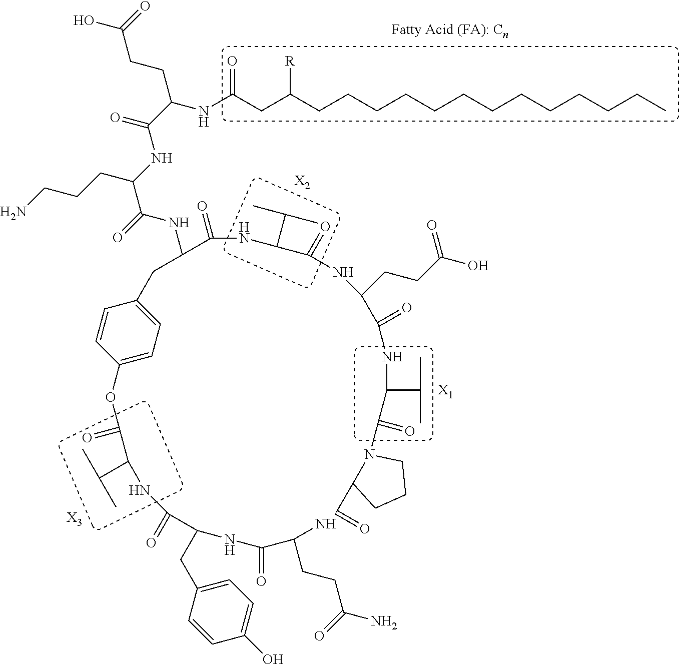

wherein R is OH, n ranges from 8 to 20, FA is linear, iso, or anteiso, and: X₁ is Ala, X₂ is Thr, and X₃ is Met for Fengycin MA; X₁ is Val, X₂ is Thr, and X₃ is Met for Fengycin MB; X₁ is Aba, X₂ is Thr, and X₃ is Met for Fengycin MC; X₁ is Val, X₂ is Thr, and X₃ is Hcy for Fengycin H; and X₁ is Ile, X₂ is Thr, and X₃ is Ile for Fengycin II; and
wherein R is H, n ranges from 8 to 20, FA is linear, iso, or anteiso and: X₁ is Ala, X₂ is Thr, and X₃ is Met for Dehydroxyfengycin MA; X₁ is Val, X₂ is Thr, and X₃ is Met for Dehydroxyfengycin MB; and X₁ is Ile, X₂ is Thr, and X₃ is Ile for Dehydroxyfengycin I.

In another embodiment, the composition further comprises one or a combination of additional isolated Fengycin-and Dehydroxyfengycin-like compounds listed in Table XVI in an amount suitable to confer one or both of a growth benefit on the plant or protection against a pathogenic infection in the susceptible plant.

The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

The Fengycin-MA, -MB, -MC, -H, and -I compounds and the Dehydroxyfengycin-MA, -MB, and -I compounds and one or a combination of additional Fengycin-and Dehydroxyfengycin-like compounds can be isolated by first culturing the RTI472 Bacillus amyloliquefaciens strain, or another Bacillus amyloliquefaciens strain that produces the Fengycin-MA, -MB, -MC, -H, and -I compounds and the Dehydroxyfengycin-MA, -MB, and -I compounds, under suitable conditions well known to those of skill in the art, such as, for example, those conditions described in the EXAMPLES herein, including, but not limited to, culturing the strain for 3 to 6 days in 869 or M2 media. The Fengycin-like and Dehydroxyfengycin-like cyclic lipopeptides present in the Bacillus amyloliquefaciens culture supernatant can then be further isolated using methods well known to those of skill in the art. For example, the Bacillus amyloliquefaciens culture supernatant can be acidified to pH 2 as described herein at EXAMPLE 16, or treated with CaCl₂ (Ajesh, K et al., 2013, “Purification and characterization of antifungal lipopeptide from a soil isolated strain of Bacillus cereus.” In: Worldwide research efforts in the fighting against microbial pathogens: from basic research to technological developments. A. Mendez-Vilas (editor). pp: 227-231) or NH₄SO₄ (Kim, S H et al., 2000, Biotechnol Appl Biochem. 31 (Pt 3):249-253) with or without combining this with an organic extraction step (Kim, P I et al., 2004,J Appl Microbiol. 97(5): 942-949) such as various forms of phase separation including but not limited to direct liquid partitioning, membrane ultrafiltration, and foam fractionation (Baker, S C et al., 2010, Adv Exp Med Biol. 672:281-288).

In one embodiment, the Fengycin-MA, -MB, -MC, -H, and -I compounds and the Dehydroxyfengycin-MA, -MB, and -I compounds and the one or a combination of additional Fengycin-and Dehydroxyfengycin-like compounds listed in Table XVII can be isolated from a biologically pure culture of a Bacillus amyloliquefaciens strain that can produce these compounds.

In one embodiment, the Fengycin-MA, -MB, -MC, -H, and -I compounds and the Dehydroxyfengycin-MA, -MB, and -I compounds and the one or a combination of additional Fengycin-and Dehydroxyfengycin-like compounds listed in Table XVII can be isolated from a biologically pure culture of Bacillus amyloliquefaciens RTI472 deposited as ATCC No. PTA-121166.

In one embodiment, an extract is provided of a biologically pure culture of a Bacillus amyloliquefaciens strain, the extract including a Fengycin-MA, -MB, -MC, -H, and -I compound and a Dehydroxyfengycin-MA, -MB, and -I compound and one or a combination of additional Fengycin- and Dehydroxyfengycin-like compounds listed in Table XVII.

In one embodiment, an extract is provided of a biologically pure culture of Bacillus amyloliquefaciens RTI472 deposited as ATCC No. PTA-121166, the extract including a Fengycin-MA, -MB, -MC, -H, and -I compound and a Dehydroxyfengycin-MA, -MB, and -I compound and one or a combination of additional Fengycin-and Dehydroxyfengycin-like compounds listed in Table XVII.

In one embodiment, the isolated Ericin S compound having molecular weight equal to 3341.6 Da and the isolated Ericin A compound having molecular weight equal to 2985.4 Da and the one or a combination of additional Fengycin- and Dehydroxyfengycin-like compounds listed in Table XVII can be isolated from a biologically pure culture of a Bacillus amyloliquefaciens strain that can produce these compounds.

In one embodiment, the isolated Ericin S compound having molecular weight equal to 3341.6 Da and the isolated Ericin A compound having molecular weight equal to 2985.4 Da and the one or a combination of additional Fengycin- and Dehydroxyfengycin-like compounds listed in Table XVII can be isolated from a biologically pure culture of Bacillus amyloliquefaciens RTI472 deposited as ATCC No. PTA-121166.

In one embodiment, an extract is provided of a biologically pure culture of Bacillus amyloliquefaciens, the extract including at least one of an isolated Ericin S compound having molecular weight equal to 3341.6 Da or an isolated Ericin A compound having molecular weight equal to 2985.4 Da.

In one embodiment, an extract is provided of a biologically pure culture of Bacillus amyloliquefaciens RTI472 deposited as ATCC No. PTA-121166, the extract including at least one of an isolated Ericin S compound having molecular weight equal to 3341.6 Da or an isolated Ericin A compound having molecular weight equal to 2985.4 Da.

In one embodiment, a composition is provided comprising one or both of an isolated Ericin S compound having molecular weight equal to 3341.6 Da and an isolated Ericin A compound having molecular weight equal to 2985.4 Da in an amount suitable to confer protection against a pathogenic infection in a susceptible plant. The composition including one or both of the isolated Ericin S compound and the isolated Ericin A compound can further include at least one of the Fengycin-MA, -MB, -MC, -H, and -I compounds and the Dehydroxyfengycin-MA, -MB, and -I compounds and, optionally, also include one or a combination of additional isolated Fengycin- and Dehydroxyfengycin-like compounds.

The compositions including the Ericin S and Ericin A compounds and the at least one of the Fengycin-MA, -MB, -MC, -H, and -I compounds and the Dehydroxyfengycin-MA, -MB, and -I compounds and optionally the one or a combination of additional isolated Fengycin- and Dehydroxyfengycin-like compounds can further include one or a combination of a microbial, a biological, or a chemical insecticide, fungicide, nematicide, bacteriocide, herbicide, plant extract, plant growth regulator, or fertilizer, present in an amount suitable to benefit plant growth and/or to confer protection against pathogenic infection in the susceptible plant.

The fungicide can include an extract from Lupinus albus. The fungicide can include a BLAD polypeptide. The BLAD polypeptide can be a fragment of the naturally occurring seed storage protein from sweet lupine (Lupinus albus) that acts on susceptible fungal pathogens by causing damage to the fungal cell wall and disrupting the inner cell membrane. The fungicide can include about 20% of a BLAD polypeptide.

The compositions including the Ericin S and Ericin A compounds and the at least one of the Fengycin-MA, -MB, -MC, -H, and -I compounds and the Dehydroxyfengycin-MA, -MB, and -I compounds can be in the form of a liquid, an oil dispersion, a dust, a spreadable granule, or a dry wettable granule.

In one embodiment, a method is provided for benefiting plant growth and/or conferring protection against a plant pathogenic infection that includes applying an effective amount of the extract or the composition comprising the isolated Ericin S and Ericin A compounds and the Fengycin-MA, -MB, -MC, -H, and -I compounds and the Dehydroxyfengycin-MA, -MB, and -I compounds and one or a combination of additional isolated Fengycin-and Dehydroxyfengycin-like compounds to the plant or fruit, or to the roots or soil around the roots of the plants to benefit the plant growth and/or conferring protection against the plant pathogenic infection. The growth benefit of the plant and/or the conferred protection can be exhibited by improved seedling vigor, improved root development, improved plant growth, improved plant health, increased yield, improved appearance, improved resistance to plant pathogens, reduced pathogenic infection, or a combination thereof.

In the method for applying an effective amount of the extract or the composition comprising the isolated Ericin S and Ericin A compounds and the Fengycin-MA, -MB, -MC, -H, and -I compounds and the Dehydroxyfengycin-MA, -MB, - and -I compounds and one or a combination of additional isolated Fengycin-like or Dehydroxyfengycin-like compounds, the plant can include, for example, monocots, dicots, Cereals, Corn, Sweet Corn, Popcorn, Seed Corn, Silage Corn, Field Corn, Rice, Wheat, Barley, Sorghum, Asparagus, Berry, Blueberry, Blackberry, Raspberry, Loganberry, Huckleberry, Cranberry, Gooseberry, Elderberry, Currant, Caneberry, Bushberry, Brassica Vegetables, Broccoli, Cabbage, Cauliflower, Brussels Sprouts, Collards, Kale, Mustard Greens, Kohlrabi, Cucurbit Vegetables, Cucumber, Cantaloupe, Melon, Muskmelon, Squash, Watermelon, Pumpkin, Eggplant, Bulb Vegetables, Onion, Garlic, Shallots, Citrus, Orange, Grapefruit, Lemon, Tangerine, Tangelo, Pummelo, Fruiting Vegetables, Pepper, Tomato, Ground Cherry, Tomatillo, Okra, Grape, Herbs/Spices, Leafy Vegetables, Lettuce, Celery, Spinach, Parsley, Radicchio, Legumes/Vegetables (succulent and dried beans and peas), Beans, Green beans, Snap beans, Shell beans, Soybeans, Dry Beans, Garbanzo beans, Lima beans, Peas, Chick peas, Split peas, Lentils, Oil Seed Crops, Canola, Castor, Coconut, Cotton, Flax, Oil Palm, Olive, Peanut, Rapeseed, Safflower, Sesame, Sunflower, Soybean, Pome Fruit, Apple, Crabapple, Pear, Quince, Mayhaw, Root/Tuber and Corm Vegetables, Carrot, Potato, Sweet Potato, Cassave, Beets, Ginger, Horseradish, Radish, Ginseng, Turnip, Stone Fruit, Apricot, Cherry, Nectarine, Peach, Plum, Prune, Strawberry, Tree Nuts, Almond, Pistachio, Pecan, Walnut, Filberts, Chestnut, Cashew, Beechnut, Butternut, Macadamia, Kiwi, Banana, (Blue) Agave, Grass, Turf grass, Ornamental plants, Poinsettia, Hardwood cuttings, Chestnuts, Oak, Maple, sugarcane, or sugarbeet.

In the method for applying an effective amount of the extract or the composition comprising the isolated Ericin S and Ericin A compounds and the Fengycin-MA, -MB, -MC, -H, and -I compounds and the Dehydroxyfengycin-MA, -MB, - and -I compounds and one or a combination of additional isolated Fengycin-like or Dehydroxyfengycin-like compounds, the pathogenic infection can be caused by a plant pathogen, including, for example, a plant fungal pathogen, a plant bacterial pathogen, a rust fungus a Botrytis spp., a Botrytis cinerea, a Botrytis squamosa, an Erwinia spp., an Erwinia carotovora, an Erwinia amylovora, a Dickeya spp., a Dickeya dadantii, a Dickeya solani, an Agrobacterium spp., a Agrobacterium tumefaciens, a Xanthomonas spp., a Xanthomonas axonopodis, a Xanthomonas campestris pv. carotae, a Xanthomonas pruni, a Xanthomonas arboricola, a Xanthomonas oryzae pv. oryzae, a Xylella spp., a Xylella fastidiosa, a Candidatus spp., a Candidatus liberibacter, a Fusarium spp., a Fusarium colmorum, a Fusarium graminearum, a Fusarium oxysporum, a Fusarium oxysporum f. sp. Cubense, a Fusarium oxysporum f. sp. Lycopersici, a Fusarium virguliforme, a Sclerotinia spp., a Sclerotinia sclerotiorum, a Sclerotinia minor, Sclerotinia homeocarpa, a Cercospora/Cercosporidium spp., an Uncinula spp., an Uncinula necator (Powdery Mildew), a Podosphaera spp. (Powdery Mildew), a Podosphaera leucotricha, a Podosphaera clandestine, a Phomopsis spp., a Phomopsis viticola, an Alternaria spp., an Alternaria tenuissima, an Alternaria porri, an Alternaria alternate, an Alternaria solani, an Alternaria tenuis, a Pseudomonas spp., a Pseudomonas syringae pv. Tomato, a Phytophthora spp., a Phytophthora infestans, a Phytophthora parasitica, a Phytophthora sojae, a Phytophthora capsici, a Phytophthora cinnamon, a Phytophthora fragariae, a Phytophthora spp., a Phytophthora ramorum, a Phytophthora palmivara, a Phytophthora nicotianae, a Phakopsora spp., a Phakopsora pachyrhizi, a Phakopsora meibomiae an Aspergillus spp., an Aspergillus flavus, an Aspergillus niger, a Uromyces spp., a Uromyces appendiculatus, a Cladosporium spp., a Cladosporium herbarum, a Rhizopus spp., a Rhizopus arrhizus, a Penicillium spp., a Rhizoctonia spp., a Rhizoctonia solani, a Rhizoctonia zeae, a Rhizoctonia oryzae, a Rhizoctonia caritae, a Rhizoctonia cerealis, a Rhizoctonia crocorum, a Rhizoctonia fragariae, a Rhizoctonia ramicola, a Rhizoctonia rubi, a Rhizoctonia leguminicola, a Macrophomina phaseolina, a Magnaorthe oryzae, a Mycosphaerella spp., Mycosphaerella graminocola, a Mycosphaerella fijiensis (Black sigatoga), a Mycosphaerella pomi, a Mycosphaerella citri, a Magnaporthe spp., a Magnaporthe grisea, a Monilinia spp., a Monilinia fruticola, a Monilinia vacciniicorymbosi, a Monilinia laxa, a Colletotrichum spp., a Colletotrichum gloeosporiodes, a Colletotrichum acutatum, a Colletotrichum Candidum, a Diaporthe spp., a Diaporthe citri, a Corynespora spp., a Corynespora Cassiicola, a Gymnosporangium spp., a Gymnosporangium juniperi-virginianae, a Schizothyrium spp., a Schizothyrium pomi, a Gloeodes spp., a Gloeodes pomigena, a Botryosphaeria spp., a Botryosphaeria dothidea, a Neofabraea spp., a Wilsonomyces spp., a Wilsonomyces carpophilus, a Sphaerotheca spp., a Sphaerotheca macularis, a Sphaerotheca pannosa, a Erysiphe spp., a Stagonospora spp., a Stagonospora nodorum, a Pythium spp., a Pythium ultimum, a Pythium aphanidermatum, a Pythium irregularum, a Pythium ulosum, a Pythium lutriarium, a Pythium sylvatium, a Venturia spp, a Venturia inaequalis, a Verticillium spp., a Ustilago spp., a Ustilago nuda, a Ustilago maydis, a Ustilago scitaminea, a Claviceps spp., a Claviceps puprrea, a Tilletia spp., a Tilletia tritici, a Tilletia laevis, a Tilletia horrid, a Tilletia controversa, a Phoma spp., a Phoma glycinicola, a Phoma exigua, a Phoma lingam, a Cocliobolus sativus, a Gaeumanomyces gaminis, a Colleototricum spp., a Rhychosporium spp., Rhychosporium secalis, a Biopolaris spp., a Helminthosporium spp., a Helminthosporium secalis, a Helminthosporium maydis, a Helminthosporium solai, and a Helminthosporium tritici-repentis, or combinations thereof.

### EXAMPLES

The following Examples have been included to provide guidance to one of ordinary skill in the art for practicing representative embodiments of the presently disclosed subject matter. In light of the present invention and the general level of skill in the art, those of skill can appreciate that the following Examples are intended to be exemplary only and that numerous changes, modifications, and alterations can be employed without departing from the scope of the presently disclosed subject matter.

### Example 1

### Identification of a Bacterial Isolate as a Bacillus Amyloliquefaciens through Sequence Analysis

A plant associated bacterial strain, designated herein as RTI472, was isolated from the root of American ginseng grown in North Carolina. The16S rRNA and the rpoB genes of the RTI472 strain were sequenced and subsequently compared to other known bacterial strains in the NCBI and RDP databases using BLAST. It was determined that the 16S RNA partial sequence of RTI472 (SEQ ID NO: 1) is identical to the 16S rRNA gene sequence of Bacillus amyloliquefaciens strain NS6 (KF177175), Bacillus amyloliquefaciens strain FZB42 (NR_075005), and Bacillus subtilis subsp. subtilis strain DSM 10 (NR_027552). In addition, it was determined that the rpoB sequence of RTI472 has the highest level of sequence similarity to the known Bacillus amyloliquefaciens AS43.3 strain (i.e., 99% sequence identity); however, there is a 10 nucleotide difference on the DNA level, indicating that RTI472 is a new strain of Bacillus amyloliquefaciens. The RTI472 strain was identified as a Bacillus amyloliquefaciens. The differences in sequence for the rpoB gene at the DNA level indicate that RTI472 is a new strain of Bacillus amyloliquefaciens. The strain of Bacillus amyloliquefaciens RTI472 was deposited on 17 Apr. 2014 under the terms of the Budapest Treaty on the International Recognition of the Deposit of Microorganisms for the Purposes of Patent Procedure at the American Type Culture Collection (ATCC) in Manassas, Va., USA and bears the Patent Accession No. PTA-121166.

### Example 2

### Unique Genes Related to Lantibiotic Biosynthesis in Bacillus amyloliquefaciens RTI472 Strain

Further sequence analysis of the genome of the Bacillus amyloliquefaciens RTI472 strain revealed that this strain has unique genes related to lantibiotic biosynthesis, for which homologues are lacking in other closely related Bacillus amyloliquefaciens strains. The lantipeptide biosynthetic cluster was identified in Bacillus amyloliquefaciens RTI472 through computational analysis using the RAST, antiSMASH and BAGEL bioinformatics platforms. The RAST genome annotation identified a region with genes whose protein sequences were putatively involved in lantipeptide biosynthesis. This was confirmed by the antiSMASH data, which identified the same genomic region as harboring a lantipeptide biosynthetic cluster. FIG. 1 shows a schematic diagram of the genomic organization of the lantibiotic biosynthetic cluster found in Bacillus amyloliquefaciens RTI472 as compared to 2 closely related amyloliquefaciens strains. In FIG. 1, the top set of arrows represents protein coding regions for the RTI472 strain with relative direction of transcription indicated. For comparison, the corresponding regions for two closely related Bacillus amyloliquefaciens reference strains, FZB42 (middle) and TrigoCor1448 (bottom), are shown below the RTI472 strain. The degree of amino acid identity of the proteins encoded by the genes of the RTI472 strain as compared to the two reference strains is indicated both by the degree of shading of the representative arrows as well as a percentage identity indicated within the arrow. It can be observed from FIG. 1 that the FZB42 and TrigoCor1448 strains lack many of the genes present in this cluster, and there is a low degree of sequence identity within a number of the genes that are present.

Initial examination of the RAST data lead to the identification of the RTI472 strain genes belonging to a class I lantipeptide synthesis pathway, which seemed to be complete except for the prepeptide (the “A” gene of the cluster). Further examination of the RAST data for the RTI472 strain revealed a region (approximately 400 bp) between two genes (a putative biosynthetic gene and a putative immunity gene) that did not have any associated hypothetical genes. AntiSMASH was subsequently used to identify a putative core peptide sequence within this region. To confirm this result, the nucleotide sequence for this region was analyzed using the program BAGEL, which predicted a core peptide identified as Ericin S prepeptide. While the Bacillus amyloliquefaciens RTI472 appeared to lack the prepeptide for Ericin A based on the bioinformatic analysis, subsequent HPLC/MS/MS data revealed the presence of both Ericin A (MW of 2985.4 Da) and Ericin S (MW of 3341.6 Da) in the culture supernatant of the RTI472 strain. A comparison of metabolite profiles for the Bacillus amyloliquefaciens RTI472 strain and an unpublished Bacillus amyloliquefaciens strain designated “FB005” is shown in FIG. 2A. The graph in FIG. 2A shows that the RTI472 strain produces the Ericin A and Ericin S peptides while no Ericin A or Ericin S was detected for the FB005 strain.

Genome-genome comparisons between RTI472 and available Bacillus amyloliquefaciens genomes subsequently showed that none of the reference genomes harbor a functional Ericin S biosynthetic cluster. Many of the genomes do however have low homology hits to the resistance portion of this gene cluster. FIGS. 2B-2C are tables showing a comparison between the lantipeptide biosynthesis cluster identified in the RTI472 strain and 7 reference Bacillus amyloliquefaciens genomes. The tables show the loss of synteny in the region for the 7 reference Bacillus amyloliquefaciens genomes. None of the 7 reference strains harbor the functional Ericin S biosynthetic cluster. In addition, it was concluded based on a search of the literature that Bacillus subtilis A1/3 is the only other strain previously reported to have a biosynthetic cluster for production of Ericin S (see Stein et al., 2002, J. Bacteriol. Vol. 184, No. 6, 1703). Thus, the newly identified RTI472 strain possesses a lantipeptide synthesis pathway that produces an Ericin S molecule of MW=3341.6 Da and a Ericin A molecule of MW of 2985.4 Da, which is unique for a Bacillus amyloliquefaciens strain.

### Example 3

### Anti-Microbial Properties of Bacillus Amyloliquefaciens RTI472 Isolate

The antagonistic ability of the isolate against major plant pathogens was measured in plate assays. A plate assay for evaluation of antagonism against plant fungal pathogens was performed by growing the bacterial isolate and pathogenic fungi side by side on 869 agar plates at a distance of 4 cm. Plates were incubated at room temperature and checked regularly for up to two weeks for growth behaviors such as growth inhibition, niche occupation, or no effect. In the case of screening for antagonistic properties against bacterial pathogens, the pathogen was first spread as a lawn on 869 agar plates. Subsequently, 20 μl aliquots of a culture of RTI472 were spotted on the plate. Plates were incubated at room temperature and checked regularly for up to two weeks for an inhibition zone in the lawn around the positions were RTI472 had been applied. A summary of the antagonism activity is shown in Table I below.

TABLE I
Antagonistic properties of Bacillus amyloliquefaciens
RTI472 isolate against major plant pathogens

| Anti-Microbial Assays | RTI472 |
|---|---|
| Alternaria solani | +++ |
| Aspergillus flavus | ++ |
| Aspergillus nomius | +++ |
| Botrytis cinerea | +++ |
| Cercospora sojina | +++ |
| Fusarium colmorum | + |
| Fusarium graminearum | +++ |
| Fusarium oxysporum f. sp. Lycopersici | ++ |
| Fusarium oxysporum f. sp. cubense | ++ |
| Fusarium virguliforme | ++/+++ |
| Glomerella cingulata | +++ |
| Magnaporthe grisea | +++ |
| Monilina fructicola | +++ |
| Rhizoctonia solani | ++ |
| Sclerotinia homeocarpa | ++/+++ |
| Sclerotinia sclerotiorum | +++ |
| Septoria tritici | +− |
| Stagonospora nodorum | +++ |
| Phytophthora capsici | ++/+++ |
| Pythium sylvatium | + |
| Pythium aphanidermatum | ++ |
| Erwinia amylovora | + |
| Erwinia carotovora | + |
| Pseudomonas syringae pv. tomato | − |
| Ralstonia solenacearum | ++ |
| Xanthomonas euvesicatoria | ++ |

+++ very strong activity,
++ strong activity,
+ activity,
+− weak activity,
− no activity observed

### Example 4

### Phenotypic Traits of Bacillus amyloliquefaciens RTI472 Isolate

In addition to the antagonistic properties, various phenotypic traits were also measured for the Bacillus amyloliquefaciens 472 strain and the data are shown below in Table II. The assays were performed according to the procedures described in the text below Table II.

TABLE II
Phenotypic Assays: phytohormone production, acetoin
and indole acetic acid (IAA), and nutrient cycling of
Bacillus amyloliquefaciens RTI472 isolate.

| Characteristic Assays | RTI472 |
|---|---|
| Acid production (Methyl Red) | − |
| Acetoin production (MR-VP) | +++ |
| Chitinase activity | − |
| lndole-3-Acetic Acid production | − |
| Protease activity | ++ |
| Phosphate solubilization | + |

+++ very strong,
++ strong,
+ some,
+− weak,
− none observed

Acid and Acetoin Test. 20 μl of a starter culture in rich 869 media was transferred to 1 ml Methy Red—Voges Proskauer media (Sigma Aldrich 39484). Cultures were incubated for 2 days at 30 C 200 rpm. 0.5 ml culture was transferred and 50 μl 0.2 g/l methyl red was added. Red color indicated acid production. The remaining 0.5 ml culture was mixed with 0.3 ml 5% alpha-napthol (Sigma Aldrich N1000) followed by 0.1 ml 40% KOH. Samples were interpreted after 30 minutes of incubation. Development of a red color indicated acetoin production. For both acid and acetoin tests non-inoculated media was used as a negative control (Sokol et al., 1979, Journal of Clinical Microbiology. 9: 538-540).

Indole-3-Acetic Acid. 20 μl of a starter culture in rich 869 media was transferred to 1 ml 1/10 869 Media supplemented with 0.5 g/l tryptophan (Sigma Aldrich T0254). Cultures were incubated for 4-5 days in the dark at 30 C, 200 RPM. Samples were centrifuged and 0.1 ml supernatant was mixed with 0.2 ml Salkowski's Reagent (35% perchloric acid, 10 mM FeCl3). After incubating for 30 minutes in the dark, samples resulting in pink color were recorded positive for IAA synthesis. Dilutions of IAA (Sigma Aldrich 15148) were used as a positive comparison; non inoculated media was used as negative control (Taghavi, et al., 2009, Applied and Environmental Microbiology 75: 748-757).

Phosphate Solubilizing Test. Bacteria were plated on Pikovskaya (PVK) agar medium consisting of 10 g glucose, 5 g calcium triphosphate, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved. Zones of clearing were indicative of phosphate solubilizing bacteria (Sharma et al., 2011, Journal of Microbiology and Biotechnology Research 1: 90-95).

Chitinase activity. 10% wet weight colloidal chitin was added to modified PVK agar medium (10 g glucose, 0.2 g potassium chloride, 0.5 g ammonium sulfate, 0.2 g sodium chloride, 0.1 g magnesium sulfate heptahydrate, 0.5 g yeast extract, 2 mg manganese sulfate, 2 mg iron sulfate and 15 g agar per liter, pH7, autoclaved). Bacteria were plated on these chitin plates; zones of clearing indicated chitinase activity (N. K. S. Murthy & Bleakley., 2012. “Simplified Method of Preparing Colloidal Chitin Used for Screening of Chitinase Producing Microorganisms”. The Internet Journal of Microbiology. 10(2)).

Protease Activity. Bacteria were plated on 869 agar medium supplemented with 10% milk. Clearing zones indicated the ability to break down proteins suggesting protease activity (Sokol et al., 1979, Journal of Clinical Microbiology. 9: 538-540).

### Example EXAMPLE 5

### B. Amyloliquefaciens RTI472 Antagonism of Uromyces Appendiculatus and Botrytis Cinerea

Studies were performed in the greenhouse on common bean plants to determine the ability of the B. amyloliquefaciens RTI472 strain to prevent and/or ameliorate the effects of the plant pathogen bean rust (Uromyces appendiculatus) and the plant pathogen Botrytis cinerea.

In a first set of experiments, different formulations of the B. amyloliquefaciens RTI472 strain were tested for foliar application to control the plant pathogens Uromyces appendiculatus and Botrytis cinerea. In addition to the RTI472, a separate and newly identified B. amyloliquefaciens strain, RTI301, was also tested. The experimental design and formulations were as follows:

Formulations:

B. amyloliquefaciens RTI472 spores in Spent Fermentation Broth (SFB), diluted by a factor of about 100 in water, and applied to foliage at a rate of 1×10⁸ cfu/ml.

B. amyloliquefaciens RTI472 spores in Spent Fermentation Broth (SFB), diluted by a factor of about 100 in water with added yeast extract, and applied to foliage at a rate of 1×10⁸ cfu/ml and about 0.2% yeast extract.

B. amyloliquefaciens RTI301 spores in Spent Fermentation Broth (SFB), diluted by a factor of about 100 in water, and applied to foliage at a rate of 1×10⁸ cfu/ml.

B. amyloliquefaciens RTI301 spores in Spent Fermentation Broth (SFB), diluted by a factor of about 100 in water with added yeast extract, and applied to foliage at a rate of 1×10⁸ cfu/ml and about 0.2% yeast extract.

SERENADE OPTIMUM (BAYER CROP SCIENCE, INC) product was applied at a rate of B. subtilis strain QST713 spores at 1×10⁸ cfu/ml.

HORIZON (HORIZON AG-PRODUCTS) product was applied at a rate of 50 g a.i./ha (Tebuconazole).

BRAVO WEATHER STIK (SYNGENTA CROP PROTECTION, INC) product was applied at a rate of 500 g a.i./ha (Chlorothalonil).

TACTIC (LOVELAND PRODUCTS, INC) product was included in all the formulations listed above at the concentration of 0.1875% v/v.

Treatment Application Method:

A track sprayer was used to inoculate 21 day old common bean plants (having two trifoliates) with the various treatments listed above having a single overhead nozzle (TeeJet SS8001E Flat Fan) at a pressure=276 kPa (40 psi). The nozzle height was 36 cm (14″) above the bean plant leaves. The application volume was 200 L/ha and the number of repetitions in the experiment equaled six. The treatment plants were inoculated a single time along with control plants not receiving any treatment.

Infection Rate:

One day after treatment application, the test plants were infected with bean rust (Uromyces appendiculatus) at an inoculation rate of 200 k conidia/ml.

Nine days after infection with bean rust (Uromyces appendiculatus) the percent of disease control was evaluated for each of: RTI472 spores in Spent Fermentation Broth diluted with water alone (“RTI472+1% SFB”), RTI472 spores in Spent Fermentation Broth diluted with water plus yeast extract (“RTI472+1% SFB+Yeast Extract”), RTI301 spores in Spent Fermentation Broth diluted 100 fold with water alone (“RTI301+1% SFB”), RTI301 spores in Spent Fermentation Broth diluted 100 fold with water plus yeast extract (“RTI301+1% SFB+Yeast Extract”), BRAVO WEATHER STIK, HORIZON, and SERENADE OPTIMUM according to the rates of application described above. The non-treated control (water only) resulted in 28% disease.

The results of the experiment are shown in Table III below. The results indicate that the addition of the yeast extract for each of the RTI472 and RTI301 strains resulted in about a 40% increase in disease control as compared to the strains applied without the addition of yeast extract. The amount of disease control exhibited by RTI472+1% SFB+Yeast Extract and RTI301+1% SFB+Yeast Extract was similar to that observed for SERENADE OPTIMUM when applied at the same rate (i.e., 1×10⁸ cfu/ml) even though the amount of SFB in the RTI472 and RTI301 formulations was relatively low at 1%, and the SFB can be expected to contain secreted compounds having antifungal activity.

Similar results were observed for an experiment performed in pepper plants using the same formulations and experimental design listed above to measure the amount of disease control exhibited by RTI472 and RTI301 for Pepper Botrytis Blight caused by the pathogen Botrytis cinerea (data not shown).

TABLE III
Results of B. amyloliquefaciens RTI472 and RTI301
control of Bean Rust (Uromyces appendiculatus) as
compared to SERENADE OPTIMUM and chemical active agents
when formulated with and without yeast extract.

| | Percent |
|---|---|
| Treatment | Disease Control |
| RTI472 + 1% SFB (1 × 10⁸ cfu/ml) | 53 ab |
| RTI472 + 1% SFB + Yeast Extract | 96 cd |
| (1 × 10⁸ cfu/ml) | |
| RTI301 + 1% SFB (1 × 10⁸ cfu/ml) | 57 ab |
| RTI301 + 1% SFB + Yeast Extract | 96 cd |
| (1 × 10⁸ cfu/ml) | |
| BRAVO WEATHER STIK | 100 d |
| HORIZON | 100 d |
| SERENADE OPTIMUM (1 × 10⁸ cfu/ml) | 92 cd |
| Percent disease in non-treated control plants | |
| was 28% | |

The following experiment describes disease control of bean rust by RTI472 caused by the plant pathogen Uromyces appendiculatus. The experimental design and formulations were as follows:

Formulations:

B. amyloliquefaciens RTI472 spores were in Spent Fermentation Broth (SFB, diluted by a factor of about 100 in water with added yeast extract, and applied to foliage at a rate of 1×10⁸ cfu/ml and about 0.2% yeast extract.

SERENADE OPTIMUM (BAYER CROP SCIENCE, INC) product was applied at a rate of spores at 1×10⁸ cfu/ml and 4×10⁸ cfu/ml.

HORIZON (HORIZON AG-PRODUCTS) was applied at a rate of 50 g a.i./ha (Tebuconazole).

Chlorothalonil was applied at a rate of 500 g a.i./ha.

TACTIC (LOVELAND PRODUCTS, INC), included as a blank control, was also applied at a concentration of 0.1875% v/v to all formulations.

Treatment Application Method:

A track sprayer was used to inoculate 21 day old common bean plants (having two trifoliates) with the various treatments having a single overhead nozzle (TeeJet SS8001E Flat Fan) at a pressure=276 kPa (40 psi). The nozzle height was 36 cm (14″) above the soybean plant leaves. The application volume was 200 L/ha and the number of repetitions in the experiment equaled six. The treatment plants were inoculated a single time along with control plants not receiving any treatment.

Infection Rate:

One day after treatment application, the test plants were infected with bean rust (Uromyces appendiculatus) at an inoculation rate of 200 k conidia/ml.

Ten days after infection with bean rust (Uromyces appendiculatus) the percent of disease control was evaluated for each of: the RTI472 spores in Spent Fermentation Broth (SFB) (applied at 1×10⁸ cfu/ml), Tebuconazole (applied at 50 g a.i./ha), Chlorothalonil (applied at 500 g a.i./ha), SERENADE OPTIMUM (applied at 1×10⁸ cfu/ml), and SERENADE OPTIMUM (applied at 4×10⁸ cfu/ml). TACTIC (applied at 0.1875%), also included as a control, was applied to all formulations. The non-treated controls resulted in 23% disease (data not shown). The results of the experiment are shown in Table IV below and in FIG. 3, and indicate a similar level of control of Bean Rust (Uromyces appendiculatus) as compared to SERENADE OPTIMUM when applied at the same rate.

TABLE IV
Results of B. amyloliquefaciens RTI472 control
of Bean Rust (Uromyces appendiculatus) as compared
to SERENADE OPTIMUM and chemical active agents.

| | Percent |
|---|---|
| Treatment | Disease Control |
| RTI472 + SFB 1 × 10⁸ cfu/ml | 92 a |
| 0.1875% TACTIC | 44 b |
| Tebuconazole | 100 a |
| Chlorothalonil | 100 a |
| SERENADE OPTIMUM 1 × 10⁸ cfu/ml | 95 a |
| SERENADE OPTIMUM 4 × 10⁸ cfu/ml | 97 a |
| Percent disease in non-treated control | |
| plants - 23% | |

### Example 6

### B. amyloliquefaciens RTI472 Antagonism on Soybean Powdery Mildew (Microsphaera diffusa)

Studies were performed in the greenhouse on soybean to determine the ability of the B. amyloliquefaciens RTI472 strain to prevent and/or ameliorate the effects of natural infestation of the soybean powdery mildew (Microsphaera diffusa).

Formulations:

B. amyloliquefaciens RTI472 spores were in Spent Fermentation Broth (SFB), diluted by a factor of about 100 in water with added yeast extract, and applied to foliage at a rate of 1×10⁸ cfu/ml and about 0.2% yeast extract.

SERENADE OPTIMUM (BAYER CROP SCIENCE, INC) spores were applied at a rate of 1×10⁸ cfu/ml.

HORIZON (HORIZON AG-PRODUCTS) was applied at a rate of 50 g a.i./ha (Tebuconazole).

TACTIC (LOVELAND PRODUCTS, INC) was applied at a concentration of 0.1875% v/v to all formulations.

Treatment Application Method:

A track sprayer was used to inoculate 21 day old soybean plants (having two trifoliates) with the various treatments having a single overhead nozzle (TeeJet SS8001E Flat Fan) at a pressure=276 kPa (40 psi). The nozzle height was 36 cm (14″) above the soybean plant leaves. The application volume was 200 L/ha and the number of repetitions in the experiment equaled 6. The plants receiving treatment were inoculated a single time along with control plants not receiving any treatment.

Infection Rate:

Immediately after treatment application, the test plants were placed between flats of soybeans previously infected with Microsphaera diffusa.

Nine days after being placed between the Microsphaera diffusa infected flats, the percent of disease dontrol was evaluated for each of: the RTI472 spores in Spent Fermentation Broth (SFB) (applied at 1×10⁸ cfu/ml), SERENADE OPTIMUM (applied at 1×10⁸ cfu/ml), and HORIZON (Tebuconazole applied at a 50 g a.i./ha). The non-treated controls resulted in 70% disease (data not shown). The results of the experiment are shown in Table V below and in FIG. 4, and indicate a superior control of Soybean Powdery Mildew (Microsphaera diffusa) as compared to SERENADE OPTIMUM when applied at the same rate.

TABLE V
Results of B. amyloliquefaciens RTI472 control
of Soybean Powdery Mildew (Microsphaera diffusa)
as compared to SERENADE OPTIMUM.

| | Percent |
|---|---|
| Treatment | Disease Control |
| RTI472 + SFB 1 × 10⁸ cfu/ml | 76 ab |
| SERENADE OPTIMUM 1 × 10⁸ cfu/ml | 56 bc |
| Tebuconazole | 100 a |
| Percent disease in non-treated control | |
| plants - 70% | |

The graph in FIG. 4 shows the % disease control (mean) on the y axis 9 days after inoculation with treatment for each of: the RTI472 spores in Spent Fermentation Broth (SFB) (applied at 1×10⁸ cfu/ml), SERENADE OPTIMUM (applied at 1×10⁸ cfu/ml), and HORIZON (Tebuconazole applied at a 50 g a.i./ha). The check controls resulted in 70% disease (data not shown).

### Example 7

### B. Amyloliquefaciens RTI472 Antagonism on Pepper Botrytis Blight (Botrytis cinerea)

Studies were performed in the greenhouse on pepper to determine the ability of the B. amyloliquefaciens RTI472 strain to prevent and/or ameliorate the effects of the plant pathogen Pepper Botrytis Blight (Botrytis cinerea).

Formulations:

B. amyloliquefaciens RTI472 spores were in Spent Fermentation Broth (SFB), diluted by a factor of about 100 in water with added yeast extract, and applied to foliage at a rate of 1×10⁸ cfu/ml and about 0.2% yeast extract.

SERENADE OPTIMUM (BAYER CROP SCIENCE, INC) spores were applied at a rate of 1×10⁸ cfu/ml and 4×10⁸ cfu/ml.

HORIZON (HORIZON AG-PRODUCTS) was applied at a rate of 50 g a.i./ha (Tebuconazole).

Chlorothalonil was applied at a rate of 500 g a.i./ha.

TACTIC (LOVELAND PRODUCTS, INC), included as a blank control, was also applied at a concentration of 0.1875% v/v to all formulations.

Treatment Application Method:

A track sprayer was used to inoculate 28 day old pepper plants with the various treatments having a single overhead nozzle (TeeJet SS8001E Flat Fan) at a pressure=276 kPa (40 psi). The nozzle height was 36 cm (14″) above the pepper plant leaves. The application volume was 200 L/ha and the number of repetitions in the experiment equaled six. The treatment plants were inoculated a single time along with control plants not receiving any treatment.

Infection Rate:

One day following treatment application, the test plants were infected with Botrytis cinerea at an infection rate of 1M conidia/ml.

Seven days following infection with Pepper Botrytis Blight (Botrytis cinerea) the percent of disease dontrol was evaluated for each of: the RTI472 spores in Spent Fermentation Broth (SFB) (applied at 1×10⁸ cfu/ml), TACTIC (applied at 0.1875%), Tebuconazole (applied at 50 g a.i./ha) (Chlorothalonil applied at 500 g a.i./ha), SERENADE OPTIMUM (applied at 1×10⁸ cfu/ml), and SERENADE OPTIMUM (applied at 4×10⁸ cfu/ml). The non-treated controls resulted in 6% disease (data not shown). The results of the experiment are shown in Table VI below and in FIG. 5, and indicate a superior control of Pepper Botrytis Blight (Botrytis cinerea) as compared to SERENADE OPTIMUM when applied at the same rate.

TABLE VI
Results of B. amyloliquefaciens RTI472 control of
Pepper Botrytis Blight (Botrytis cinerea) as compared
to Serenade Optimum and chemical active agents.

| | Percent |
|---|---|
| Treatment | Disease Control |
| RTI472 + SFB 1 × 10⁸ cfu/ml | 88 ab |
| 0.1875% TACTIC | 0 c |
| Tebuconazole | 99 a |
| Chlorothalonil | 99 a |
| SERENADE OPTIMUM 1 × 10\hat{ }8 cfu/ml | 77 b |
| SERENADE OPTIMUM 4 × 10\hat{ }8 cfu/ml | 88 ab |
| Percent disease in non-treated control | |
| plants - 6% | |

In another experiment, studies were performed in the greenhouse on pepper to determine the optimal dose of the B. amyloliquefaciens RTI472 strain to prevent and/or ameliorate the effects of the plant pathogen Pepper Botrytis Blight (Botrytis cinerea).

Formulations:

B. amyloliquefaciens RTI472 spores were in Spent Fermentation Broth (SFB) with added yeast extract and applied to foliage at an application rate ranging from 2.5×10⁶ cfu/ml to 2.5×10⁸ cfu/ml and from 0.01% to 0.2% yeast extract.

SERENADE OPTIMUM (BAYER CROP SCIENCE, INC) spores were used at application rates ranging from 2.5×10⁶ cfu/ml to 4×10⁸ cfu/ml.

HORIZON (HORIZON AG-PRODUCTS) was applied at a rate of 50 g a.i./ha (Tebuconazole)

TACTIC (LOVELAND PRODUCTS, INC), also included as a blank control, was applied at a concentration of 0.1875% v/v to all formulations.

Treatment Application Method:

A track sprayer was used to inoculate 28 day old pepper plants with the various treatments having a single overhead nozzle (TeeJet SS8001E Flat Fan) at a pressure=276 kPa (40 psi). The nozzle height was 36 cm (14″) above the pepper plant leaves. The application volume was 200 L/ha and the number of repetitions in the experiment equaled six. The treatment plants were inoculated a single time along with control plants not receiving any treatement.

Infection Rate:

One day following treatment application, the test plants were infected with Botrytis cinerea at an inoculation rate of 1M conidia/ml.

Three days after infection with Pepper Botrytis Blight (Botrytis cinerea) the percent of disease control was evaluated for each of: the RTI472 spores in Spent Fermentation Broth (SFB) (applied at 2.5×10⁶, 1×10⁷, 2.5×10⁷, 1×10⁸, and 2.5×10⁸ cfu/m1) and SERENADE OPTIMUM (applied at 2.5×10⁶, 1×10⁷, 2.5×10⁷, 1×10⁸, and 2.5×10⁸ cfu/m1) as compared to TACTIC (applied at 0.1875% to all formulations as well as used as a control), SERENADE OPTIMUM (applied at 4×10⁸), and Tebuconazole (applied at 50 g a.i./ha). The non-treated controls resulted in 70% disease (data not shown). The results of the experiment are shown in Table VII below and in FIG. 6.

TABLE VII
Results of B. amyloliquefaciens RTI472 dose response experiment
for control of Pepper Botrytis Blight (Botrytis cinerea) as
compared to SERENADE OPTIMUM and chemical active agents.

| | Percent Disease Control | | | | |
|---|---|---|---|---|---|
| Rate: | | | | Serenade | |
| CFU/ | RTI472 + | Serenade | 0.1875% | Optimum | Tebucon- |
| ml | SFB | Optimum | Tactic | 4 × 10⁸ | azole |
| 2.5 × 10⁶ | 36 efg | 22 fgh | n/a | n/a | n/a |
| 1.0 × 10⁷ | 51 cdef | 45 defg | n/a | n/a | n/a |
| 2.5 × 10⁷ | 62 bcde | 72 abcd | n/a | n/a | n/a |
| 1.0 × 10⁸ | 96 a | 93 ab | n/a | n/a | n/a |
| 2.5 × 10⁸ | 98 a | n/a | n/a | n/a | n/a |

| | | | |
|---|---|---|---|
| Percent disease in non-treated | 14 gh | 95 ab | 100 a |
| control plants- 30% | | | |

In another experiment, studies were performed in the greenhouse on pepper to determine the ability of the B. amyloliquefaciens RTI472 strain to prevent and/or ameliorate the effects of the plant pathogen Pepper Botrytis Blight (Botrytis cinerea) at varying rates of inoculation of the pathogen.

Formulations:

B. amyloliquefaciens RTI472 spores were in Spent Fermentation Broth (SFB) diluted by a factor of about 100 in water with added yeast extract, and applied to foliage at a rate of 1×10⁸ cfu/ml and about 0.2% yeast extract.

SERENADE OPTIMUM (BAYER CROP SCIENCE, INC) spores were used at application rates of 1×10⁸ cfu/ml to 4×10⁸ cfu/ml.

HORIZON (HORIZON AG-PRODUCTS) was applied at a rate of 50 g a.i./ha (Tebuconazole) TACTIC (LOVELAND PRODUCTS, INC), also included as a blank control, was applied at a concentration of 0.1875% v/v to all formulations.

Treatment Application Method:

A track sprayer was used to inoculate 28 day old pepper plants with the various treatments having a single overhead nozzle (TeeJet SS8001E Flat Fan) at a pressure=276 kPa (40 psi). The nozzle height was 36 cm (14″) above the pepper plant leaves. The application volume was 200 L/ha and the number of repetitions in he experiment equaled six. The treatment plants were inoculated a single time along with control plants not receiving any treatement.

Infection Rate:

One day following treatment application, the test plants were infected with Botrytis cinerea at an inoculation rate ranging from 50 k to 2M conidia/ml.

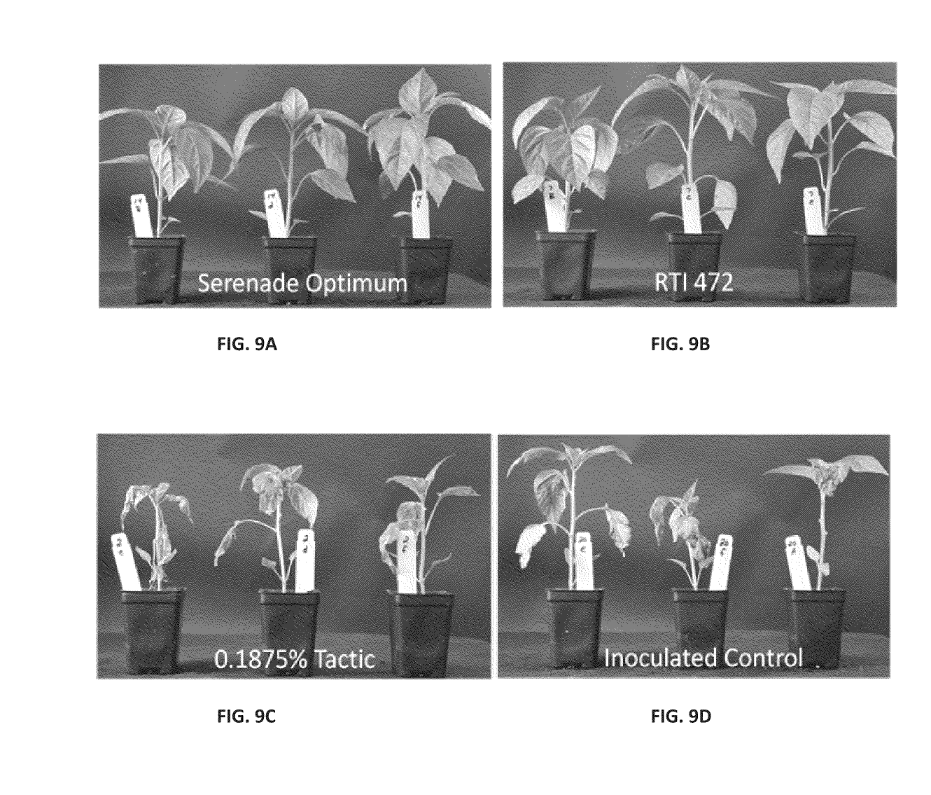

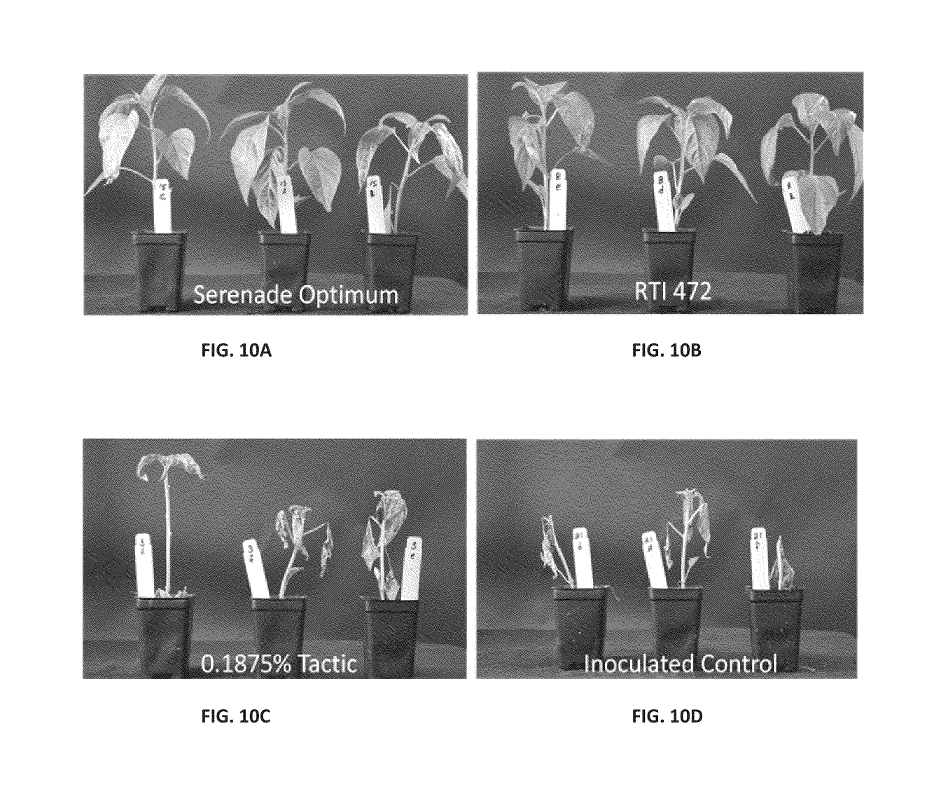

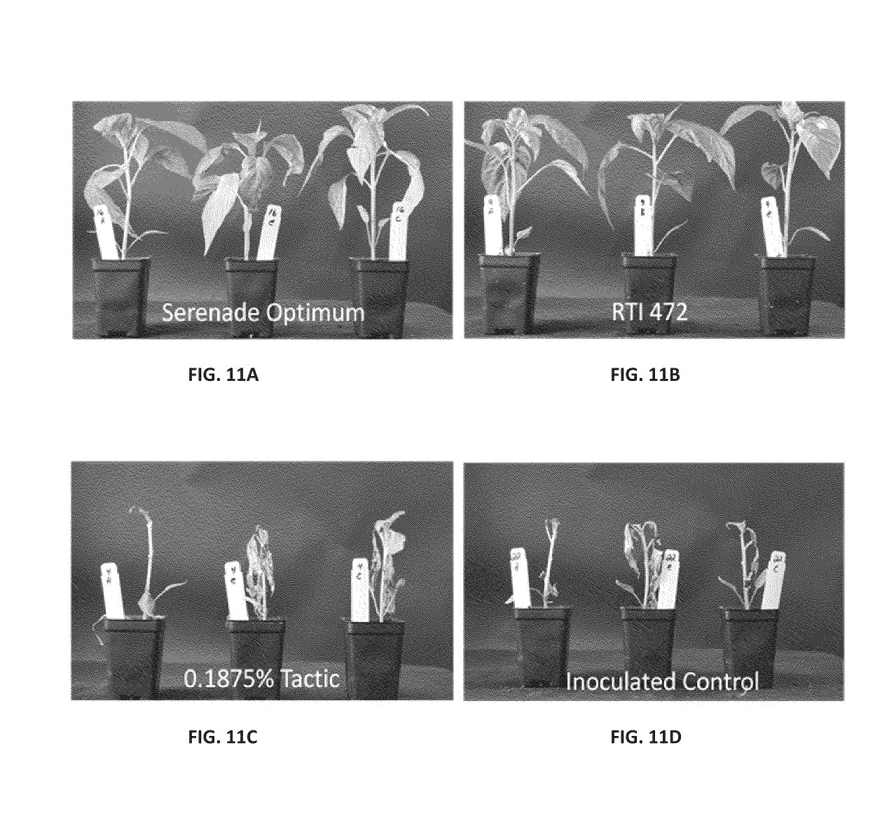

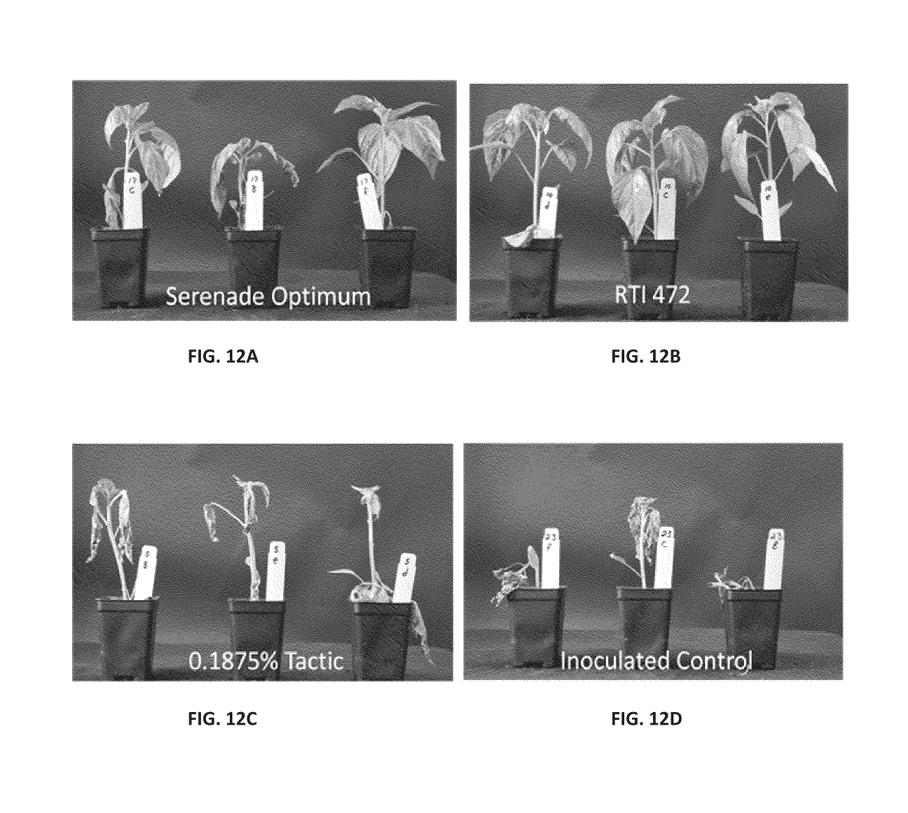

Four days after infection with Pepper Botrytis Blight (Botrytis cinerea) the percent of disease control was evaluated for each of: RTI472 spores in Spent Fermentation Broth (SFB) (applied at 2.5×10⁶ cfu/ml) and SERENADE OPTIMUM (applied at 1×10⁸ and 4×10⁸ cfu/ml) as compared to TACTIC (applied at 0.1875% to all formulations as well as used as a control) and Tebuconazole (HORIZON; applied at 50 g a.i./ha). The percent disease in the non-treated controls (data not shown) was 50 k=15%, 100 k=30%, 500 k=65%, 1M=65%, and 2M=65%. The results of the experiment are shown in Table VIII below and in FIG. 7. In addition, FIGS. 8A-8D show images of the plants at the 50 k inoculation rate with Botrytis cinerea, FIGS. 9A-9D show images of the plants at the 100 k inoculation rate with Botrytis cinerea, FIGS. 10A-10D show images of the plants at the 500 k inoculation rate with Botrytis cinerea, FIGS. 11A-11D show images of the plants at the 1M inoculation rate with Botrytis cinerea, and FIGS. 12A-12D show images of the plants at the 2M inoculation rate with Botrytis cinerea. At the 3 lowest pathogen inoculation rates, a similar percentage of disease control was observed for the RTI472 strain and SERENADE OPTIMUM. However, a statistical improvement was observed for the plants treated with the RTI472 strain as compared to SERENADE OPTIMUM at the 2 highest rates of pathogen inoculation, 1M and 2M.

TABLE VIII
Results of B. amyloliquefaciens RTI472 ability to control of Pepper Botrytis Blight
(Botrytis cinerea) after inoculation at varying concentration of the plant pathogen
as compared to SERENADE OPTIMUM and chemical active agents.

| | | Percent Disease Control | | | | |
|---|---|---|---|---|---|---|
| | | RTI472 + | Serenade | | Serenade | |
| Rate: | Percent | SFB | Optimum | 0.1875% | Optimum | |
| Conidia/ml | Disease | 1 × 10⁸ | 1 × 10⁸ | Tactic | 4 × 10⁸ | Tebuconazole |
| 50K | 15 | 99 a | 98 a | 0 d | n/a | n/a |
| 100K | 30 | 100 a | 99 a | 0 d | n/a | n/a |
| 500K | 65 | 93 a | 96 a | 0 d | n/a | n/a |
| 1M | 65 | 89 a | 83 abc | 0 d | 88 ab | 100 a |
| 2M | 65 | 55 c | 59 bc | 0 d | n/a | 94 a |

### Example 8

### B. Amyloliquefaciens RTI472 Antagonism of Powdery Mildew in Cucurbits

Studies were performed in field trials of cucurbits in Florida to determine the ability of the B. amyloliquefaciens RTI472 strain to prevent and/or ameliorate the effects of the plant pathogen Cucurbit Disease Powdery Mildew.

In total, 6 applications were made for each product using the rates as indicated in Table IX below. In the case of RTI472, the application rate was in g/ha and the application rate corresponded to the same colony forming units/ha as recommended for SERENADE OPTIMUM based on a 1400 g/ha application rate.

All applications were performed 6 times with 5 to 7 day intervals between applications unless otherwise specified. The timing of the first application depended on the particular crop and ranged from at the time of planting, a fews weeks after crop emergence, at the time of flowering, upon disease emergence, or just prior to expectation of disease emergence. In the case of the programs 4, 5 and 6, which are combining the application of a biological with a chemical active ingredient, the first, third and fifth applications (A, C and E) were made with the biological, while the second, fourth and sixth application (B, D and F) were made with the chemical.

Formulations:

SERENADE OPTIMUM (BAYER CROP SCIENCE, INC) was applied at a rate of 1400 g/ha, corresponding to 1.8×10⁺¹³ CFU/ha.

B. amyloliquefaciens RTI472 spores were in Spent Fermentation Broth (SFB) with added yeast extract and the application rate corresponded to the same colony forming units/ha (CFU/ha) as recommended for SERENADE OPTIMUM based on a 1400 g/ha application rate and yeast extract ranging from about 0.01% to 0.2%.

TACTIC (LOVELAND PRODUCTS, INC) was applied at a concentration of 0.1875% v/v.

LUNA EXPERIENCE (BAYER CROP SCIENCE, INC) was applied at a rate of 500 ga.i./ha (Fluopyram plus Tebuconazole fungicide).

BRAVO WEATHER STIK (SYNGENTA CROP PROTECTION, INC) was applied at a rate of 2240 ga.i./ha (Chlorothalonil).

The experimental design was as follows: Untreated control, RTI472+TACTIC, SERENADE OPTIMUM+TACTIC, RTI472+LUNA EXPERIENCE+TACTIC, SERENADE OPTIMUM+LUNA EXPERIENCE+TACTIC, and BRAVO WEATHER STIK+LUNA EXPERIENCE+TACTIC.

Treatment Application Method:

Six applications were made with timing as described above. The application sprayer was set up to deliver 30 gallons per acre. The individual plots were sprayed at a ground speed of 4 mph using a CO₂ backpack sprayer with flat fan nozzles (8004 type) and each nozzle was spaced 18 inches apart. The carrier to deliver the chemical was water mixed in a 2 liter bottle.

Disease Scoring: The mean percent of disease serverity was evaluated for 5 leaves of a plant for each of the treatments. Four plots from each treatment were evaluated for crop reponse and disease control of the powdery mildew. The trials were rated after each application just prior to the next application. The powdery mildew was from a natural infestation. The results of the experiment for cucumber are shown in Table IX below and indicate a similar control of powdery mildew in cucumbers as compared to SERENADE OPTIMUM when applied at the same rate as a stand alone biofungicide or as part of a treatment program.

TABLE IX
Results of B. amyloliquefaciens RTI472 control
of Powdery mildew in Cucumbers as compared to SERENADE
OPTIMUM and other chemical active agents.

| | | Mean % |
|---|---|---|
| | | disease |
| | Powdery mildew in cucumbers | Severity |
| 1 | Untreated control | 50 a |
| 2 | RTI472 + TACTIC | 25 b |
| 3 | SERENADE OPTIMUM + TACTIC | 30 b |
| 4 | RTI472 + LUNA EXPERIENCE + TACTIC | 10 c |
| 5 | SERENADE OPTIMUM + LUNA EXPERIENCE + | 3 c |
| | TACTIC | |
| 6 | BRAVO WEATHER STIK + LUNA EXPERIENCE + | 10 c |
| | TACTIC | |

Similar experiments were performed using the same format and application rates in commercial production greenhouse field trials in butternut squash and cucumber to determine the ability of the RTI472 strain to prevent and/or ameliorate the effects of the plant pathogen powdery mildew. For cucumber and butternut squash, the first treatment application occurred about 3 weeks after crop emergence. The data are shown below for butternut squash and cucumber in Tables X and XI, respectively. Disease severity, expressed as “Area Under Disease Pressure Curve” (AUDPC), was determined in function of severity (% leaf area affected) of powdery mildew on the upper leaf surface.

Compared to SERENADE OPTIMUM, RTI472 provided better control as a stand-alone biofungicide against powdery mildew on butternut squash. The best control of powdery mildew on butternut squash was observed for the three programs, where the combination of B. amyloliquefaciens RTI472 and LUNA EXPERIENCE (BAYER CROP SCIENCE) was equal to the industry standard program based on the use of BRAVO WEATHER STIK (SYNGENTA) combined with LUNA EXPERIENCE. Therefore, B. amyloliquefaciens RTI472 could be used as an alternative to the Chlorothalonil fungicide in a program for the control powdery mildew on cucurbits. This was further confirmed in trials to control powdery mildew on cucumber, in which B. amyloliquefaciens RTI472 as a stand-alone showed similar performance as the programs using the industry standard LUNA EXPERIENCE (see Table XI).

TABLE X
Results of B. amyloliquefaciens RTI472 control of
Powdery mildew in butternut squash as compared to SERENADE
OPTIMUM and other chemical active agents.

| | | | | | AUDPC |
|---|---|---|---|---|---|
| | | | Rate in | | Severity - |
| | | Descrip- | g/ha* or | | upper leaf |
| No. | Name | tion | gai/ha | Code⁺ | surface |
| 1 | UNTREATED | | | | 83 |
| 2 | RTI472 | Biological | 705* | ABCDEF | 33 |
| 3 | SERENADE | Bio. Std. | 1400* | ABCDEF | 61 |
| | OPTIMUM | | | | |
| 4 | RTI472 | Biological | 705* | ACE | 12 |
| | LUNA | Chem. Std. | 500 | BDF | |
| | EXPERIENCE | | | | |
| 5 | SERENADE | Bio. Std. | 1400* | ACE | 9 |
| | OPTIMUM | | | | |
| | LUNA | Chem. Std. | 500 | BDF | |
| | EXPERIENCE | | | | |
| 6 | BRAVO | Chem. Std. | 2240 | ACE | 12 |
| | WEATHER | | | | |
| | STIK | | | | |
| | LUNA | Chem. Std. | 500 | BDF | |
| | EXPERIENCE | | | | |

*application rate in g/ha
⁺Indicates timing of applications (have letter/date)

TABLE XI
Results of B. amyloliquefaciens RTI472 control
of Powdery mildew in cucumber as compared to SERENADE
OPTIMUM and other chemical active agents.

| | | | | | AUDPC |
|---|---|---|---|---|---|
| | | | Rate in | | Severity - |
| | | Descrip- | g/ha* or | | upper leaf |
| No. | Name | tion | gai/ha | Code⁺ | surface |
| 1 | UNTREATED | | | | 896 |
| 2 | RTI472 | Biological | 705* | ABCDEF | 327 |
| 3 | SERENADE | Bio. Std. | 1400* | ABCDEF | 405 |
| | OPTIMUM | | | | |
| 4 | RTI472 | Biological | 705* | ACE | 390 |
| | LUNA | Chem. Std. | 500 | BDF | |
| | EXPERIENCE | | | | |
| 5 | SERENADE | Bio. Std. | 1400* | ACE | 359 |
| | OPTIMUM | | | | |
| | LUNA | Chem. Std. | 500 | BDF | |
| | EXPERIENCE | | | | |
| 6 | BRAVO | Chem. Std. | 2240 | ACE | 358 |
| | WEATHER | | | | |
| | STIK | | | | |
| | LUNA | Chem. Std. | 500 | BDF | |
| | EXPERIENCE | | | | |

*application rate in g/ha
⁺Indicates timing of applications (have letter/date)

### Example 9

### B. Amyloliquefaciens RTI472 Antagonism of White Mold and Leaf Spot in Snap Bean and Peanut

Studies were performed in field trials of snap bean and peanut to determine the ability of the B. amyloliquefaciens RTI472 strain to prevent and/or ameliorate the effects of the plant pathogens cottony rot (white mold) caused by Sclerotinia sclerotiorum in snap beans and leaf spot in peanut.

In total, 6 applications were made with 5 to 7 day intervals between applications for each product using the rates as indicated in Table XII below unless otherwise specified. The timing of the first application depended on the particular crop and ranged from at the time of planting, a fews weeks after crop emergence, at the time of flowering, upon disease emergence, or just prior to expectation of disease emergence. In the case of RTI472 (* application rate in g/ha), the application rate corresponded to the same colony forming units/ha as recommended for SERENADE OPTIMUM based on a 1400 g/ha application rate. In the case of the programs 4, 5 and 6, which are combining the application of a biological with a chemical active ingredient, the first, third and fifth applications (A, C and E) were made with the biological, while the second, fourth and sixth application (B, D and F) were made with the chemical.

Formulations:

SERENADE OPTIMUM (BAYER CROP SCIENCE, INC) was applied at a rate of 1400 g/ha, corresponding to 1.8×10 ⁺¹³ CFU/ha.

B. amyloliquefaciens RTI472 spores were in Spent Fermentation Broth (SFB) with added yeast extract and the application rate corresponded to the same colony forming units/ha as recommended for SERENADE OPTIMUM based on a 1400 g/ha application rate with yeast extract ranging from about 0.01% to 0.2%.

TACTIC (LOVELAND PRODUCTS, INC) was applied at a concentration of 0.1875% v/v.

TOPSIN M 70W (CEREXAGRI, INC.) was applied at a rate of 1570 ga.i./ha (Thiophanate-methyl fungicide).

PROLINE (BAYER CROP SCIENCE) was applied at a rate of 200 ga.i./ha (Prothioconazole fungicide).

The experimental design was as follows: Untreated control, RTI472+TACTIC, SERENADE OPTIMUM+TACTIC, RTI472+TOPSIN M 70W+TACTIC, SERENADE OPTIMUM+TOPSIN M 70W+TACTIC, and PROLINE+TOPSIN M 70W+TACTIC.

Treatment Application Method: All applications were performed 6 times with 5 to 7 day intervals between applications unless otherwise specified. The application sprayer was setup to deliver 30 gallons per acre. The individual plots were sprayed at a ground speed of 4 mph using a CO₂ backpack sprayer with flat fan nozzles (8004 type) and each nozzle was spaced 18 inches apart. The carrier to deliver the chemical was water mixed in a 2 liter bottle.

Disease Scoring:

White Mold Trial in snap bean: Stem evaluation—Ten stems per plot were evaluated. The disease severity was determined by visual estimation of the amount of each stem that was affected by the disease. Canopy—The leaves, developing and mature fruit were evaluated on the plant. Six, 2 foot sections per plot were evaluated as subsamples and the severity was determined by estimating the percentage of the canopy that showed symptoms of white mold. The entire canopy was evaluated.

The results of the experiment are shown in Table XII below. Compared to SERENADE OPTIMUM, RTI472 provided better control as a stand-alone biofungicide against white mold on snap beans. The best control of white mold on snap beans was observed for the program using the combination of B. amyloliquefaciens RTI472 and TOPSIN (Thiophanate-methyl fungicide), which was even better than the chemical program based on the use of PROLINE (Prothioconazole fungicide) combined with TOPSIN. Therefore, B. amyloliquefaciens RTI472 could be used in programs as an alternative to Prothioconazole fungicide for the control of diseases such as Cercospora leaf spot, Septoria brown spot, Sclerotinia and Rhizoctonia that are found on Dry beans, Canola, Corn, Peanuts, Soybean and Sugarbeets.

TABLE XII
Results of B. amyloliquefaciens RTI472 control of white mold in snap bean
as compared to SERENADE OPTIMUM and other chemical active agents.

| | | | | | | AUDPC |
|---|---|---|---|---|---|---|
| | | | | | AUDPC | Canopy |
| | | | | | Stem Mold | Mold |
| No. | Name | Description | Rate g/ha | Code⁺ | severity | severity |
| 1 | UNTREATED | | | | 457 | 621 |
| 2 | RTI472 | Biological | 705* | ABCDEF | 237 | 450 |
| 3 | SERENADE | Bio. Std. | 1400* | ABCDEF | 274 | 499 |
| | OPTIMUM | | | | | |
| 4 | RTI472 | Biological | 705* | ACE | 121 | 150 |
| | TOPSIN M 70W | Chem. Std. | 1570 | BDF | | |
| 5 | SERENADE | Bio. Std. | 1400* | ACE | 274 | 373 |
| | OPTIMUM | | | | | |
| | TOPSIN M 70W | Chem. Std. | 1570 | BDF | | |
| 6 | PROLINE | Chem. Std. | 200 | ACE | 176 | 294 |
| | TOPSIN M 70W | Chem. Std. | 1570 | BDF | | |

*application rate in g/ha
⁺Indicates timing of applications (have letter/date)

Similar studies were performed in field trials in Georgia of peanut to determine the ability of the B. amyloliquefaciens RTI472 strain to prevent and/or ameliorate the effects of the natural occurance of the plant pathogens Southern White Mold and Leaf Spot caused by Cercospora/Cercosporidium in Peanut. The same application rates were used for RTI472 and SERENADE OPTIMUM as described above and the same format was used where separate applications of each of the strains were performed 6 times with 5 to 7 day intervals between applications. Stems were evaluated as in the white mold trial for snap bean. Canopy—Six, 2 foot sections per plot were evaluated as subsamples and the severity was determined by estimating the percentage of the foliage that showed symptoms from the entire canopy. The disease serverity was scored as number of hits per/1 m row for White Mold and Leaf Spot index was scored on a 1-10 rating. All peanuts were harvested, counted, weighted and separated in to marketable or diseased to determine yield per acre.

The results of the experiments are shown below for moderate disease pressue and for heavy disease pressure in Tables XIII and XIV, repsectively. RTI472 controlled peanut leafspot better than SERENADE OPTIMUM and the Untreated control. These results were observed in two trials one with moderate pressure and one with heavy pressure. RTI472 suppressed peanut southern white mold better than SERENADE OPTIMUM and the Untreated control. These results were observed in two trials one with moderate pressure and one with heavy pressure. No negative crop response was observed for RTI472 across the spray program which included six application timings. Peanut yields where both diseases were present in the field showed that treatment with RTI472 resulted in a higher yield response versus SERENADE OPTIMUM and the Untreated control.

TABLE XIII
Results of B. amyloliquefaciens RTI472 control
of Leaf Spot and Southern White Mold (moderate pressure)
in peanut as compared to SERENADE OPTIMUM.

| | | SERENADE | |
|---|---|---|---|
| Moderate Pressure | RTI472 | OPTIMUM | Check |
| Leaf Spot | 4.2 | 5.3 | 6 |
| Cercospora/Cercosporidium | | | |
| Southern White Mold | 10.7 | 11.2 | 18.8 |
| Yield lbs/Ac | 3933 | 3517 | 3479 |
| % Yield over control | 13% | 1% | n.a. |

TABLE XIV
Results of B. amyloliquefaciens RTI472 control
of Leaf Spot and Southern White Mold (heavy pressure)
in peanut as compared to SERENADE OPTIMUM.

| | | SERENADE | |
|---|---|---|---|
| Heavy Pressure | RTI472 | OPTIMUM | Check |
| Leaf Spot | 6.7 | 7.5 | 9 |
| Cercospora/Cercosporidium | | | |
| Southern White Mold | 12.6 | 17.2 | 22.7 |
| Yield lbs/Ac | 2454 | 2249 | 2195 |
| % Yield over control | 12% | 2.5% | n.a. |

### Example 10

### B. Amyloliquefaciens RTI472 Antagonism of Wheat Head Scab, Soybean Rust, and Alternaria Solani

Studies were performed in field trials in Georgia of wheat, soybean and tomato to determine the ability of the B. amyloliquefaciens RTI472 strain to prevent and/or ameliorate the effects of the plant pathogens wheat head scab, soybean rust, and Alternaria solani.

Applications were made for each of RTI472 and SERENADE OPTIMUM using an application rate of 1400 g/ha for SERENADE OPTIMUM and an application rate for RTI472 corresponding to the same colony forming units/ha as recommended for SERENADE OPTIMUM based on a 1400 g/ha application rate. Treatment applications were delivered to the crops with 5 to 7 day intervals between applications unless otherwise specified. The number of applications and timing of the first application depended on the particular crop and ranged from at the time of planting, a fews weeks after crop emergence, at the time of flowering, upon disease emergence, or just prior to expectation of disease emergence. The application sprayer was setup to deliver 20 gallons per acre (1891/ha). The individual plots were sprayed at a ground speed of 3 mph using a CO₂ backpack sprayer with twin flat fan nozzles (8003 type) and each nozzle was spaced 18 inches apart. The carrier to deliver the chemical was water mixed in a 2 liter bottle.

For wheat head scab, a single treatment application was made at the time of crop flowering. Three days after treatment, the plants were artificially infected with the head scab pathogen Gibberella zeae (also known as Fusarium graminearum). Disease severity was measured by determining the percentage of the wheat head affected by the head scab (bleaching of the spikelets). The percentage disease control is based on considering the diseased, non-treated control plants as 100%. The data are shown below in Table XV.

For soybean rust, 6 treatment applications were made. The initial application was delivered at the R1 stage of growth. This trial had a natural infestation. Disease severity was measured by evaluating stems as in the white mold trial. Canopy—six, 2 foot sections per plot were evaluated as subsamples and the severity was determined by estimating the percentage of the foliage that showed symptoms of rust from the entire canopy. The percentage disease control is based on considering the diseased, non-treated control plants as 100%. The data are shown below in Table XV.

For tomato, 2 treatment applications were made. Disease severity was measured by looking at the canopy and estimating the percentage of the foliage affected. The percentage disease control is based on considering the diseased, non-treated control plants as 100%. The data are shown below in Table XV.

TABLE XV
Results of B. amyloliquefaciens RTI472 disease
control of wheat head scab, soybean rust and Alternaria
on tomato as compared to SERENADE OPTIMUM.

| | Percent Control of | | |
|---|---|---|---|
| | Disease Severity | | |
| | | SERENADE | Disease Severity |
| Pathogen | RTI472 | OPTIMUM | in Control Plants |
| Wheat Head Scab | 50% bcd | 42% d | 85% |
| Soybean Rust | 68% a | 53% b | 44% |
| Alternaria on | 42% a | n.d. | 71% |
| Tomato | | | |

RTI472 controlled wheat head scab better than SERENADE OPTIMUM as measured by a percent of the Untreated control. The wheat scab trials had heavy severity/ pressure at 85% in the Untreated Control. RTI472 controlled soybean rust better than SERENADE OPTIMUM as measured by a percent of the Untreated control. The soybean rust trials had moderate severity/ pressure at 44% in the Untreated Control. No negative crop response was noted with RTI472 across the treatment application program which included one application in wheat, two applications in tomato, and six applications in soybeans.

### Example 11

### B. amyloliquefaciens RTI472 Antagonism of Bacterial Spot Tomato Disease (Xanthomonas) in Tomatoes in Field Trials in Georgia

Studies were performed in field trials of tomato to determine the ability of the B. amyloliquefaciens RTI472 strain to prevent and/or ameliorate the effects of the plant pathogen Bacterial Spot Tomato Disease (Xanthomonas).

A total of 4 applications to the crop were made with 5 to 7 day intervals between applications. In the case of the programs 4 and 5, which are combining the application of a biological with a chemical active ingredient, the first and third applications were made with the biological, while the second and fourth applications were made with the chemical.

Formulations:

SERENADE OPTIMUM was applied at a rate of 1400 g/ha, corresponding to 1.8×10⁺¹³ CFU/ha.

B. amyloliquefaciens RTI472 spores were in Spent Fermentation Broth (SFB) with added yeast extract and the application rate corresponded to the same colony forming units/ha as recommended for SERENADE OPTIMUM based on a 1400 g/ha application rate with yeast extract ranging from about 0.01% to 0.2%.

TACTIC (LOVELAND PRODUCTS, INC) was applied at a concentration of 0.1875% v/v.

KOCIDE 3000 (DUPONT USA) was applied at a rate of 1850 g a.i./ha (Copper Hydroxide fungicide).

BRAVO WEATHER STIK (SYNGENTA CROP PROTECTION, INC) was applied at a rate of 2240 g a.i./ha (Chlorothalonil).

The experimental design was as follows: Untreated control, RTI472+TACTIC, SERENADE OPTIMUM+TACTIC, RTI472+KOCIDE 3000+TACTIC, SERENADE OPTIMUM+KOCIDE 3000+TACTIC, and BRAVO WEATHER STIK+KOCIDE 3000+TACTIC.

Treatment Application Method:

Four separate treatment applications were delivered to the crop with a 5 to 7 interval between each application. The application sprayer was setup to deliver 40 gallons per acre. The individual plots were sprayed at a ground speed of 3 mph using a CO₂ backpack sprayer with cone nozzles and each nozzle was spaced 12 inches apart. The carrier to deliver the chemical was water mixed in a 2.5 liter bottle.

The disease severity was measured by evaluatining the canopy. The mean percent of disease serverity was evaluated in the middle of the plants for each of the treatments. The percentage disease control is based on considering the diseased, non-treated control plants as 100%. The data are shown below in Table XVI. The treatments included: Untreated control, RTI472+TACTIC, SERENADE OPTIMUM+TACTIC, RTI472+KOCIDE 3000+TACTIC, SERENADE OPTIMUM+KOCIDE 3000+TACTIC, and BRAVO WEATHER STIK+KOCIDE 3000+TACTIC. The best control of Bacterial Spot Tomato Disease (Xanthomonas) on tomatoes was observed for B. amyloliquefaciens RTI472 as a stand alone or in the program with KOCIDE, and outperformed the program based on the use of BRAVO WEATHER STIK (Chlorothalonil; SYNGENTA) combined with KOCIDE 3000.

TABLE XVI
Results of B. amyloliquefaciens RTI472 control of Bacterial
Spot Tomato Disease (Xanthomonas) in tomatoes as compared
to SERENADE OPTIMUM and chemical active agents.

| | | Mean % |
|---|---|---|
| | Severity of Bacterial spot (mid canopy) | disease |
| 1 | Untreated control | 78 a |
| 2 | RTI 472 + TACTIC | 25 c |
| 3 | SERENADE OPTIMUM + TACTIC | 39 b |
| 4 | RTI 472 + KOCIDE 3000 + TACTIC | 24 c |
| 5 | SERENADE OPTIMUM + KOCIDE 3000 + | 43 b |
| | TACTIC | |
| 6 | BRAVO WEATHER STIK + KOCIDE 3000 + | 37 b |
| | TACTIC | |

### Example 12

### Effects of Coating Corn Seed with Bacillus Amyloliquefaciens RTI472 Isolate

Experiments were performed to determine the effect of coating corn seed with spores of the B. amyloliquefaciens RTI472 strain in addition to a typical chemical control. The effects on time to plant emergence, plant stand, plant vigor, and grain yield were measured for an average of three field trials in Wisconsin. Experiments were performed as described below.

Formulations:

A B. amyloliquefaciens RTI472 spore concentrate (1.0×10⁺¹⁰ cfu/ml) in water was applied at an amount of 1.0×10⁺⁵ cfu/seed.

MAXIM (SYNGENTA CROP PROTECTION, INC) was applied to seed at 0.0064 mg Al/kernel (fludioxonil).

Metalaxyl was applied to seed at 0.005 mg Al/kernel.

PONCHO 250 (BAYER CROP SCIENCE) was applied to seed at 0.25 mg Al/kernel (Clothianidin).

Treatment Application Method:

Seed treatment was performed by mixing corn seeds with a solution containing spores of B. amyloliquefaciens RTI472 and chemical control MAXIM+Metalaxyl+PONCHO 250 that resulted in an average of 1×10⁵ cfu per seed and the chemical active ingredients at the label-indicated concentrations as detailed above. The experiment was performed with untreated seed and seed treated with the chemical control alone as controls. The untreated seed and each of the treated corn seed were planted in three separate field trials in Wisconsin and analyzed by length of time to plant emergence, plant stand, plant vigor, and grain yield in bushels/acre. Using an average of the data from the three field trials, addition of the chemical control as compared to untreated seed resulted in a statistically significant increase in each of time to plant emergence, plant stand, plant vigor, and grain yield. Inclusion of the B. amyloliquefaciens RTI472 in the seed treatment as compared to the seed treated with chemical control alone did not have a statistically significant effect on time to plant emergence, plant stand, or plant vigor, but did result in an increase of 6 bushels/acre of grain (from 231 to 237 bushels/acre) representing a 2.6% increase in grain yield.

A related trial was performed as described above, except that the corn plants were challenged separately with the pathogens Rhizoctonia and Fusarium graminearum. Disease severity was rated by visual inspection on a scale of 1 to 5. Treatment of the seed with B. amyloliquefaciens RTI472 as compared to seed treated with chemical control alone resulted in a statistically significant decrease in disease severity for Rhizoctonia and for naturally occurring diseases.

### Example 13

### B. amyloliquefaciens RTI472 Antagonism of Brownish Grey Mildew (Botrytis cinerea) in Strawberry in Field Trials in Italy and Spain

Studies were performed in field trials of strawberry to determine the ability of the B. amyloliquefaciens RTI472 strain to prevent and/or ameliorate the effects of the plant pathogen Brownish Grey Mildew (Botrytis cinerea).

A total of 4 applications to the crop were made with 7 day intervals between applications.

Formulations:

SERENADE MAX was applied at a rate of 4000 g/ha, corresponding to 2.0×10⁺¹⁴ CFU/ha of Bacillus subtilis strain QST713.

B. amyloliquefaciens RTI472 spores were in Spent Fermentation Broth (SFB) with added yeast extract and the application rate was 2.0×10⁺¹³ CFU/ha with yeast extract ranging from about 0.01% to 0.2%. In addition SILWET L77 (HELENA CHEMICAL), a nonionic organosilicone surfactant, was added at a rate of 0.15 liter per 100 liter spray solution.

The first two applications to the crop were made with SWITCH (cyprodinil 375 g/kg plus fludioxonil 250 g/kg; SYNGENTA CROP PROTECTION, INC) at a rate of 0.8 kg/ha, followed by two applications of SIGNUM (boscalid 267 g/kg plus pyraclostrobin 67 g/kg; BAYER CROP SCIENCE, INC) at a rate of 1.8 kg/ha. This is referred to herein as the “FARMER's program”.

The experimental design was as follows: Untreated control (UTC), FARMER's program, RTI472+SILWET L77, and SERENADE MAX.

Treatment Application Method:

Four independent field trials were performed, each with 4 replicates, and the results of the trials were combined and presented as average results. Four separate treatment applications were delivered to the crop with a 7 day interval between each application. Three days before the start of the treatments, all plots, including the untreated control, were treated with SWITCH to suppress initial disease development. The application sprayer was setup to deliver 53 to 107 gallons per acre depending on crop density at application. The individual plots were sprayed at a ground speed of 0.56 mph (0.25 m/s or 0.9 km/h) using a CO₂ backpack sprayer with cone nozzles and each nozzle was spaced 2 inches apart (5.5 cm).

All strawberries were harvested, counted, weighted and separated in to marketable or diseased to determine yield. The disease incidence (% of fruit affected by Brownish Grey Mildew) was measured by evaluating the fruits for each of the treatments at harvest on 6 separate dates and expressed as “Area Under Disease Pressure Curve” (AUDPC). The disease incidence and % increase in yield versus the untreated control are shown below in Table XVIII. The disease incidence in the UTC as a function of time is shown in the graphs below (FIG. 13, and shows that the disease pressure during the trials reaching highest disease pressure of about 20% to 45% of fruits infested.

The results in Table XVIII below show that improved control of Brownish Grey Mildew (Botrytis cinerea) on strawberry over the untreated control was observed for all three treatments, B. amyloliquefaciens RTI472, SERENADE MAX, and the FARMER's program, with a slightly higher numerical increase of yield for the treatment with RTI472.

TABLE XVIII
Results of B. amyloliquefaciens RTI472 control of
Brownish Grey Mildew (Botryotinia fuckeliana) on strawberry
as compared to SERENADE MAX and the FARMER's program
based on chemical active agents. The results are the
average of four independent field trials.

| | | Average % | % Yield | Statistical |
|---|---|---|---|---|
| | Severity of Brownish | disease as | increase | relevance |
| | Grey Mildew | AUDPC | over UTC | (P of 0.1) |
| 1 | Untreated control (UTC) | 19.1 | 0 | a |
| 2 | FARMER's program | 13.2 | 11.5 | b |
| 3 | RTI472 + SILWET L77 | 12.9 | 15.5 | b |
| 4 | SERENADE MAX | 12.7 | 14.9 | b |

### Example 14

### B. Amyloliquefaciens RTI472 Antagonism of Powdery Mildew in Summer Squash

Studies were performed in field trials of summer squash to determine the ability of the B. amyloliquefaciens RTI472 strain as a stand-alone or as a mixture with FRACTURE to prevent and/or ameliorate the effects of powdery mildew caused by Golovinomyces cichoracearum (ERYSCI) in summer squash. FRACTURE (CONSUMO EM VERDE (CEV), BIOTECNOLOGIA DAS PLANTAS S.A., PORTUGAL) is a plant extract-based formulation containing 20% BLAD polypeptide as active ingredient. BLAD polypeptide is a fragment of a naturally occurring seed storage protein in sweet lupine (Lupinus albus) that acts on susceptible fungal pathogens by causing damage to the fungal cell wall and disrupting the inner cell membrane.

In total, 6 applications were made with 5 to 7 day intervals between applications for each product using the rates as indicated in Table XIX below unless otherwise specified. The timing of the first application was made just prior to expectation of disease emergence. In the case of RTI472 (** application rate of 1.8×10⁺¹³ CFU/ha), the application rate corresponded to the same colony forming units/ha as recommended for SERENADE OPTIMUM based on a 1400 g/ha application rate.

Formulations:

SERENADE OPTIMUM (BAYER CROP SCIENCE, INC) was applied at a rate of 1400 g/ha, corresponding to 1.8×10⁺¹³ CFU/ha.

FRACTURE (CONSUMO EM VERDE (CEV), BIOTECNOLOGIA DAS PLANTAS S.A., PORTUGAL) was applied at 672 g a.i./ha (BLAD).

B. amyloliquefaciens RTI472 spores plus Spent Fermentation Broth (SFB) with added yeast extract at 0.15%, sucrose at 0.2%, MgSO₄ at 0.02%, and CaCl₂ at 0.002%, and the application rate corresponded to the same colony forming units/ha as recommended for SERENADE OPTIMUM based on a 1400 g/ha application rate.

BRAVO WEATHER STIK (SYNGENTA CROP PROTECTION, INC) product was applied at a rate of 1680 g a.i./ha (Chlorothalonil).

LUNA EXPERIENCE (BAYER CROP SCIENCE, INC) was applied at a rate of 500 g a.i./ha (Fluopyram plus Tebuconazole fungicide).

TACTIC (LOVELAND PRODUCTS, INC) was applied at a concentration of 0.1875% v/v.

The experimental design was as follows: Untreated control, FRACTURE, SERENADE OPTIMUM+TACTIC, BRAVO WEATHER STIK+TACTIC, LUNA EXPERIENCE+TACTIC, RTI472+TACTIC, RTI472+FRACTURE+TACTIC.

Treatment Application Method: All applications were performed 6 times with 5 to 7 day intervals between applications unless otherwise specified. The application sprayer was setup to deliver 30 gallons per acre. The individual plots were sprayed at a ground speed of 4 mph using a CO₂ backpack sprayer with flat fan nozzles (8004 type) and each nozzle was spaced 18 inches apart.

The carrier to deliver the chemical was water mixed in a 2 liter bottle. For the application of RTI472 plus FRACTURE, both products were tank mixed and applied at the same concentration as the individual products.

Disease Scoring: Powdery mildew on summer squash was evaluated on the level of the top leafs. Disease severity, expressed as “Area Under Disease Pressure Curve” (AUDPC), was determined in function of severity (% leaf area affected) of powdery mildew on the upper leaf surface. Untreated plants were determined to have 100% disease incidence and 62.8% disease severity.

The results of the experiment are shown in Table XIX below. FRACTURE provided approximately 15% reduction of disease severity, while SERENADE OPTIMUM and RTI472 gave approximately 30% reduction of disease severity. The most significant reductions in disease severity were observed for BRAVO WEATHER STIK (45%) and LUNA EXPERIENCE (94%). Interestingly, when RTI472 and FRACTURE were applied together, a cumulative effect for disease control was observed, resulting in a reduction of 47% in disease severity. This is comparable to the reduction in disease severity for BRAVO WEATHER STIK (45%). Therefore, the product combination based on B. amyloliquefaciens RTI472 and FRACTURE could be used as an alternative to the chlorothalonil fungicide in a program for the control powdery mildew on cucurbits.

TABLE XIX
Results of B. amyloliquefaciens RTI472 control of powdery mildew caused by
Golovinomyces cichoracearum (ERYSCI) in summer squash as compared to
SERENADE OPTIMUM, FRACTURE and other chemical active agents.

| | | | | | AUDPC | |
|---|---|---|---|---|---|---|
| | | | | | Canopy | |
| | | | | | Mildew | Statistical |
| No. | Name | Description | Rate g/ha | Code⁺ | severity | significance |
| 1 | UNTREATED | N.A. | N.A. | N.A. | 310 | a |
| 2 | FRACTURE | Biological | 672* | ABCDEF | 269 | abc |
| 3 | SERENADE | Bio. Std. | 1.8 × 10⁺¹³** | ABCDEF | 208 | bcd |
| | OPTIMUM | | | | | |
| 4 | BRAVO WEATHER | Protectant - | 1680* | ABCDEF | 169 | d |
| | STIK | Chem. Std. | | | | |
| 5 | LUNA EXPERIENCE | Curative - | 500* | ABCDEF | 19 | e |
| | | Chem. Std. | | | | |
| 6 | RTI472 | Biological | 1.8 × 10⁺¹³** | ABCDEF | 197 | cd |
| 7 | RTI472 | Biological | 1.8 × 10⁺¹³** | ABCDEF | 164 | d |
| | FRACTURE | Biological | 1570 | ABCDEF | | |

*application rate in g a.i./ha
**application rate in cfu/ha
⁺Indicates timing of applications (have letter/date)
N.A. not applicable

### Example 15

### Identification of New Metabolites produced by Bacillus amyloliquefaciens RTI472 Isolate

It has been previously reported that five classes of Fengycin-type metabolites and Dehydroxyfengycin-type metabolites are produced by microbial species including Bacillus amyloliquefaciens (see, for example, Li, Xing-Yu, et al. , 2013, J. Microbiol. Biotechnol. 23(3), 313-321; Pecci Y, et al. 2010 Mass Spectrom., 45(7):772-77). These metabolites, cyclic lipopeptides, are cyclic peptide molecules that also contain a fatty acid group. The five classes of Fengycin- and Dehydroxyfengycin-type metabolites are referred to as A, B, C, D and S. The backbone structure of these metabolites as well as the specific amino acid sequence for each of the five classes is shown in FIG. 13.

The Fengycin- and Dehydroxyfengycin-type metabolites produced by Bacillus amyloliquefaciens RTI472 were analyzed using UHPLC-TOF MS. The molecular weights of the Fengycin-type metabolites produced by the RTI472 strain after both 3 and 6 days growth in 869 medium at 30° C. were compared to the theoretical molecular weights expected for the Fengycin- and Dehydroxyfengycin-type metabolites. In addition, to determine the amino acid composition of the various Fengycin-type metabolites produced by the RTI472 strain, peptide sequencing using LC-MS-MS was performed on each of the Fengycin-type metabolites previously identified via UHPLC-TOF MS. In this manner, it was determined that Bacillus amyloliquefaciens RTI472 produces Fengycin A, B, C, D, and S and Dehydroxyfengycin A, B, C, D, and S. Surprisingly, in addition to these known compounds, it was determined that the RTI472 strain also produces several classes of previously unidentified derivatives of these compounds.

For example, it was determined that the Bacillus amyloliquefaciens RTI472 strain produces Fengycin-like and Dehydroxyfengycin-like compounds where the L-isoleucine at position 8 of the cyclic peptide chain (referred to as X₃ in FIG. 14) is replaced by L-methionine. The new classes of Fengycin and Dehydroxyfengycin are referred to herein as MA, MB and MC, referring to derivatives of classes A, B and C in which the L-isoleucine at X₃ in FIG. 14 has been replaced by L-methionine. The newly identified molecules are shown in bold in FIG. 14 and in Table XX below. As noted in Table XX, the Dehydroxyfengycin MC compound was not observed in the culture of the RTI472 strain.

It was further determined that the Bacillus amyloliquefaciens RTI472 strain produces an additional class of previously unidentified Fengycin (and putatively Dehydroxyfengycin) metabolites. In this class, the L-isoleucine of Fengycin B (position X₃ in FIG. 13) is replaced by L-homo-cysteine (Hcy). This previously unidentified metabolite is referred to herein as Fengycin H.

It was further determined that the RTI472 strain produces an additional class of previously unidentified Fengycin and Dehydroxyfengycin metabolites. In this class, the amino acid at position 4 of the cyclic peptide backbone structure (position X₁ in FIG. 14) is replaced by L-isoleucine. These previously unidentified metabolites are referred to herein as Fengicin I and Dehydroxyfengicin I and are shown in bold in FIG. 14 and in Table XX.

A summary of the amino acid sequences for the previously reported Fengycin- and Dehydroxyfengycin-type lipopeptides and the newly identified metabolites is provided in Table XX below.

TABLE XX
Summary of UHPLC-TOF MS identification of Fengycin-type lipopeptides in Bacillus
amyloliquefaciens RTI472.

| | | | | | | Theoretical C16 | Theoretical | |
|---|---|---|---|---|---|---|---|---|
| | | | | | Ring | Molecular | C16 | Observed |
| Homolog | X₁ | X₂ | X₃ | R | Mass | Formula | [M + H]⁺ | RTI472 |
| Fengycin A | Ala | Thr | Ile | OH | 1080.6 | C₇₂H₁₁₀N₁₂O₂₀ | 1463.8 | C14, C15, C16, C17 |
| Fengycin B | Val | Thr | Ile | OH | 1108.7 | C₇₄H₁₁₄N₁₂O₂₀ | 1491.8 | C14, C15, C16, C17 |
| Fengycin C | Aba | Thr | Ile | OH | 1094.6 | C₇₃H₁₁₂N₁₂O₂₀ | 1477.8 | C14, C15, C16, C17 |
| Fengycin D | Val | Thr | Val | OH | 1094.6 | C₇₃H₁₁₂N₁₂O₂₀ | 1477.8 | C14, C15, C16, C17 |
| Fengycin S | Val | Ser | Ile | OH | 1094.6 | C₇₃H₁₁₂N₁₂O₂₀ | 1477.8 | C14, C15, C16, C17 |
| Fengycin MA | Ala | Thr | Met | OH | 1098.7 | C₇₁H₁₀₈N₁₂O₂₀S | 1481.8 | C14, C15, C16, C17 |
| Fengycin MB | Val | Thr | Met | OH | 1126.8 | C₇₃H₁₁₂N₁₂O₂₀S | 1509.8 | C14, C15, C16, C17 |
| Fengycin MC | Aba | Thr | Met | OH | 1112.7 | C₇₂H₁₁₀N₁₂O₂₀S | 1495.8 | C14, C15, C16, C17 |
| Fengycin H | Val | Thr | Hcy | OH | 1112.7 | C₇₂H₁₁₀N₁₂O₂₀S | 1495.8 | C14, C15, C16, C17 |
| Fengycin I | Ile | Thr | Ile | OH | 1122.8 | C₇₅H₁₁₆N₁₂O₂₀ | 1505.8 | C16, C17 |
| Dehydroxyfengycin A | Ala | Thr | Ile | H | 1080.6 | C₇₂H₁₁₀N₁₂O₁₉ | 1447.8 | C15, C16, C17 |
| Dehydroxyfengycin B | Val | Thr | Ile | H | 1108.7 | C₇₄H₁₁₄N₁₂O₁₉ | 1475.8 | C14, C15, C16, C17 |
| Dehydroxyfengycin C | Aba | Thr | Ile | H | 1094.6 | C₇₃H₁₁₂N₁₂O₁₉ | 1461.8 | C14, C15, C16, C17 |
| Dehydroxyfengycin D | Val | Thr | Val | H | 1094.6 | C₇₃H₁₁₂N₁₂O₁₉ | 1461.8 | C14, C15, C16, C17 |
| Dehydroxyfengycin S | Val | Ser | Ile | H | 1094.6 | C₇₃H₁₁₂N₁₂O₁₉ | 1461.8 | C14, C15, C16, C17 |
| Dehydroxyfengycin | Ala | Thr | Met | H | 1098.7 | C₇₁H₁₀₈N₁₂O₁₉S | 1465.7 | C17 |
| MA | | | | | | | | |
| Dehydroxyfengycin | Val | Thr | Met | H | 1126.8 | C₇₃H₁₁₂N₁₂O₁₉S | 1493.8 | C16, C17 |
| MB | | | | | | | | |
| Dehydroxyfengycin | Aba | Thr | Met | H | 1112.7 | C₇₂H₁₁₀N₁₂O₁₉S | 1479.8 | Not observed |
| MC | | | | | | | | |
| Dehydroxyfengycin H | Val | Thr | Hcy | H | 1112.7 | C₇₂H₁₁₀N₁₂O₁₉S | 1479.8 | Not observed |
| Dehydroxyfengycin I | Ile | Thr | Ile | H | 1122.8 | C₇₅H₁₁₆N₁₂O₁₉ | 1489.9 | C14, C15, C16 |

### Example 16

### Antimicrobial Activity of Isolated Lipopeptide Metabolites of RTI472

Antagonistic lipopeptides from B. amyloliquefaciens strain RTI472 were isolated from RTI472 spent fermentation broth and shown to retain their activity.

In this experiment, the Bacillus amyloliquefaciens culture supernatant was acidified to pH 2 according to the procedure described in Smyth, T J P et al., 2010, “Isolation and Analysis of Lipopeptides and High Molecular Weight Biosurfactants.” In: Handbook of Hydrocarbon and Lipid Microbiology, K. N. Timmis (Editor). pp 3687-3704. The recovery of the lipopeptides was analyzed by UHPLC-TOF MS, and their antagonistic activity against Botrytis cinerea and Fusarium graminearum were tested.

The RTI472 was cultured in M2 sporulation medium for six days at 30° C., and the spent fermentation broth (472-SFB) was centrifuged at 18,514 g for 20 min to remove the spores. The supernatant was subsequently acidified to pH 2.0 by addition of concentrated HCl, and overnight precipitated at 4° C. The sample was subsequently centrifuged at 18,514 g for 20 min to obtain the solid crude lipopeptides. The pellet was lyophilized overnight, dissolved in the original volume of M2 medium, and analyzed by LCMS. The masses of iturins (C14, C15, C16), surfactins (C12, C13, C14, C15, C16, C17), and fengycins (A, B, C, D, S) were extracted, integrated, and summed up to compare relative abundance of the lipopeptides from each sample. FIG. 15 is a graph showing the percentage of recovered lipopeptides from the RTI472 spent fermentation broth (SFB) after the acid precipitation. The terms “472-AP-Pellet” and “472-AP-Supernatant” refer to the resuspended pellet and supernatant, respectively, obtained after acid precipitation of the centrifuged SFB. The results in the graph in FIG. 15 show that 83% of the total amount of lipopeptides was recovered by acid precipitation. For iturin 65% was precipitated, while 26% of the iturn was not recovered via the acid precipitation method. Surfactin and fengycin were 100% recovered using acid precipitation. To confirm that the LCMS results correlated with antagonistic activity, a bioassay was performed with the same samples analyzed by LCMS. For the bioassay, 20 μl of Botrytis cinerea or Fusarium graminearum inoculum was spotted in the middle of plate with 472-AP-Pellet sample spotted in 10 μl, 20 μl, and 40 μl aliquots. The antifungal activity was checked and imaged after 5 days or 7 days incubation at 30° C. for Botrytis cinerea and Fusarium graminearum plates, respectively. The results are shown in the images of the plate assay in FIGS. 16A-16D. FIGS. 16A and 16B are images of Botrytis cinerea spotted on 869 agar plates with A) RTI472 spent fermentation broth (472-SFB); and B) resuspended pellet material obtained after acid precipitation plus centrifugation (472-AP-Pellet). FIGS. 16C and 16D are images of Fusarium graminearum spotted on 869 agar plates with C) RTI472 spent fermentation broth (472-SFB); and D) resuspended pellet material obtained after acid precipitation plus centrifugation (472-AP-Pellet). For positive controls, 10 μl of cell culture of RTI472 was spotted on the left side of each plate next to the fungus. The results show that the acid precipitated sample (472-AP-Pellet) has a similar level of antagonistic activity as the starting spent fermentation broth against both Botrytis cinerea and Fusarium graminearum. The bioassay results are well correlated with the LCMS data in FIG. 15.

### REFERENCES

All publications, patent applications, patents, and other references mentioned in the specification are herein incorporated by reference in their entireties.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising a biologically pure culture of Bacillus amyloliquefaciens RTI472 deposited as ATCC No. PTA-121166, or a mutant thereof having all the identifying characteristics thereof, for application to a plant for one or both of benefiting plant growth or conferring protection against a pathogenic infection in a susceptible plant.

2. The composition of claim 1, wherein the composition is in the form of a liquid, a dust, a dry wettable powder, a spreadable granule, or a dry wettable granule.

3. The composition of claim 1, wherein the composition is in the form of a liquid and the Bacillus amyloliquefaciens RTI472 is present at a concentration of from about 1.0×10⁸ CFU/ml to about 1.0×10¹² CFU/ml.

4. The composition of claim 1, wherein the composition is in the form of a dust, a dry wettable powder, a spreadable granule, or a dry wettable granule and the Bacillus amyloliquefaciens RTI472 is present in an amount of from about 1.0×10⁸ CFU/g to about 1.0×10¹² CFU/g.

5. The composition of claim 1, wherein the composition is in the form of an oil dispersion and the Bacillus amyloliquefaciens RTI472 is present at a concentration of from about 1.0×10⁸ CFU/ml to about 1.0×10¹² CFU/ml.

6. The composition of claim 1, wherein the Bacillus amyloliquefaciens RTI472 is in the form of spores or vegetative cells.

7. The composition of claim 1, further comprising one or a combination of a carrier, a surfactant, a dispersant, or a yeast extract.

8. A plant seed coated with a composition comprising:
spores of a biologically pure culture of Bacillus amyloliquefaciens RTI472 deposited as ATCC No. PTA-121166, or a mutant thereof having all the identifying characteristics thereof, present in an amount suitable to benefit plant growth and/or to confer protection against a pathogenic infection in a susceptible plant.

9. The plant seed of claim 8, wherein the composition comprises an amount of Bacillus amyloliquefaciens spores from about 1.0×10² CFU/seed to about 1.0×10⁹ CFU/seed.

10. The plant seed of claim 8, wherein the composition further comprises one or a combination of a microbial, a biological, or a chemical insecticide, fungicide, nematicide, bacteriocide, or plant growth regulator present in an amount suitable to benefit plant growth and/or to confer protection against a pathogenic infection in a susceptible plant.

11. The plant seed of claim 10, wherein the fungicide is one or more of benzovindiflupyr, anitiperonosporic, ametoctradin, amisulbrom, copper salts including copper hydroxide, copper oxychloride, copper sulfate, and copper persulfate, boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens including strains QST 713, FZB24, MB1600, and D747, extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, estract from Reynoutria sachalinensis, phosphorous acid and salts, teclofthalam, triazoxide, pyriofenone, organic oils, potassium bicarbonate, chlorothalonil, fluoroimide; azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; strobilurins, including azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl(2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(443-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1 -methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1 H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide,N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; and other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenylacetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

12. The plant seed of claim 10, wherein the fungicide is one or a combination of fluopyram, tebuconazole, chlorothalonil, thiophanate-methyl, prothioconazole, fludioxonil, metalaxyl, or copper hydroxide.

13. The plant seed of claim 10, wherein the insecticide is one or a combination of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

14. The plant seed of claim 10, wherein the insecticide comprises bifenthrin.

15. A composition for one or both of benefiting plant growth or conferring protection against pathogenic infection in a susceptible plant, the composition comprising:
a biologically pure culture of Bacillus amyloliquefaciens RTI472 deposited as ATCC No. PTA-121166, or a mutant thereof having all the identifying characteristics thereof, in an amount suitable to benefit plant growth and/or to confer protection against pathogenic infection in the susceptible plant; and
one or a combination of a microbial, a biological, or a chemical insecticide, fungicide, nematicide, bacteriocide, herbicide, plant extract, plant growth regulator, or fertilizer, in an amount suitable to benefit plant growth and/or to confer protection against pathogenic infection in the susceptible plant.

16. The composition of claim 15, wherein the composition is in the form of a liquid and the Bacillus amyloliquefaciens RTI472 is present at a concentration of from about 1.0×10⁸ CFU/ml to about 1.0×10¹² CFU/ml.

17. The composition of claim 15, wherein the composition is in the form of a dust, a dry wettable powder, a spreadable granule, or a dry wettable granule and the Bacillus amyloliquefaciens RTI472 is present in an amount of from about 1.0×10⁸ CFU/g to about 1.0×10¹² CFU/g.

18. The composition of claim 15, wherein the Bacillus amyloliquefaciens RTI472 is in the form of spores or vegetative cells.

19. The composition of claim 15, wherein the fungicide is one or more of benzovindiflupyr, anitiperonosporic, ametoctradin, amisulbrom, copper salts including copper hydroxide, copper oxychloride, copper sulfate, and copper persulfate, boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens including strains QST 713, FZB24, MB1600, and D747, extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, estract from Reynoutria sachalinensis, phosphorous acid and salts, teclofthalam, triazoxide, pyriofenone, organic oils, potassium bicarbonate, chlorothalonil, fluoroimide; azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; strobilurins, including azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl(2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide, N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; and other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds:
dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

20. The compositon of claim 15, wherein the fungicide is one or a combination of fluopyram, tebuconazole, chlorothalonil, thiophanate-methyl, prothioconazole, fludioxonil, metalaxyl, or copper hydroxide.

21. The compositon of claim 15, wherein the insecticide is one or a combination of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

22. The compositon of claim 15, wherein the insecticide comprises bifenthrin and the composition is in a formulation compatible with a liquid fertilizer.

23. A product comprising:
a first composition comprising a biologically pure culture of Bacillus amyloliquefaciens RTI472 deposited as ATCC No. PTA-121166, or a mutant thereof having all the identifying characteristics thereof;
a second composition comprising one or a combination of a microbial, a biological, or a chemical insecticide, fungicide, nematicide, bacteriocide, herbicide, plant extract, plant growth regulator, or fertilizer, wherein the first and second compositions are separately packaged, and wherein each composition is in an amount suitable for one or both of benefiting plant growth or conferring protection against a pathogenic infection in a susceptible plant; and
instructions for delivering in an amount suitable to benefit plant growth, a combination of the first and second compositions to: foliage of the plant, bark of the plant, fruit of the plant, flowers of the plant, seed of the plant, roots of the plant, a cutting of the plant, a graft of the plant, callus tissue of the plant; soil or growth medium surrounding the plant; soil or growth medium before sowing seed of the plant in the soil or growth medium; or soil or growth medium before planting the plant, the plant cutting, the plant graft, or the plant callus tissue in the soil or growth medium.

24. The product of claim 23, wherein the fungicide is one or more of benzovindiflupyr, anitiperonosporic, ametoctradin, amisulbrom, copper salts including copper hydroxide, copper oxychloride, copper sulfate, and copper persulfate, boscalid, thiflumazide, flutianil, furalaxyl, thiabendazole, benodanil, mepronil, isofetamid, fenfuram, bixafen, fluxapyroxad, penflufen, sedaxane, coumoxystrobin, enoxastrobin, flufenoxystrobin, pyraoxystrobin, pyrametostrobin, triclopyricarb, fenaminstrobin, metominostrobin, pyribencarb, meptyldinocap, fentin acetate, fentin chloride, fentin hydroxide, oxytetracycline, chlozolinate, chloroneb, tecnazene, etridiazole, iodocarb, prothiocarb, Bacillus subtilis syn., Bacillus amyloliquefaciens including strains QST 713, FZB24, MB1600, and D747, extract from Melaleuca alternifolia, extract from Lupinus albus doce, BLAD polypeptide, pyrisoxazole, oxpoconazole, etaconazole, fenpyrazamine, naftifine, terbinafine, validamycin, pyrimorph, valifenalate, fthalide, probenazole, isotianil, laminarin, estract from Reynoutria sachalinensis, phosphorous acid and salts, teclofthalam, triazoxide, pyriofenone, organic oils, potassium bicarbonate, chlorothalonil, fluoroimide; azoles, including bitertanol, bromuconazole, cyproconazole, difenoconazole, diniconazole, enilconazole, epoxiconazole, fluquinconazole, fenbuconazole, flusilazole, flutriafol, hexaconazole, imibenconazole, ipconazole, metconazole, myclobutanil, penconazole, propiconazole, prothioconazole, simeconazole, triadimefon, triadimenol, tebuconazole, tetraconazole, triticonazole, prochloraz, pefurazoate, imazalil, triflumizole, cyazofamid, benomyl, carbendazim, thia-bendazole, fuberidazole, ethaboxam, etridiazole and hymexazole, azaconazole, diniconazole-M, oxpoconazol, paclobutrazol, uniconazol, 1-(4-chloro-phenyl)-2-([1,2,4]triazol-1-yl)-cycloheptanol and imazalilsulfphate; strobilurins, including azoxystrobin, dimoxystrobin, enestroburin, fluoxastrobin, kresoxim-methyl, methominostrobin, orysastrobin, picoxystrobin, pyraclostrobin, trifloxystrobin, enestroburin, methyl (2-chloro-5-[1-(3-methylbenzyloxyimino)ethyl]benzyl)carbamate, methyl(2-chloro-5-[1-(6-methylpyridin-2-ylmethoxyimino)ethyl]benzyl)carbamate and methyl 2-(ortho-(2,5-dimethylphenyloxymethylene)-phenyl)-3-methoxyacrylate, 2-(2-(6-(3-chloro-2-methyl-phenoxy)-5-fluoro-pyrimidin-4-yloxy)-phenyl)-2-methoxyimino-N-methyl-acetamide and 3-methoxy-2-(2-(N-(4-methoxy-phenyl)-cyclopropanecarboximidoylsulfanylmethyl)-phenyl)-acrylic acid methyl ester; carboxamides, including carboxin, benalaxyl, benalaxyl-M, fenhexamid, flutolanil, furametpyr, mepronil, metalaxyl, mefenoxam, ofurace, oxadixyl, oxycarboxin, penthiopyrad, isopyrazam, thifluzamide, tiadinil, 3,4-dichloro-N-(2-cyanophenyl)isothiazole-5-carboxamide, dimethomorph, flumorph, flumetover, fluopicolide (picobenzamid), zoxamide, carpropamid, diclocymet, mandipropamid, N-(2-(4-[3-(4-chlorophenyl)prop-2-ynyloxy]-3-methoxyphenyl)ethyl)-2-methanesulfonyl-amino-3-methylbutyramide, N-(2-(4-[3-(4-chloro-phenyl)prop-2-ynyloxy]-3-methoxy-phenyl)ethyl)-2-ethanesulfonylamino-3-methylbutyramide, methyl 3-(4-chlorophenyl)-3-(2-isopropoxycarbonyl-amino-3-methyl-butyrylamino)propionate, N-(4′-bromobiphenyl-2-yl)-4-difluoromethyl̂-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-difluoromethyl-2-methyl-thiazole-5-carboxamide, N-(3\4′-dichloro-4-fluorobiphenyl-2-yl)-3-difluoro-methyl-1-methyl-pyrazole-4-carboxamide, N-(3′,4′-dichloro-5-fluorobiphenyl-2-yl)-3-difluoromethyl-1-methylpyrazole-4-carboxamide, N-(2-cyano-phenyl)-3,4-dichloroisothiazole-5-carboxamide, 2-amino-4-methyl-thiazole-5-carboxanilide, 2-chloro-N-(1,1,3-trimethyl-indan-4-yl)-nicotinamide, N-(2-(1,3-dimethylbutyl)-phenyl)-1,3-dimethyl-5-fluoro-1H-pyrazole-4-carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-yl)-3-trifluoromethyl-1-methyl-1H-pyrazole-4-carboxamide, N-(3′,4′-dichloro-5-fluoro-biphenyl-2-yl)-3-trifluoromethyl-1-methyl-1H-pyrazole-4-carboxamide, N-(3′,5-difluoro-4′-methyl-biphenyl-2-yl)-3-difluoromethyl-1-methyl-1H-pyrazole-4-carboxamide, N-(3′,5-difluoro-4′-methyl-biphenyl-2-yl)-3-trifluoromethyl-1-methyl-1H-pyrazole-4-carboxamide, N-(cis-2-bicyclopropyl-2-yl-phenyl)-3-difluoromethyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-ethyl-3,5-5-trimethyl-cyclohexyl)-3-formylamino-2-hydroxy-benzamide, oxytetracyclin, silthiofam, N-(6-methoxy-pyridin-3-yl) cyclopropanecarboxamide, 2-iodo-N-phenyl-benzamide, N-(2-bicyclo-propyl-2-yl-phenyl)-3-difluormethyl-1-methylpyrazol-4-ylcarboxamide, N-(3′,4′,5′-trifluorobiphenyl-2-yl)-1,3-dimethylpyrazol-4-ylcarboxamide, N-(3′,4′,5′-trifluorobiphenyl-2-yl)-1,3-dimethyl-5-fluoropyrazol-4-yl-carboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chloro-3-difluoromethyl-1-methylpyrazol-4-ylcarboxamide, N-(3′,4′,5′-trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-trifluoromethylpyrazol-4-ylcarboxamide, N-(3′,4′,5′-trifluorobiphenyl-2-yl)-5-fluoro-1-methyl-3-trifluoromethylpyrazol-4-ylcarboxamide, N-(3′,4′,5′-trifluorobiphenyl-2-yl)-5-chloro-1-methyl-3-trifluoromethylpyrazol-4-ylcarboxamide, N-(2′,4′,5′-trifluorobiphenyl-2-yl)-1,3-dimethylpyrazol-4-ylcarboxamide, N-(2′,4′,5′-trifluorobiphenyl-2-yl)-1,3-dimethyl-5-fluoropyrazol-4-ylcarboxamide, N-(2′,4′,5′-trifluorobiphenyl-2-yl)-5-chloro-1,3-dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl)-3-difluoromethyl-1-methylpyrazol-4-ylcarboxamide, N-(2′,4′,5′-trifluorobiphenyl-2-yl)-3-difluoromethyl-5-fluoro-1-methylpyrazol-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fluoro-1-methyl-3-trifluoromethylpyrazol-4-ylcarboxamide, N-(2′,4′,5′-trifluorobiphenyl-2-yl)-5-chloro-1-methyl-3-trifluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluorobiphenyl-2-yl)-1-methyl-3-trifluoromethyl-1H-pyrazole-4-carboxamide, N-(3′,4′-difluoro-3-fluorobiphenyl-2-yl)-1-methyl-S-difluoromethyl-1H-pyrazole-4-carboxamide, N-(3′-chloro-4′-fluoro-3-fluorobiphenyl-2-yl)-1-methyl-3-difluoromethyl-1H-pyrazole-4-carboxamide, N-(3′,4′-dichloro-4-fluorobiphenyl-2-yl)-1-methyl-3-trifluoromethyl-1H-pyrazole-4-carboxamide, N-(3′,4′-difluoro-4-fluorobiphenyl-2-yl)-1-methyl-S-trifluoromethyl-1H-pyrazole-4-carboxamide, N-(3′,4′-dichloro-4-fluorobiphenyl-2-yl)-1-methyl-3-difluoromethyl-1H-pyrazole-4-carboxamide, N-(3′,4′-difluoro-4-fluorobiphenyl-2-yl)-1-methyl-3-difluoromethyl-1H-pyrazole-4-carboxamide, N-(3′-chloro-4′-fluoro-4-fluorobiphenyl-2-yl)-1-methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-dichloro-5-fluorobiphenyl-2-yl)-1-methyl-S-difluoromethyl-1H-pyrazole-carboxamide, N-(3′,4′-difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-fluorobiphenyl-2-yl)-1-methyl-3-trifluoromethyl-1H-pyrazole-4-carboxamide,N-(4′-chloro-5-fluorobiphenyl-2-yl)-1-methyl-3-trifluoromethyl-1H-pyrazole-4-carboxamide, N-(4′-methyl-5-fluorobiphenyl-2-yl)-1-methyl-3-trifluoromethyl-1H-pyrazole-4-carboxamide, N-(4′-fluoro-5-fluorobiphenyl-2-yl)-1,3-dimethyl-1H-pyrazole-4-carboxamide, N-(4′-chloro-5-fluorobiphenyl-2-yl)-1,3-dimethyl-1H-pyrazole-4-carboxamide, N-(4′-methyl-5-fluorobiphenyl-2-yl)-1,3-dimethyl-1H-pyrazole-4-carboxamide, N-(4′-fluoro-6-fluorobiphenyl-2-yl)-1-methyl-3-trifluoromethyl-1H-pyrazole-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-yl]-3-difluoromethyl-1-methyl-1H-pyrazole-4-carboxamide and N-[4′-(trifluoromethylthio)-biphenyl-2-yl]-1-methyl-3-trifluoromethyl-1-methyl-1H-pyrazole-4-carboxamide; heterocyclic compounds, including fluazinam, pyrifenox, bupirimate, cyprodinil, fenarimol, ferimzone, mepanipyrim, nuarimol, pyrimethanil, triforine, fenpiclonil, fludioxonil, aldimorph, dodemorph, fenpropimorph, tridemorph, fenpropidin, iprodione, procymidone, vinclozolin, famoxadone, fenamidone, octhilinone, proben-azole, 5-chloro-7-(4-methyl-piperidin-1-yl)-6-(2,4,6-trifluorophenyl)-[1,2,4]triazolo[1,5-a]pyrimidine, anilazine, diclomezine, pyroquilon, proquinazid, tricyclazole, 2-butoxy-6-iodo-3-propylchromen-4-one, acibenzolar-S-methyl, captafol, captan, dazomet, folpet, fenoxanil, quinoxyfen, N,N-dimethyl-3-(3-bromo-6-fluoro-2-methylindole-1-sulfonyl)-[1 ,2,4]triazole-1-sulfonamide, 5-ethyl-6-octyl-[1,2,4]triazolo[1,5-a]pyrimidin-2,7-diamine, 2,3,5,6-tetrachloro-4-methanesulfonyl-pyridine, 3,4,5-trichloro-pyridine-2,6-di-carbonitrile, N-(1-(5-bromo-3-chloro-pyridin-2-yl)-ethyl)-2,4-dichloro-nicotinamide, N-((5-bromo-3-chloropyridin-2-yl)-methyl)-2,4-dichloro-nicotinamide, diflumetorim, nitrapyrin, dodemorphacetate, fluoroimid, blasticidin-S, chinomethionat, debacarb, difenzoquat, difenzoquat-methylsulphat, oxolinic acid and piperalin; carbamates, including mancozeb, maneb, metam, methasulphocarb, metiram, ferbam, propineb, thiram, zineb, ziram, diethofencarb, iprovalicarb, benthiavalicarb, propamocarb, propamocarb hydrochlorid, 4-fluorophenyl N-(1-(1-(4-cyanophenyl)-ethanesulfonyl)but-2-yl)carbamate, methyl 3-(4-chloro-phenyl)-3-(2-isopropoxycarbonylamino-3-methyl-butyrylamino)propanoate; and other fungicides, including guanidine, dodine, dodine free base, iminoctadine, guazatine, antibiotics: kasugamycin, oxytetracyclin and its salts, streptomycin, polyoxin, validamycin A, nitrophenyl derivatives: binapacryl, dinocap, dinobuton, sulfur-containing heterocyclyl compounds: dithianon, isoprothiolane, organometallic compounds: fentin salts, organophosphorus compounds: edifenphos, iprobenfos, fosetyl, fosetyl-aluminum, phosphorous acid and its salts, pyrazophos, tolclofos-methyl, organochlorine compounds: dichlofluanid, flusulfamide, hexachloro-benzene, phthalide, pencycuron, quintozene, thiophanate, thiophanate-methyl, tolylfluanid, others: cyflufenamid, cymoxanil, dimethirimol, ethirimol, furalaxyl, metrafenone and spiroxamine, guazatine-acetate, iminoc-tadine-triacetate, iminoctadine-tris(albesilate), kasugamycin hydrochloride hydrate, dichlorophen, pentachlorophenol and its salts, N-(4-chloro-2-nitro-phenyl)-N-ethyl-4-methyl-benzenesulfonamide, dicloran, nitrothal-isopropyl, tecnazen, biphenyl, bronopol, diphenylamine, mildiomycin, oxincopper, prohexadione calcium, N-(cyclopropylmethoxyimino-(6-difluoromethoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluormethyl-4-(3-trimethylsilanyl-propoxy)-phenyl)-N-ethyl-N-methylformamidine and N′-(5-difluormethyl-2-methyl-4-(3-trimethylsilanyl-propoxy)-phenyl)-N-ethyl-N-methyl formamidine.

25. The product of claim 23, wherein the fungicide is one or a combination of fluopyram, tebuconazole, chlorothalonil, thiophanate-methyl, prothioconazole, fludioxonil, metalaxyl, or copper hydroxide.

26. The product of claim 23, wherein the insecticide is one or a combination of pyrethroids, bifenthrin, tefluthrin, zeta-cypermethrin, organophosphates, chlorethoxyphos, chlorpyrifos, tebupirimphos, cyfluthrin, fiproles, fipronil, nicotinoids, or clothianidin.

27. The product of claim 23, wherein the insecticide comprises bifenthrin.

28. The product of claim 23, wherein the first composition further comprises one or a combination of a carrier, a surfactant, a dispersant, or a yeast extract.

29. The product of claim 23, wherein the first composition is in the form of a liquid and the Bacillus amyloliquefaciens RTI472 is present at a concentration of from about 1.0×10⁸ CFU/ml to about 1.0×10¹² CFU/ml.

30. The product of claim 23, wherein the first composition is in the form of a dust, a dry wettable powder, a spreadable granule, or a dry wettable granule and the Bacillus amyloliquefaciens RTI472 is present in an amount of from about 1.0×10⁸ CFU/g to about 1.0×10¹² CFU/g.


SEQUENCE LISTING (2 sequences)

SEQ ID NO: 1 - DNA, 1554 bp, Bacillus amyloliquefaciens
ctttatcggagagtttgatcctggctcaggacgaacgctggcggcgtgcctaatacatgcaagtcgagcggacagatgggggcttgctccctgatgtcagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctcctttct

SEQ ID NO: 2 - DNA, 3246 bp, Bacillus amyloliquefaciens
atgggtgatttccctattatgacagataccggtacttttatcatcaacggtgcagaacgtgttatcgtatctcagcttgttcggtctccaagtgtatatttcagtggtaaagtagacaaaaacggtaaaaaaggttttaccgcgactgtcattccaaaccgtggcgcatggttagaatacgaaactgatgcgaaagatgttgtgtatgtccgcattgatcgcacacgtaagttgccggttacggttcttttgcgtgctctcggcttcggttccgaccaagagattctcgatctcattggtgagaacgaatatctccgcaatacactggataaggacaacactgaaaacagtgacaaagcgcttcttgaaatctatgagcgccttcgtcccggagagccgcctacagtagaaaacgcaaaaagcttgctggattcccgtttcttcgatccgaagcgatacgaccttgcgaatgtaggacgctataaaattaataaaaagcttcatatcaagaaccgcctgtttaaccagcgccttgcagaaacactggtggatccggaaaccggtgaaattctcgctgaaaaagggcagattcttgacagaagaacacttgataaagtactgccatacttagaaaatggaatcggcttcagaaagctttatcctaacggcggcgtcgtcgaggatgaagtgatgcttcaatccattaaaatctatgctcctaccgatgcagaaggagagcagacgatcaatgtgatcggcaatgcttacatcgaagaggcgattaaaaacattacgcctgctgatattatttcttctatcagctacttcttcaacctcctgcacggagtgggcgacactgatgatatcgaccatctcggaaaccgccgtctgcgttctgtaggtgagctcctgcaaaaccaattccgtatcggtttaagccggatggaacgtgtcgtacgtgaaagaatgtctattcaagacacgaatacaattacgccgcagcagctgattaacatcagacctgttattgcgtctattaaagagttcttcggaagctcacagctttctcaattcatggatcagacgaacccgcttgctgaattgacgcacaaacgccgtctgtcagctctcggaccgggcggtttgacacgtgagcgtgcaggtatggaagtacgtgacgttcactactctcactatggccgtatgtgtccgattgaaacgcctgagggcccgaacatcggtttgatcaactcattgtcatcatttgcgaaagtaaaccgctttggtttcattgagacgccataccgccgcgttgatcctgaaacaggaaaagtaacgcctagaatcgactacctgactgctgatgaagaggataactatgtcgtagcccaagcgaatgctaagctgagcgatgacggttctttcttggatgacagcatcgtagcgcgtttcagaggggaaaacaccgttgtagcccgcaaccgcgtggattacatggacgtatctcctaaacaggttgtatctgctgcgacagcatgtattccgttcttggaaaacgatgactcgaaccgcgccctcatgggagcgaacatgcagcgtcaggctgtgcctttgatgcagccggaagctccgatcgtcggaacgggtatggaatacgtatccggtaaagactctggtgcagccgttatttgtaaacaccctggtatcgttgaacgggtggaagcgaaaaacgtatgggtgcgccgctatgaagaaattgacggccaaaaagtaaaaggcaacctggataagtacagcttgctgaaattcgtccgctccaaccaaggtacgtgctacaaccagcgtccgatcgtcagtgtcggcgatgaagtagtcaaaggagaaatccttgctgacggaccttcaatggagcttggtgaacttgctctcggccgcaacgtaatggtcggcttcatgacatgggatggttacaactatgaggatgccatcatcatgagtgaacgccttgtgaaagatgatgtatacacatctattcacattgaagaatatgaatcagaagcacgtgatacaaagcttgggccggaagagatcacccgcgatattccaaacgtaggggaagacgcgcttcgcaatcttgatgaccgcggaattatccgtatcggtgcggaagtcaacgacggagaccttctcgtaggtaaagtaacgcctaaaggtgtaactgagcttacggctgaagaacgccttcttcatgcgatctttggagaaaaagcgcgtgaagtccgtgatacttctctccgtgtgcctcacggcggcggcggaattatccacgacgtaaaagtcttcaaccgtgaagacggcgacgaacttcctccgggagtgaaccagcttgtacgcgtatatatcgttcagaaacgtaagatttctgaaggtgataaaatggccggacgtcacggaaacaaaggggttatctcgaagattcttcctgaagaagatatgccttaccttcctgacggcacgccgatcgatatcatgcttaacccgctgggtgtaccatcacgtatgaatatcggtcaggtattagaacttcacatgggtatggctgcccgctacctcggcattcacatcgcgtcacctgtatttgacggcgcgcgtgaagaagatgtgtgggaaacacttgaagaagcaggcatgtcaagagacgctaaaacagttctttatgacggccgtacgggagaaccgtttgacaaccgtgtatctgtcggaatcatgtacatgatcaaactggcgcacatggttgatgataaacttcatgcccgttctacaggtccttactcacttgttacgcagcagcctctcggcggtaaagcccaattcggcggacagcgtttcggtgagatggaggtttgggcgcttgaagcttacggcgcagcttacacgcttcaagaaatcctgactgtgaagtccgatgacgtggtcggacgtgtgaaaacatatgaagccatcgtcaaaggcgacaatgttccagagcctggtgttccggaatcattcaaagtattgatcaaagagcttcaaagcttaggtatggacgtaaaaatcctttcaggcgatgaagaagaaatagaaatgagagatctagaagacgaggaagatgcgaaacaagctgacggccttgcattatcaggtgatgaagcgccggaagaaacagcatctccagacgttgaacgtgacgcagtaacgaaagaatag